(19) United States
(12) Patent Application Publication
(10) Pub. No.: US 2026/0226029 A1
(43) Pub. Date: Aug. 6, 2026

(54) 1-(1-Oxo-1,2-Dihydrophthalazin-6-yl)Cycloalkyl-1-Carboxamide Derivatives as MTA-Cooperative Inhibitors of PRMT5

(71) Applicant: BeOne Medicines I GmbH, Basel (CH)

(72) Inventors: Sanjia Xu, Beijing (CN); Jing Li, Beijing (CN); Zhiwei Wang, Beijing (CN)

(73) Assignee: BeOne Medicines I GmbH, Basel (CH)

(21) Appl. No.: 19/576,111

(22) Filed: Mar. 24, 2026

Related U.S. Application Data
(63) Continuation of application No. PCT/IB2024/059487, filed on Sep. 27, 2024.

(30) Foreign Application Priority Data
Sep. 28, 2023 (WO) PCT/CN2023/122617

(51) Int. Cl. C07D 401/14; A61K 31/502; A61K 31/506; A61P 35/00; C07D 401/12; C07D 417/14; C07D 491/048

(52) U.S. Cl. CPC C07D 401/14; A61K 31/502; A61K 31/506; A61P 35/00; C07D 401/12; C07D 417/14; C07D 491/048

(57) ABSTRACT

This disclosure provides compounds containing 1-(1-oxo-1,2-dihydrophthalazin-6-yl)cycloalkyl-1-carboxamide derivatives, the use thereof for selectively inhibiting the activity of PRMT5 in cooperative with MTA in tumors bearing MTAP^{DEL} mutation, and pharmaceutical compositions comprising the compounds as treatment of various diseases including cancer.

### CROSS REFERENCE TO RELATED APPLICATIONS

This application is a continuation of International Application No. PCT/IB2024/059487, filed Sep. 27, 2024, which claims priority to International Application No. PCT/CN2023/122617, filed Sep. 28, 2023, the disclosures of each of which are hereby incorporated by reference in their entireties.

### FIELD OF THE INVENTION

This disclosure provides compounds containing 1-(1-oxo-1,2-dihydrophthalazin-6-yl)cycloalkyl-1-carboxamide derivatives, the use thereof for selectively inhibiting the activity of PRMT5 in cooperative with MTA in tumors bearing MTAP^{DEL} mutation, and pharmaceutical compositions comprising the compounds as treatment of various diseases including cancer.

### BACKGROUND OF THE INVENTION

Epigenetic modification is a process that can modify genetic output changing the primary DNA sequence. Epigenetic modification plays an important role in gene expression and regulation, protein production and cell differentiation in multiple dimensions. Typically, this process is reversible and selective, on DNA, its regulatory proteins such as histones and other proteins such as transcription factors [Bradbury, E. M., BioEssays, 1992, 14 (1): pp. 9-16]. PMTs (Protein Methyltransferases) are central players on epigenetic modifications, consisting of two sub-families named PKMTs (Protein Lysine Methyltransferases) and PRMTs (Protein Arginine Methyltransferases) [Copeland, R. A., et al., Oncogene, 2012. 32 (8): pp. 939-46]. PMTs are associated with various human diseases and considered as potential therapeutic targets [Copeland, R. A., et al., Oncogene, 2012, 32 (8): pp. 939-46].

As the name implies, PRMTs catalyze the methylation of the arginine residues of proteins. Besides their primary functions of methylating the histone tails, PRMTs also target on other cellular proteins such as NAB2p, FOXO1, PABP1, Sm D1, etc. [Bedford, M. T., et al., Molecular Cell, 2005, 18 (3): pp. 263-72]. Divided by the products, the 9 mammalian PRMTs can be classified into 3 subtypes: type I (PRMT1, PRMT2, PRMT3, PRMT4, PRMT6 and PRMT8) catalyzes aDMA (asymmetrical dimethylated arginine) formation; type II (PRMT5, PRMT9) catalyzes sDMA (symmetrical dimethylated arginine); and type III (PRMT7) catalyzes MMA (monomethylated arginine) formation [Yang, Y., et al., Nature Reviews Cancer, 2012, 13 (1): pp. 37-50]. In addition, type I/II PRMTs can also catalyze MMA formation as an intermediate to aDMA and sDMA. The PRMTs comprise a pocket to interact with its cofactor SAM (S-adenosyl methionine), and an adjacent pocket to interact with the arginine residue on a protein, namely SAM-pocket and substrate-pocket. The methylation process involves an S_{N}2-like mechanism of transferring an activated methyl group from cofactor SAM to the guanidino group on the arginine residue. [Bedford, M. T., et al., Molecular Cell, 2005, 18 (3): pp. 263-72]. The side product of the process is SAH (S-adenosyl-L-homocysteine).

The overall arginine level in cells is roughly 1500:3:2:1 for Arg:aDMA:MMA:sDMA, and PRMT5 accounts for the vast majority of sDMA formation [Dhar, S., et al., Scientific Reports, 2013, 3: 1311]. In contrast with PRMT1, the major type I PRMT which functions on its own in cells, PRMT5 binds to MEP50 (Methylosome Protein 50) to form a heterocomplex that is often elevated in cancer cells and correlates to poor patient survival [Gao, G., et al., Nucleic Acids Research, 2019, 47 (10): pp. 5038-48]. PRMT5 promotes tumerigenesis in varied mechanisms. PRMT5 is a strong repressor of numerous genes; when PRMT5 methylates histones H2a and H4 on Arg3 and histone H3 on Arg8, it represses gene transcripts that involved in differentiation, transformation, cell-cycle progression and tumor suppression [Karkhanis, V., et al., Trends in Biochemical Sciences, 2011, 36 (12): pp. 633-41]. Besides its epigenetic roles, PRMT5 may also regulates RNA-binding proteins such as splicing factors. For instance, a reproducible event was observed in PRMT5 knockout mice, in which exon 6 skipping of MDM4 (Murine Double Minute 4) occurred and p53 was released to upregulate p53 pathway [Gerhart, S. V, et al., Scientific Reports, 2018, 8: 9711]. In addition, PRMT5 could directly influence key proliferation pathways by direct methylation of p53 [Jansson, M, et al., Nature Cell Biology, 2008, 10 (12): pp. 1431-9], EGFR [Hsu, J.-M., et al., Nature Cell Biology, 2011, 13 (2): pp. 174-81], PI3K [Wei, T.-Y. W., et al., Cellular Signaling, 2014, 26 (12): pp. 2940-50], etc. Thus, PRMT5 has a good potential to become a clinically relevant target.

On the other hand, PRMT5 is an essential gene in normal tissues, and the systemic inhibition of PRMT5 may result in significant liabilities, especially hematologic toxicity [Ahnert, J. R., et al., Journal of Clinical Oncology, 2021, 39 (15-suppl): p. 3019]. Therefore, strategies to selectively block the PRMT5 activities in tumor cells are required for a safer therapy.

Homozygous deletion of tumor depressor CDKN2A (Cyclin Dependent Kinase Inhibitor 2A) occurs in about 15% of all tumor types. Interestingly, the mutation frequently involves the co-deletion of proximate genes existing in 9p21, including the gene that encodes MTAP (Methylthioadenosine Phosphorylase) [Firestone, R. S., et al., Journal of American Chemical Society, 2017, 139 (39): p. 13754-60]. As a result of MTAP deletion, MTA (methylthioadenosine), the substrate of MTAP, accumulates. MTA is structurally related to SAM, and is a weak ligand/inhibitor of PRMT5 that occupies the same pocket with SAM. The formation of MTA-PRMT5 complex provides chances for further PRMT5 inhibition by formation of a tertiary complex. In such way, a correlation of MTAP null status and dependency of PRMT5 is established through MTA concentration level, to provide a precise oncological therapy.

Currently, most of the clinical-stage PRMT5 inhibitors are unable to differentiate normal cells and cancer cells, based on a SAM/MTA competitive mechanism (JNJ64619178, PF06939999, PRT543, and PRT811) or a non-MTA cooperative mechanism (GSK3326595). There thus remain unmet and continuous medical needs for potent and selective MTA-cooperative PRMT5 inhibitors.

### SUMMARY OF THE INVENTION

One objective of the present invention is to provide compounds and derivatives which function to act as PRMT5 inhibitors, and methods of preparation and uses thereof.

Aspect 1. A compound of Formula (I):

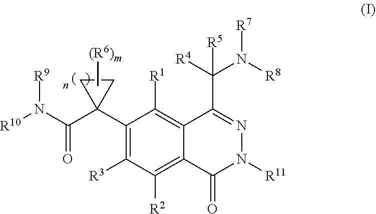

or a N-oxide thereof, or a pharmaceutically acceptable salt thereof, or a stereoisomer thereof, or a tautomer thereof, or a deuterated analog thereof, wherein:
n is 1, 2, 3 or 4;
m is 1, 2, 3, or 4;
R¹ and R² are each independently selected from hydrogen, halogen, —C₁₋₈alkyl, —C₃-C₈cycloalkyl, —CN, —OR^{1a}, —NR^{1a}R^{1b}, —COR^{1a}, —CO₂R^{1a}, —CONR^{1a}R^{1b} or —NR^{1a}COR^{1b}, wherein each of —C₁₋₈alkyl and —C₃-C₈cycloalkyl is optionally substituted with at least one substituent selected from halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R³, R⁴, R⁵ and R⁶ are each independently selected from hydrogen, halogen, —C₁₋₈alkyl, —C₃-C₈cycloalkyl, —CN, —OR^{3a}, —NR^{3a}R^{3b}, —COR^{3a}, —CO₂R^{3a}, —CONR^{3a}R^{3b} or —NR^{3a}COR^{3b}, wherein each of —C₁₋₈alkyl and —C₃-C₈cycloalkyl is optionally substituted with at least one substituent selected from halogen, —C₁₋₈alkoxy, —C₁₋₈alkyl, —C₃-C₈cycloalkyl, 3- to 8-membered heterocyclyl, C₆-C₁₂aryl, 5- to 12-membered heteroaryl, oxo, —CN, —OR^{3c}, —SO₂R^{3c}, —SO₂NR^{3c}R^{3d}, —COR^{3c}, —CO₂R^{3c}, —CONR^{3c}R^{3d}, —NR^{3c}R^{3d}, —NR^{3c}COR^{3d}, —NR^{3c}CO₂R^{3d}, or —NR^{3c}SO₂R^{3d};
R^{3a}, R^{3b}, R^{3c} and R^{3d} are each independently hydrogen, —C₁₋₈alkyl, C₃-C₈cycloalkyl, 3- to 8-membered heterocyclyl, —C₆-C₁₂aryl, or 5- to 12-membered heteroaryl; each of said —C₁₋₈alkyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁷ and R⁸ are each independently selected from hydrogen, —C₁₋₈alkyl, —C₃-C₈cycloalkyl or —C₆-C₁₂aryl, wherein each of —C₁₋₈alkyl, —C₃-C₈cycloalkyl and —C₆-C₁₂aryl is optionally substituted with at least one substituent selected from hydrogen, halogen, —C₁₋₈alkyl, —C₂₋₈alkenyl, —C₂₋₈alkynyl, —NR^{7a}R^{7b}, —OR^{7a}, oxo, —C₃-C₈cycloalkyl, 3- to 8-membered heterocyclyl, —C₆-C₁₂aryl, 5- to 12-membered heteroaryl, or —CN;
R^{7a} and R^{7b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, wherein each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{7C};
R^{7c} is independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl or —CN, wherein each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl is optionally substituted with at least one hydrogen, halogen, hydroxy, —C₁₋₈alkyl, —C₁₋₈alkoxy, —CN, —NH₂ or oxo;
R⁹ and R¹⁰ are each independently selected from hydrogen, —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{9a}; or
R⁹ and R¹⁰ together with the nitrogen atom to which they are attached, form a 3- to 8-membered unsaturated or saturated ring, said ring comprising 0-3 additional heteroatoms independently selected from nitrogen, oxygen or sulfur and said ring is optionally substituted with at least one substituent R^{9a};
R^{9a} is independently hydrogen, halogen, deuterium, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9b}, —SO₂R^{9b}, —SO₂NR^{9b}R⁹⁰, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{9d}; or
two R^{9a} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent R^{9d};
R^{9b} and R^{9c} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{9e};
R^{9d} and R^{9e} are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9′}, —SO₂R^{9f}, —SO₂NR^{9f}R^{9g}, —COR^{9f}, —CO₂R^{9f}, —CONR^{9f}R^{9g}, —NR^{9f}R^{9g}, —NR^{9f}COR^{9g}, —NR^{9f}CO₂R^{9g}, —NR^{9f}SO₂R^{9g}, oxo, —SF₅ or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{9h};
R^{9f} and R^{9g} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R⁹ⁱ;
R^{9h} and R⁹ⁱ are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9j}, —SO₂R^{9j}, —SO₂NR^{9j}R^{9k}, —COR^{9j}, —CO₂R^{9j}, —CONR^{9j}R^{9k}, —NR^{9j}R^{9k}, —NR^{9j}COR^{9k}, —NR^{9j}CO₂R^{9k}, —NR^{9j}SO₂R^{9k}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo;
R^{9j} and R^{9k} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo.

Aspect 2. The compound of Aspect 1, wherein the compound is formula (IIa) or (IIb):

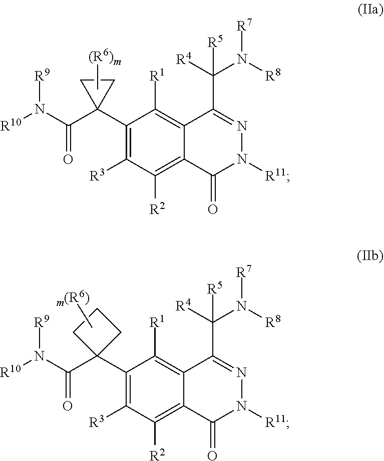

preferably, the compound is selected from formula (IIc) or (IId):

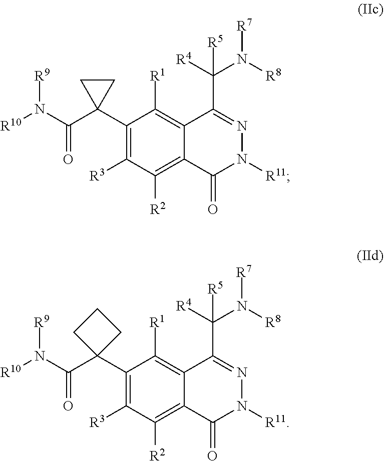

Aspect 3. The compound of Aspect 1, wherein the compound is formula (IIIa):

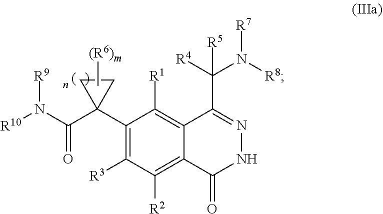

preferably, the compound is selected from formula (IIIb):

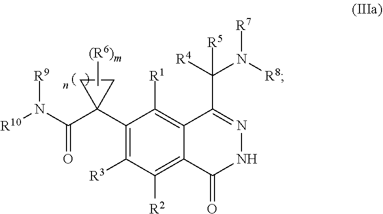

more preferably, the compound is selected from formula (IIIc):

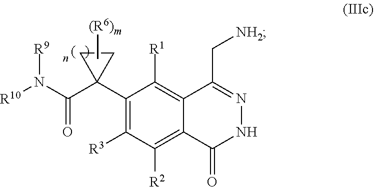

more preferably, the compound is selected from formula (IIId):

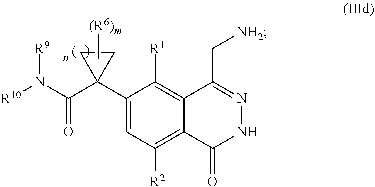

Aspect 4. The compound of Aspect 1, wherein the compound is formula (IVa) or (IVb):

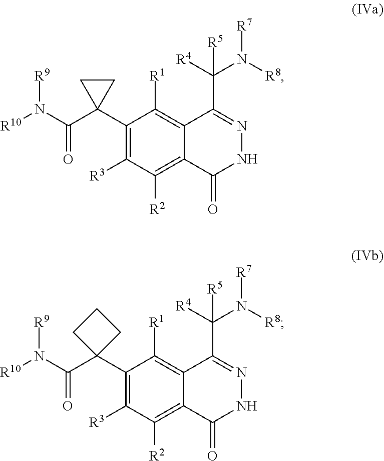

preferably, the compound is selected from formula (IVc) or (IVd):

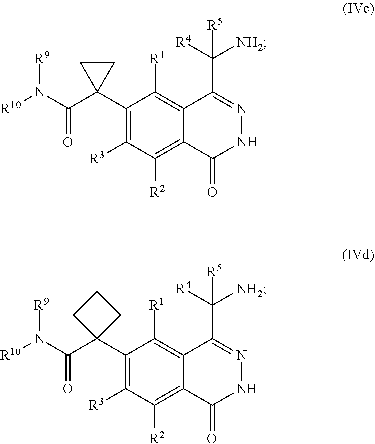

more preferably, the compound is selected from formula (IVe) or (IVf):

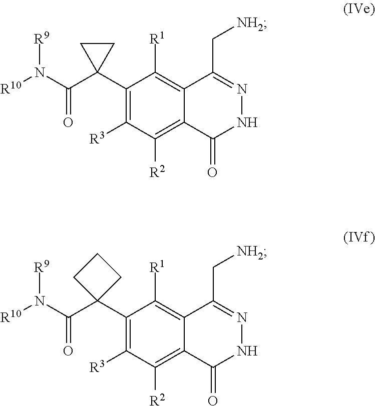

even more preferably, the compound is selected from formula (IVg) or (IVh):

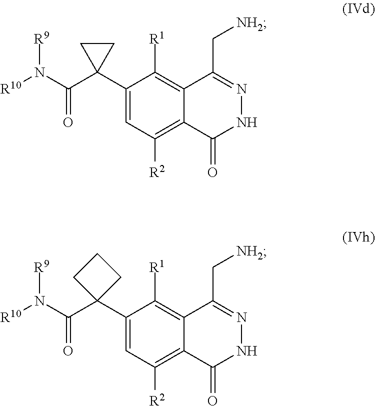

even more preferably, the compound is selected from formula (IVi) or (IVj):

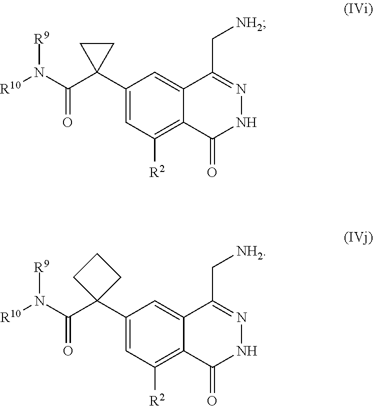

Aspect 5. The compound of Aspect 1, wherein the compound is formula (Va):

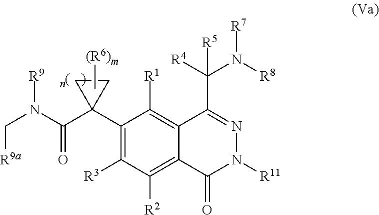

preferably, the compound is selected from formula (Vb) or (Vc):

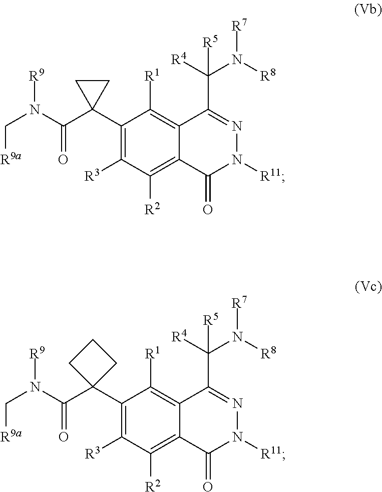

more preferably, the compound is selected from formula (Vd) or (Ve):

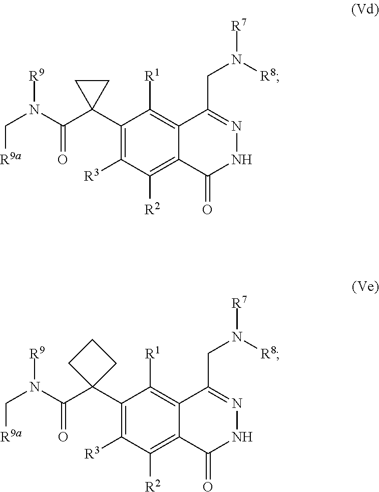

even more preferably, the compound is selected from formula (Vf) or (Vg):

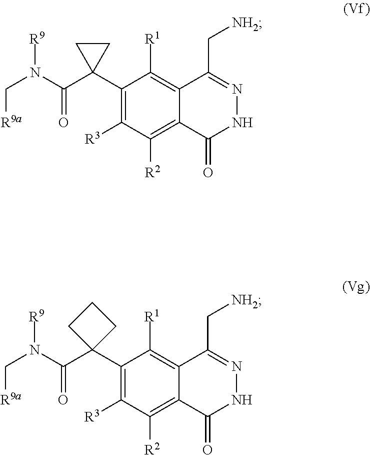

even more preferably, the compound is selected from formula (Vh) or (Vi):

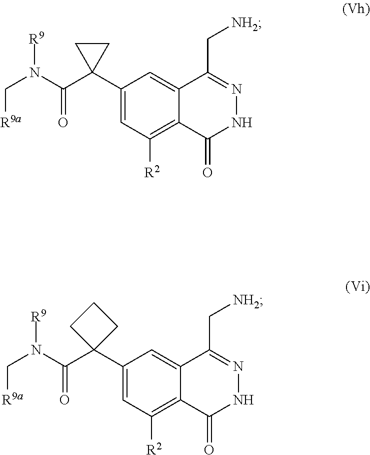

wherein, R^{9a} is as defined as aspect 1.

Aspect 6. The compound of anyone of the preceding Aspects, wherein R¹ and R² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{1a}, —NR^{1a}R^{1b}, —COR^{1a}, —CO₂R^{1a}, —CONR^{1a}R^{1b} or —NR^{1a}COR^{1b}, wherein each of methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent selected from —F, —Cl, —Br, —I, methoxy, ethoxy, propoxy, butoxy, pentoxy, hexoxy, heptoxy, octoxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 7. The compound of anyone of the preceding Aspects, wherein R¹ and R² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{1a}, —NR^{1a}R^{1b}, —COR^{1a}, —CO₂R^{1a}, —CONR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.
preferably, R¹ and R² are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN;
more preferably, R¹ and R² are each independently selected from hydrogen, —F, —Cl, methyl, ethyl, propyl (iso-propyl or n-propyl), butyl (n-butyl, sec-butyl, iso-butyl or tert-butyl), cyclopropyl, cyclobutyl.
even more preferably, R¹ and R² are each independently selected from hydrogen or methyl.

Aspect 8. The compound of anyone of the preceding Aspects, wherein R³, R⁴, R⁵ and R⁶ are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OR^{3a}, —NR^{3a}R^{3b}, —COR^{3a}, —CO₂R^{3a}, —CONR^{3a}R^{3b} or —NR^{3a}COR^{3b}, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl or cyclooctyl is optionally substituted with at least one substituent selected from —F, —Cl, —Br, —I, methoxy, ethoxy, propoxy, butoxy, pentoxy, hexoxy, heptoxy, octoxy, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{3c}, —SO₂R^{3c}, —SO₂NR^{3c}R^{3d}, —COR^{3c}, —CO₂R^{3c}, —CONR^{3c}R^{3d}, —NR^{3c}R^{3d}, —NR^{3c}COR^{3d}, —NR^{3c}CO₂R^{3d}, or —NR^{3c}SO₂R^{3d};
R^{3a}, R^{3b}, R^{3c} and R^{3d} are each independently hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R³, R⁴, R⁵ and R⁶ are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OR^{3a}, —NR^{3a}R^{3b}, —COR^{3a}, —CO₂R^{3a}, —CONR^{3a}R^{3b} or —NR^{3a}COR^{3b};
R^{3a} and R^{3b} are each independently hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
more preferably, R³, R⁴, R and R are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OH, methoxy, ethoxy, propoxy, butoxy, pentoxy, hexoxy, heptoxy, octoxy;
even more preferably, R³, R⁴, R⁵ and R⁶ are each independently selected from hydrogen, —F, —Cl, methyl, ethyl, propyl (iso-propyl or n-propyl), butyl (n-butyl, sec-butyl, iso-butyl or tert-butyl);
even more preferably, R³, R⁴, R⁵ and R⁶ are each independently selected from hydrogen.

Aspect 9. The compound of anyone of the preceding Aspects, wherein R¹ and R⁸ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl is optionally substituted with at least one substituent selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, —NR^{7a}R^{7b}, —OR^{7a}, oxo, cyclopropyl, cyclobutyl, cyclopentyl, cyclohexyl, cycloheptyl, cyclooctyl 3- to 8-membered heterocyclyl, phenyl, 5- to 12-membered heteroaryl, or —CN;
R^{7a} and R^{7b} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7c};
R^{7c} is independently —F, —Cl, —Br, —I, hydroxy, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN, wherein each of said hydroxy, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one hydrogen, halogen, hydroxy, —C₁₋₈alkyl, —C₁₋₈alkoxy, —CN, —NH₂ or oxo;
preferably, R⁷ and R⁸ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl is optionally substituted with at least one substituent selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, —NR^{7a}R^{7b}, —OR^{7a}, oxo, cyclopropyl, cyclobutyl, cyclopentyl, cyclohexyl, cycloheptyl, cyclooctyl 3- to 8-membered heterocyclyl, phenyl, 5- to 12-membered heteroaryl, or —CN;
R^{7a} and R^{7b} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R⁷ and R⁸ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl;
even more preferably, R⁷ and R⁸ are each independently selected from hydrogen.

Aspect 10. The compound of anyone of the preceding aspects, wherein R⁹ and R¹⁰ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl and 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur and said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, naphthalenyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9b}, —SO₂R^{9b}, —SO₂NR^{9b}R^{9c}, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl or 5- to 12-membered heteroaryl is optionally substituted with at least one substituent R^{9d}; or
two R^{9a} together with the atom(s) to which they are attached, form a 3-, 4-, 5-, 6-, 7- or 8-membered unsaturated or saturated ring, said ring comprising 0, 1, 2 or 3 heteroatoms independently selected from nitrogen, oxygen or sulfur and said ring is optionally substituted with at least one substituent R^{9d};
R^{9b} and R^{9c} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl or 5- to 12-membered heteroaryl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9f}, —SO₂R^{9f}, —SO₂NR^{9f}R^{9g}, —COR^{9f}, —CO₂R^{9f}, —CONR^{9f}R^{9g}, —NR^{9f}R^{9g}, —NR^{9f}COR^{9g}, —NR^{9f}CO₂R^{9g}, —NR^{9f}SO₂R^{9g}, oxo, —SF₅ or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, and 5- to 12-membered heteroaryl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl, naphthalenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl, naphthalenyl or 5- to 12-membered heteroaryl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9j}, —SO₂R^{9j}, —SO₂NR^{9j}R^{9k}, —COR^{9j}, —CO₂R^{9j}, —CONR^{9j}R^{9k}, —NR^{9j}R^{9k}, —NR^{9j}COR^{9k}, —NR^{9j}CO₂R^{9k}, —NR^{9j}SO₂R^{9k}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl and 5- to 12-membered heteroaryl is optionally substituted with at least one substituent —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OH, —NH₂ or oxo;
R^{9j} and R^{9k} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl,
wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl is optionally substituted with at least one substituent —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OH, —NH₂ or oxo.

Aspect 11. The compound of anyone of the preceding aspects, wherein R⁹ and R¹⁰ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, tetrahydrofuropyridinyl —OR^{9b}, —SO₂R^{9b}, —SO₂NR^{9b}R^{9c}, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} and R^{9c} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —COR^{9f}, —CO₂R^{9f}, —CONR^{9f}R^{9g}, —NR^{9f}R^{9g}, —NR^{9f}COR^{9g}, —NR^{9f}CO₂R^{9g}, oxo, —SF₅, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9j}, —COR^{9j}, —CO₂R^{9j}, —CONR^{9j}R^{9k}, —NR^{9j}R^{9k}, —NR^{9j}COR^{9k}, —NR^{9j}CO₂R^{9k}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —CN, —OH, —NH₂ or oxo;
R^{9j} and R^{9k} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —CN, —OH, —NH₂ or oxo;
preferably, R⁹ and R¹⁰ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl (preferably, 2,3-dihydrofuro[2,3-b]pyridinyl), tetrahydrofuropyridinyl, —OR^{9b} or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} is each independently hydrogen, methyl, ethyl, propyl (iso-propyl or n-propyl), cyclopropyl or cyclobutyl;
R^{9d} is each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —NR^{9f}R⁹, —NR^{9f}COR^{9g}, oxo, —SO₂R^{9f}, —SF₅, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OH, or —CN;
more preferably, R⁹ and R¹⁰ are each independently selected from hydrogen, methyl, ethyl, propyl (iso-propyl or n-propyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 6-membered unsaturated or saturated ring, said ring is optionally substituted with at least one substituent R^{9a};
R^{9a} is independently hydrogen, —F, —Cl, —Br, —I, methyl, ethyl, propyl (iso-propyl or n-propyl), cyclopropyl, cyclobutyl, cyclopentyl, cyclohexyl, phenyl, naphthalenyl, pyrimidinyl, pyridazinyl, pyrazinyl, dihydrofuropyridinyl (preferably, 2,3-dihydrofuro[2,3-b]pyridinyl), benzo[d]thiazolyl, pyridinyl, quinolinyl, isoquinolinyl, thiazolyl, —OR^{9b} or —CN wherein each of said methyl, ethyl, propyl (iso-propyl or n-propyl), phenyl, naphthalenyl, pyrimidinyl, pyridazinyl, pyrazinyl, dihydrofuropyridinyl (preferably, 2,3-dihydrofuro[2,3-b]pyridinyl), benzo[d]thiazolyl, pyridinyl, quinolinyl, isoquinolinyl or thiazolyl is optionally substituted with at least one substituent R^{9d};
R^{9b} is each independently hydrogen, methyl, ethyl, propyl (iso-propyl or n-propyl), cyclopropyl or cyclobutyl;
R^{9d} is each independently hydrogen, —F, —Cl, —Br, —I, methyl, ethyl, propyl (iso-propyl or n-propyl), butyl (n-butyl, sec-butyl, iso-butyl or tert-butyl), phenyl, pyrimidinyl, tetrahydro-2H-pyranyl, pyridazinyl, pyrazinyl, triazolyl (preferably, 1H-1,2,4-triazolyl, 4H-1,2,4-triazolyl), pyridinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —SF₅ or —CN, wherein each of said methyl, ethyl, propyl (iso-propyl or n-propyl), butyl (n-butyl, sec-butyl, iso-butyl or tert-butyl), phenyl, pyrimidinyl, tetrahydro-2H-pyranyl, pyridazinyl, pyrazinyl, triazolyl (preferably, 1H-1,2,4-triazolyl, 4H-1,2,4-triazolyl), pyridinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} is each independently selected from hydrogen, methyl, ethyl, propyl (iso-propyl or n-propyl), cyclopropyl, cyclobutyl, cyclopentyl, cyclohexyl or phenyl, wherein each of said methyl, ethyl, propyl (iso-propyl or n-propyl), cyclopropyl, cyclobutyl, cyclopentyl, cyclohexyl or phenyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, —OH, or —CN.

Aspect 12. The compound of anyone of the preceding Aspects, wherein R⁹ and R¹⁰ are each independently selected from —C₁₋₈alkyl, —C₃-C₈cycloalkyl, —C₃-C₁₂cycloalkenyl and 3- to 12-membered heterocyclyl, wherein each of said —C₁₋₈alkyl, —C₃-C₈cycloalkyl, —C₃-C₁₂cycloalkenyl and 3- to 12-membered heterocyclyl is optionally substituted with at least one substituent R^{9a}; or
R⁹ and R¹⁰ together with the nitrogen atom to which they are attached, form a 3- to 8-membered unsaturated or saturated ring, said ring comprising 0-3 additional heteroatoms independently selected from nitrogen, oxygen or sulfur and said ring is optionally substituted with at least one substituent R^{9a};
R^{9a} is independently hydrogen, halogen, deuterium, —C₁₋₈alkyl, —C₃-C₈cycloalkyl, —C₆-C₁₂aryl, 5- to 12-membered heteroaryl, —OR^{9b} or —CN, wherein each of said —C₁₋₈alkyl, —C₃-C₈cycloalkyl, —C₆-C₁₂aryl and 5- to 12-membered heteroaryl is optionally substituted with at least one substituent R^{9d}; or
two R^{9a} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent R^{9d};
R^{9b} is each independently selected from hydrogen, —C₁₋₈alkyl, wherein said —C₁₋₈alkyl is optionally substituted with at least one substituent R^{9e};
R^{9d} and R^{9e} are each independently hydrogen, halogen, —C₁₋₈alkyl, —C₃-C₈cycloalkyl, —C₆-C₁₂aryl, 5- to 12-membered heteroaryl, —OR^{9f}, —SO₂R^{9f}, —SF₅ or —CN, wherein each of said —C₁₋₈alkyl, —C₆-C₁₂aryl and 5- to 12-membered heteroaryl is optionally substituted with at least one substituent R^{9h};
R^{9f} is each independently selected from hydrogen, —C₁₋₈alkyl and —C₃-C₈cycloalkyl, —C₆-C₁₂aryl, wherein each of said —C₁₋₈alkyl, —C₆-C₁₂aryl, and —C₃-C₈cycloalkyl is optionally substituted with at least one substituent R⁹ⁱ;
R^{9h} and R⁹ⁱ are each independently hydrogen, halogen, —C₁₋₈alkyl or —CN;
preferably, R⁹ and R¹⁰ are each independently selected from methyl, ethyl, propyl (iso-propyl or n-propyl), butyl (n-butyl, sec-butyl, iso-butyl or tert-butyl), cyclopropyl, cyclobutyl, cyclopentyl, cyclohexyl, chromanyl, isochromanyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, chromanyl, isochromanyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, pyrimidinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl (preferably, 2,3-dihydrofuro[2,3-b]pyridinyl), tetrahydrofuropyridinyl —OR^{9b} or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, pyrimidinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} is each independently selected from hydrogen, methyl, ethyl, propyl (iso-propyl or n-propyl), butyl (n-butyl, sec-butyl, iso-butyl or tert-butyl), pentyl, hexyl, heptyl and octyl, wherein each of said methyl, ethyl, propyl (iso-propyl or n-propyl), butyl (n-butyl, sec-butyl, iso-butyl or tert-butyl), pentyl, hexyl, heptyl and octyl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —SF₅ or —CN, wherein each of said methyl, ethyl, propyl (iso-propyl or n-propyl), butyl (n-butyl, sec-butyl, iso-butyl or tert-butyl), pentyl, hexyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} is each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl or naphthalenyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl or naphthalenyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl or —CN.

Aspect 13. The compound of anyone of the preceding Aspects, wherein R⁹ and R¹⁰ are each independently selected from -Me, -Et, —Pr (-nPr or -isoPr), -Bu

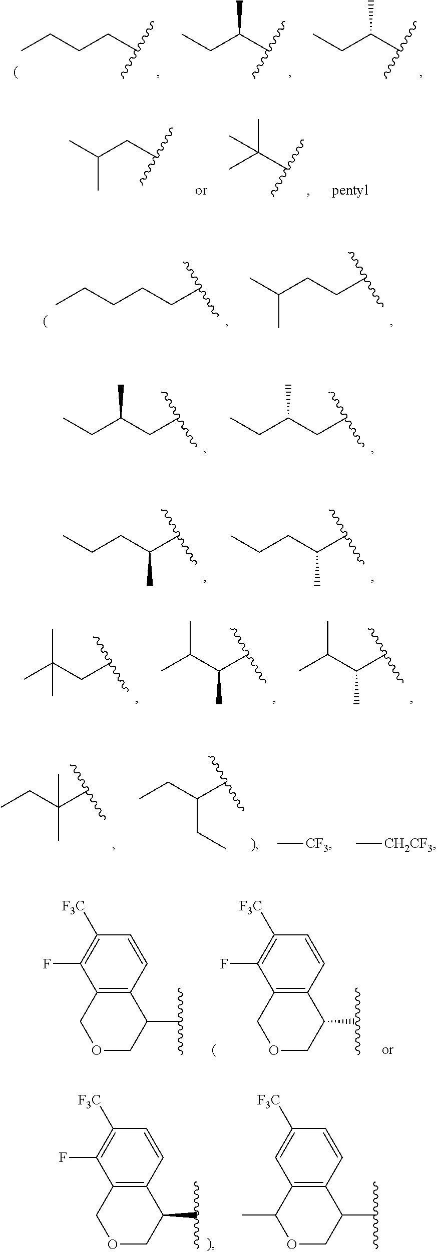

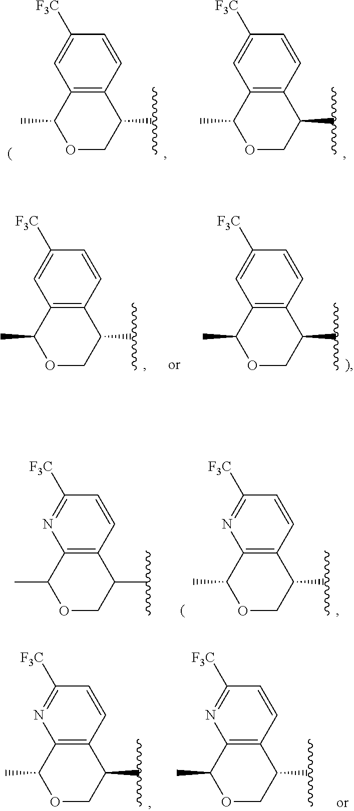

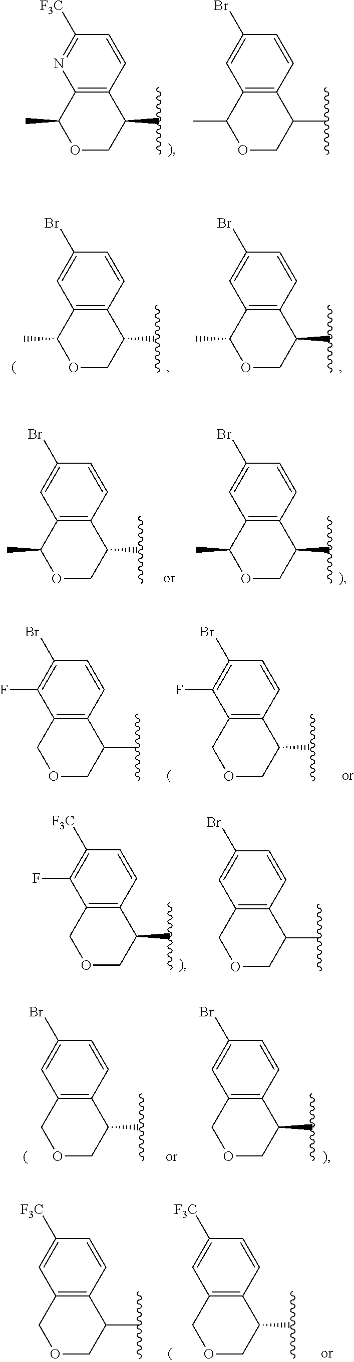

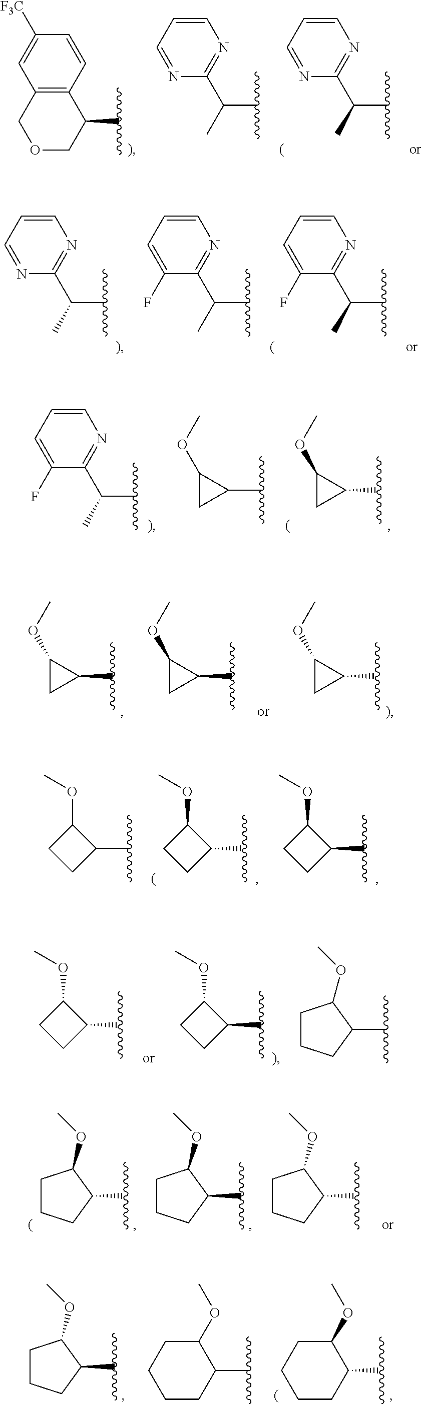

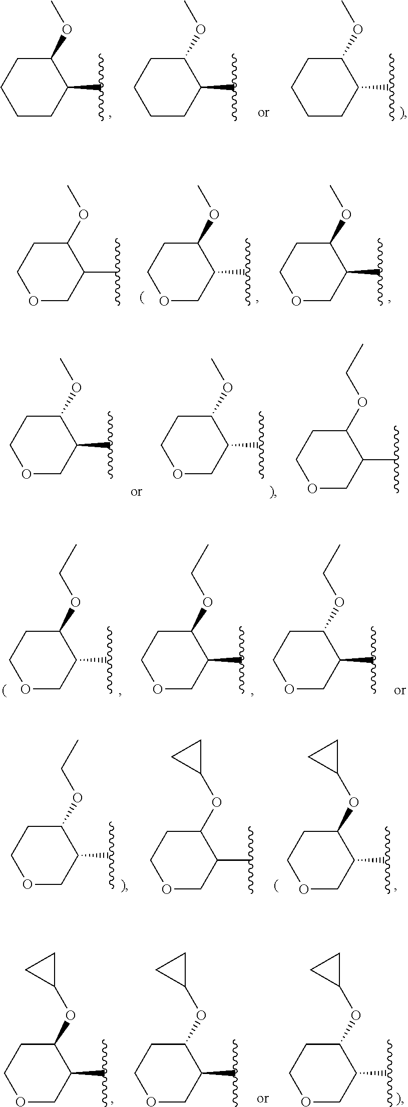

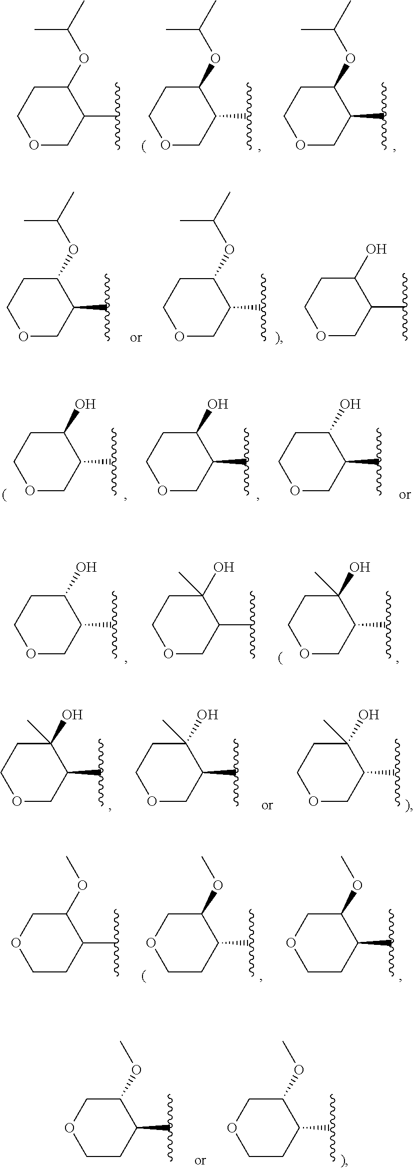

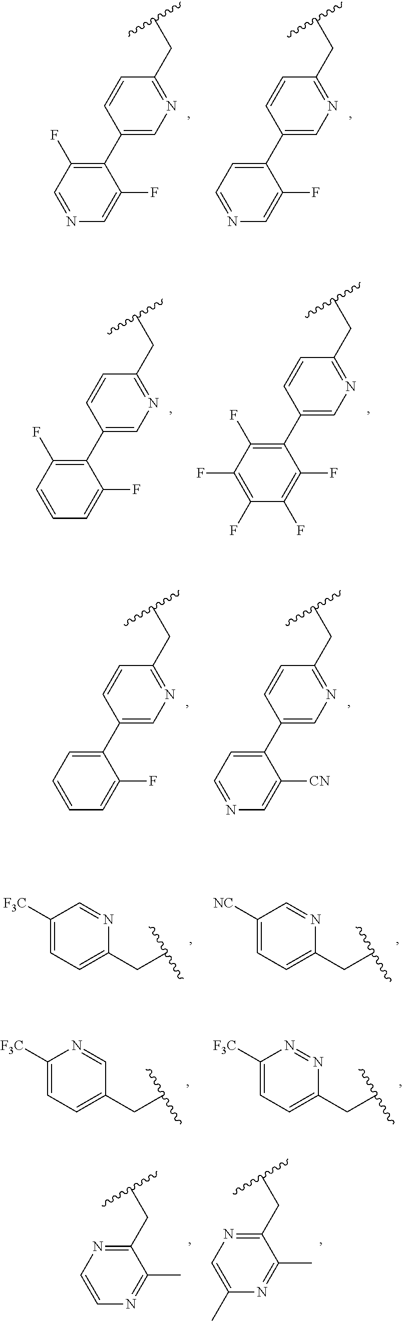

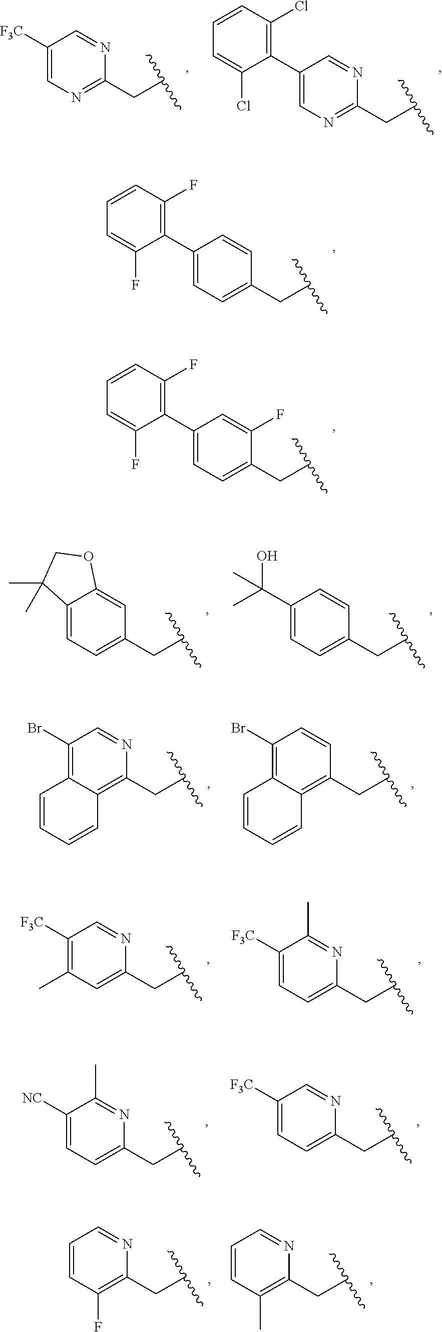

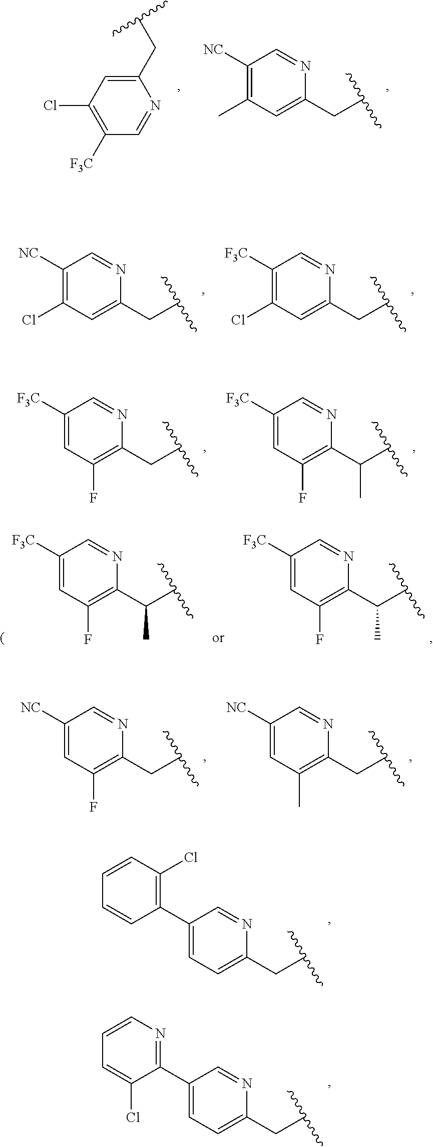

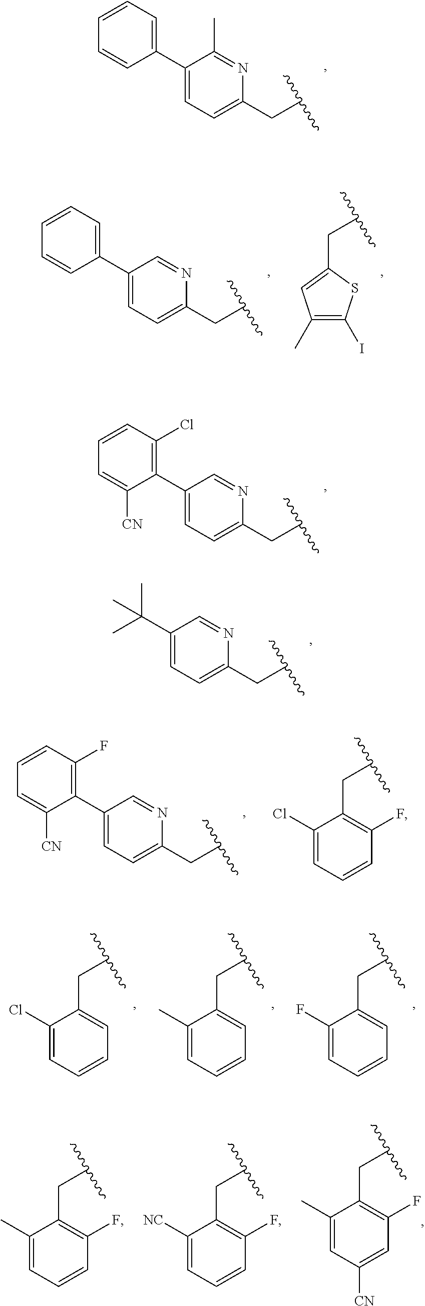

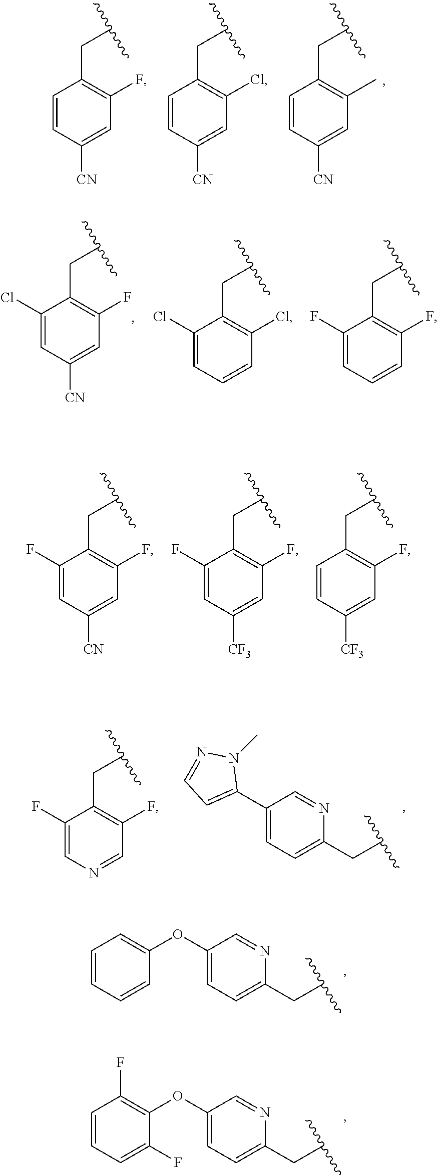

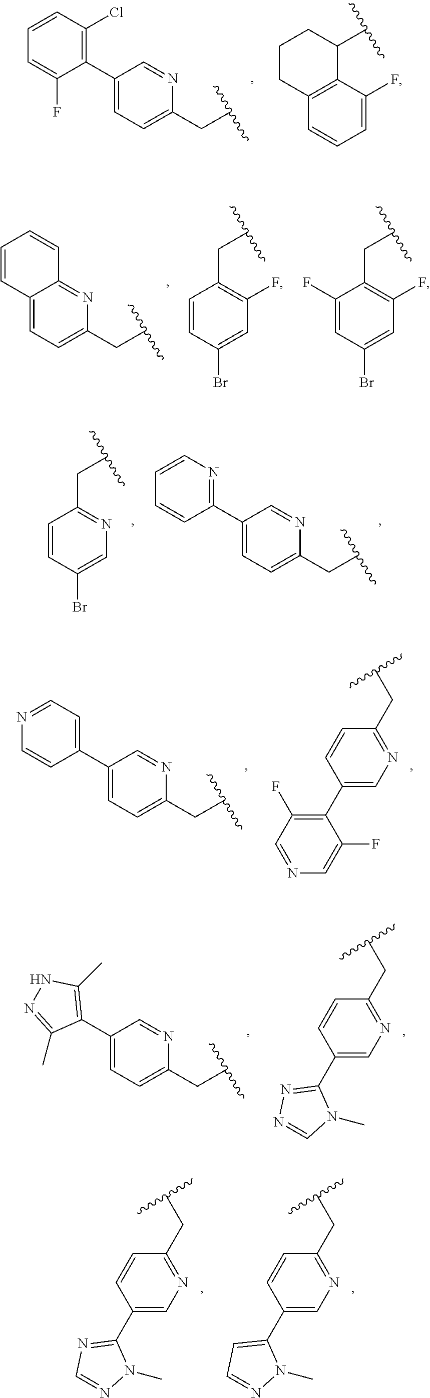

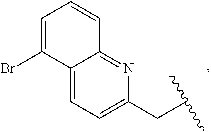 [SMILES: *Cc1ccc2c(Br)cccc2n1]

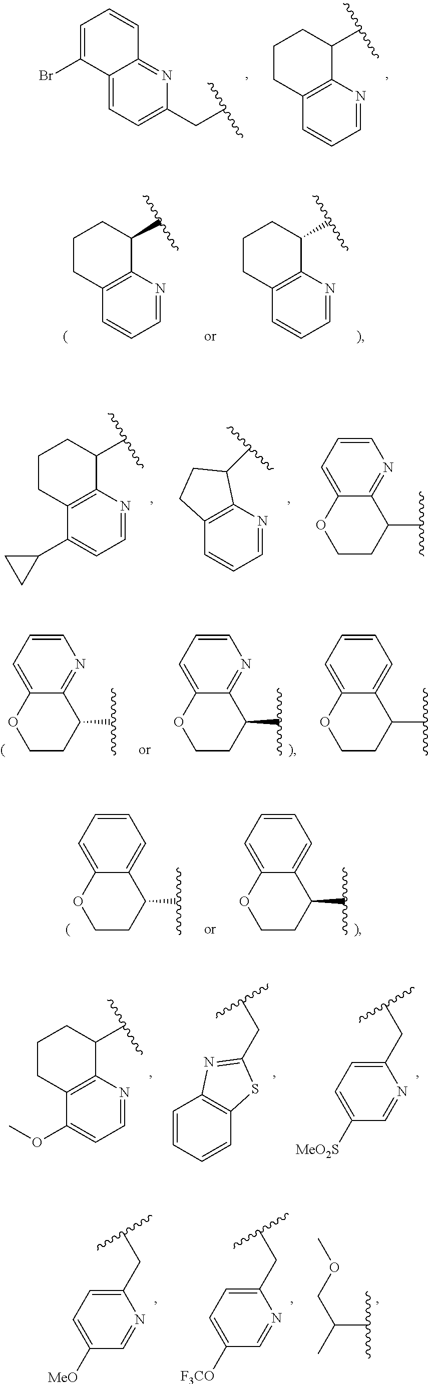

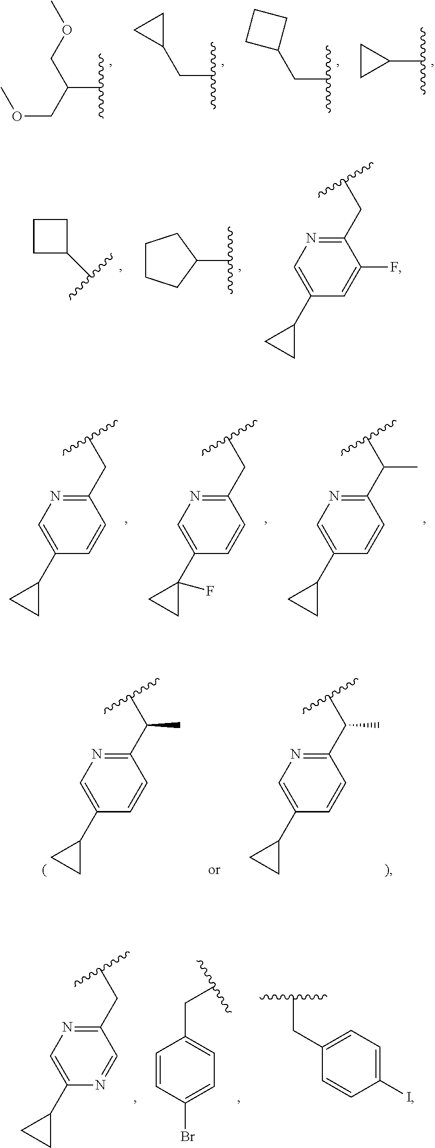

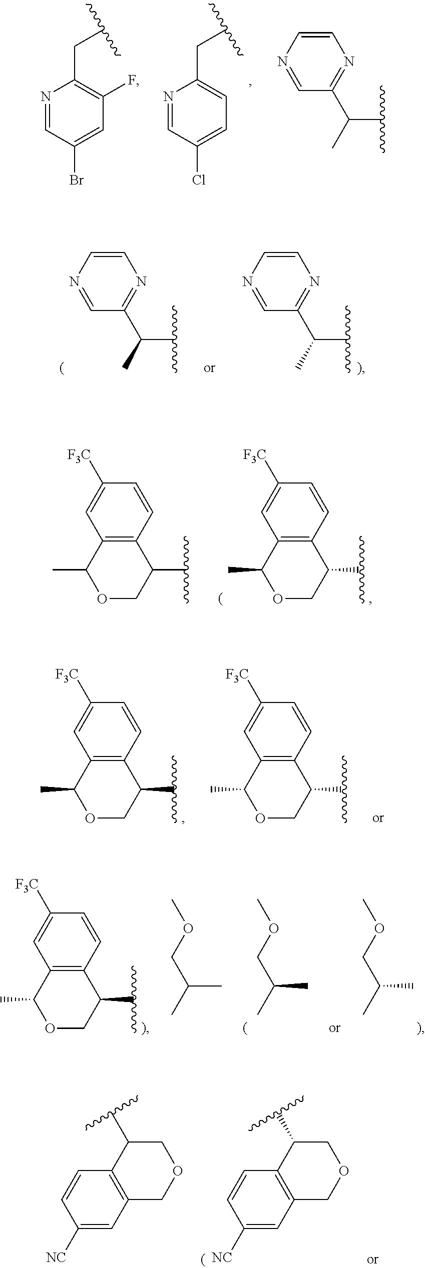

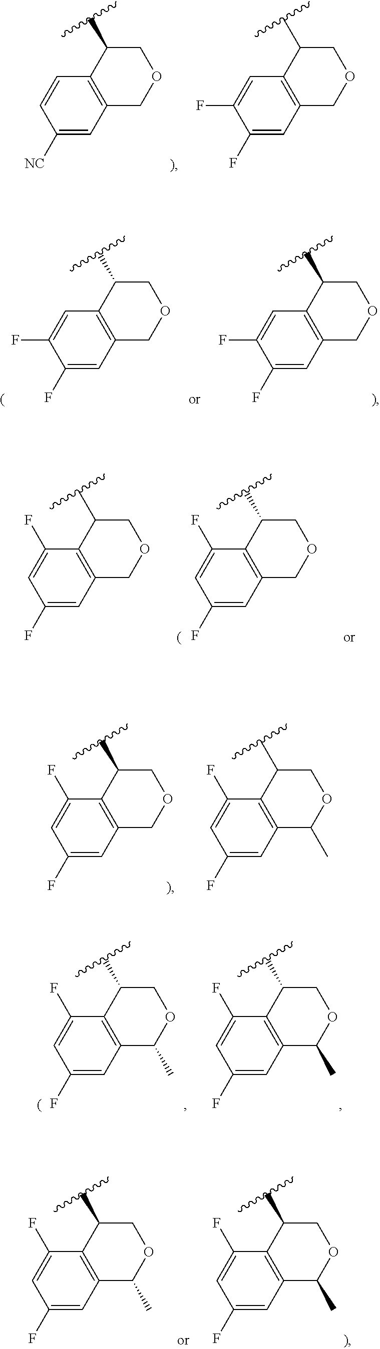

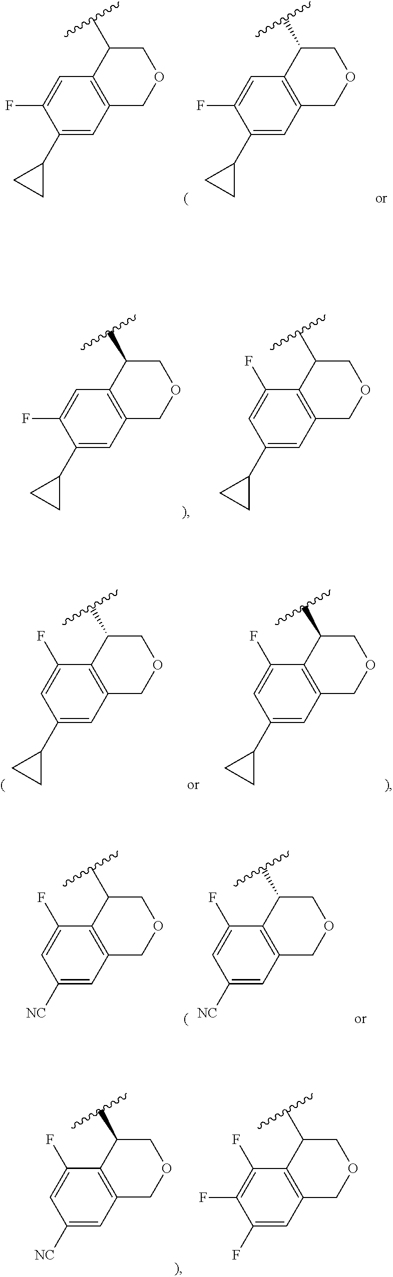

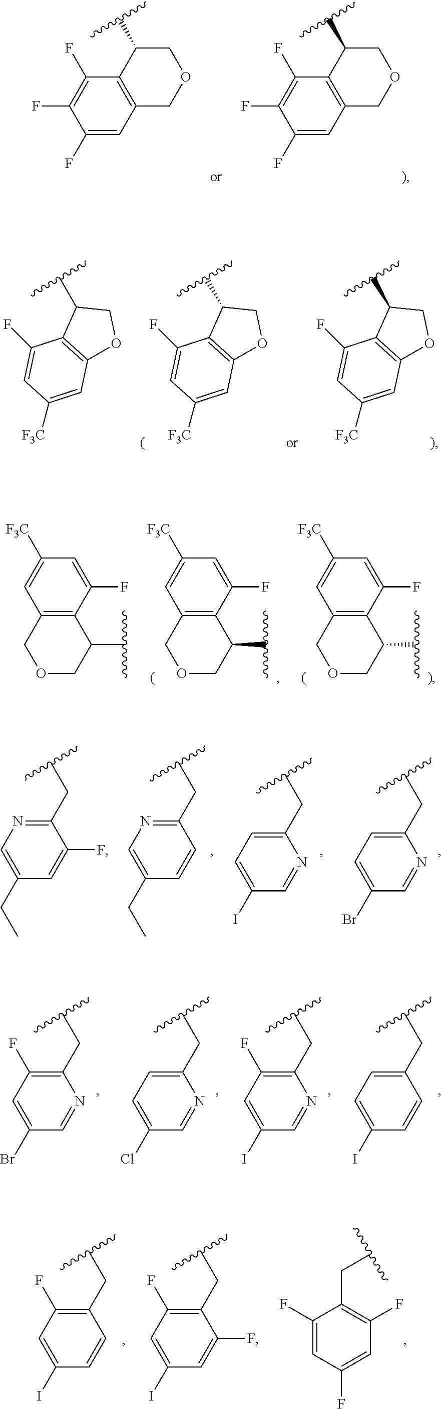

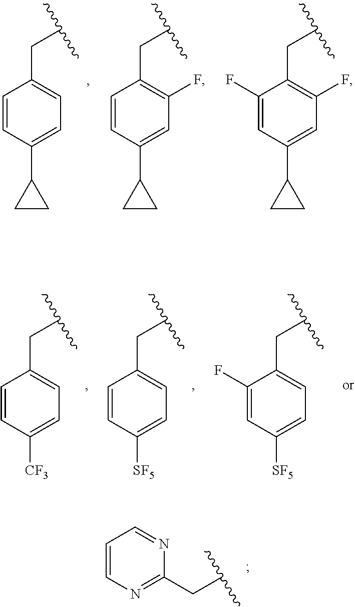

or
R⁹ and R¹⁰ together with the nitrogen atom to which they are attached form

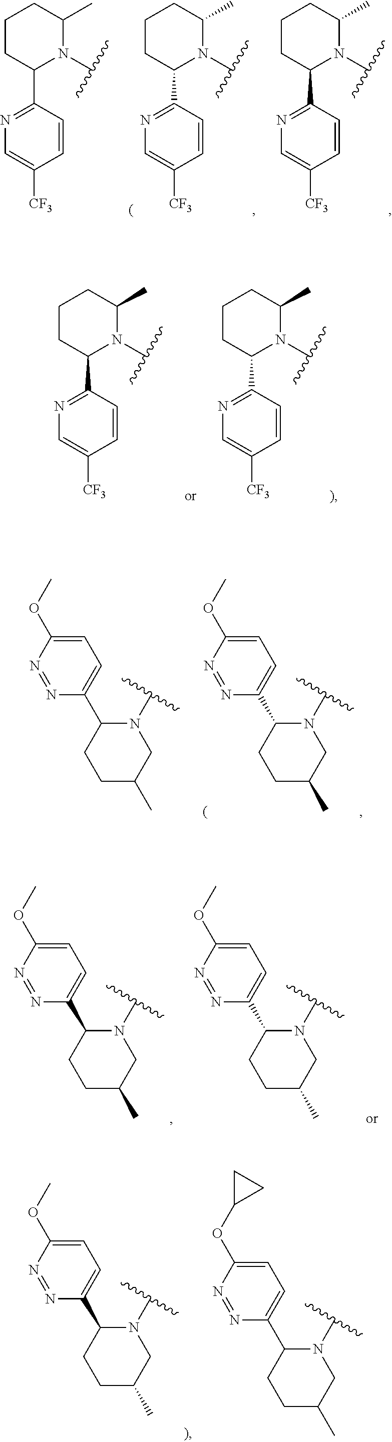

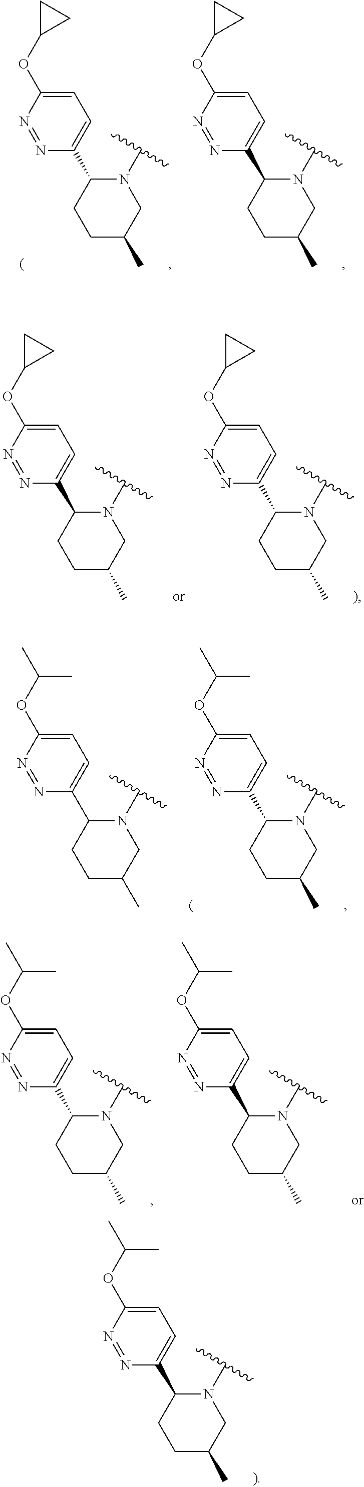

Aspect 14. The compound of any one of the preceding Aspects, wherein the compound is selected from

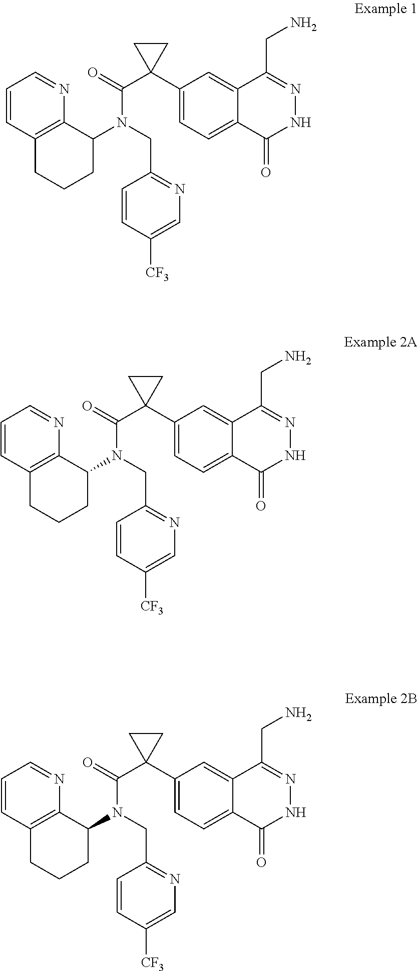 [SMILES: NCc1n[nH]c(=O)c2ccc(C3(C(=O)N(Cc4ccc(C(F)(F)F)cn4)C4CCCc5cccnc54)CC3)cc12 ; NCc1n[nH]c(=O)c2ccc(C3(C(=O)N(Cc4ccc(C(F)(F)F)cn4)[C@@H]4CCCc5cccnc54)CC3)cc12 ; NCc1n[nH]c(=O)c2ccc(C3(C(=O)N(Cc4ccc(C(F)(F)F)cn4)[C@H]4CCCc5cccnc54)CC3)cc12]

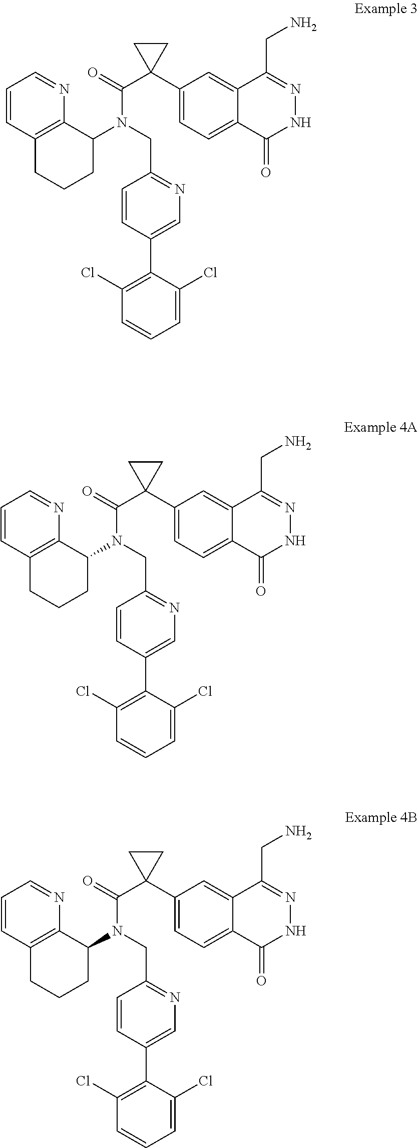 [SMILES: NCc1n[nH]c(=O)c2ccc(C3(C(=O)N(Cc4ccc(-c5c(Cl)cccc5Cl)cn4)C4CCCc5cccnc54)CC3)cc12 ; NCc1n[nH]c(=O)c2ccc(C3(C(=O)N(Cc4ccc(-c5c(Cl)cccc5Cl)cn4)[C@@H]4CCCc5cccnc54)CC3)cc12 ; NCc1n[nH]c(=O)c2ccc(C3(C(=O)N(Cc4ccc(-c5c(Cl)cccc5Cl)cn4)[C@H]4CCCc5cccnc54)CC3)cc12]

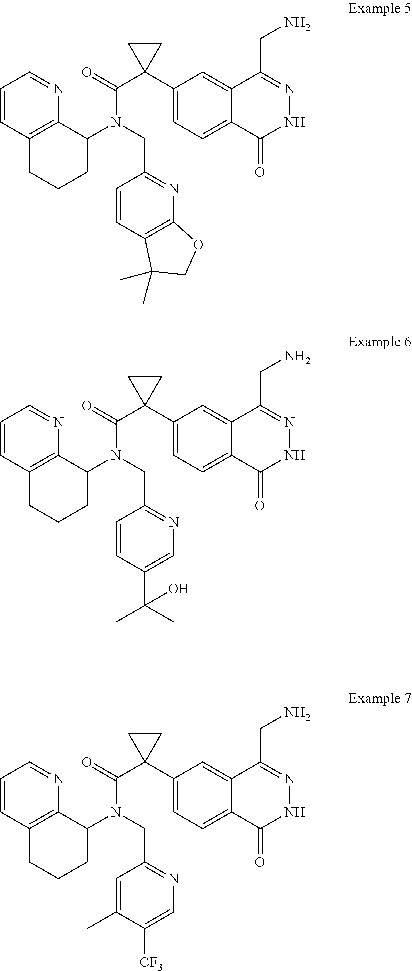 [SMILES: CC1(C)COc2nc(CN(C(=O)C3(c4ccc5c(=O)[nH]nc(CN)c5c4)CC3)C3CCCc4cccnc43)ccc21 ; CC(C)(O)c1ccc(CN(C(=O)C2(c3ccc4c(=O)[nH]nc(CN)c4c3)CC2)C2CCCc3cccnc32)nc1 ; Cc1cc(CN(C(=O)C2(c3ccc4c(=O)[nH]nc(CN)c4c3)CC2)C2CCCc3cccnc32)ncc1C(F)(F)F]

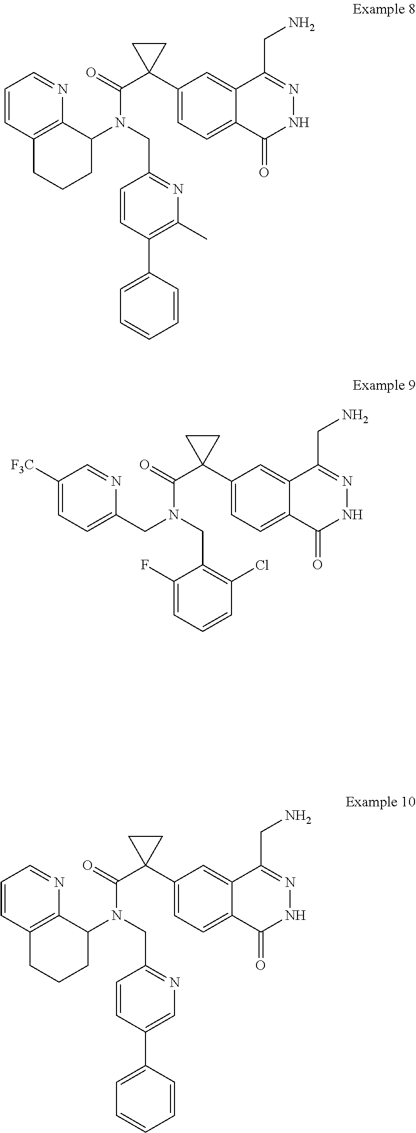 [SMILES: Cc1nc(CN(C(=O)C2(c3ccc4c(=O)[nH]nc(CN)c4c3)CC2)C2CCCc3cccnc32)ccc1-c1ccccc1 ; NCc1n[nH]c(=O)c2ccc(C3(C(=O)N(Cc4ccc(C(F)(F)F)cn4)Cc4c(F)cccc4Cl)CC3)cc12 ; NCc1n[nH]c(=O)c2ccc(C3(C(=O)N(Cc4ccc(-c5ccccc5)cn4)C4CCCc5cccnc54)CC3)cc12]

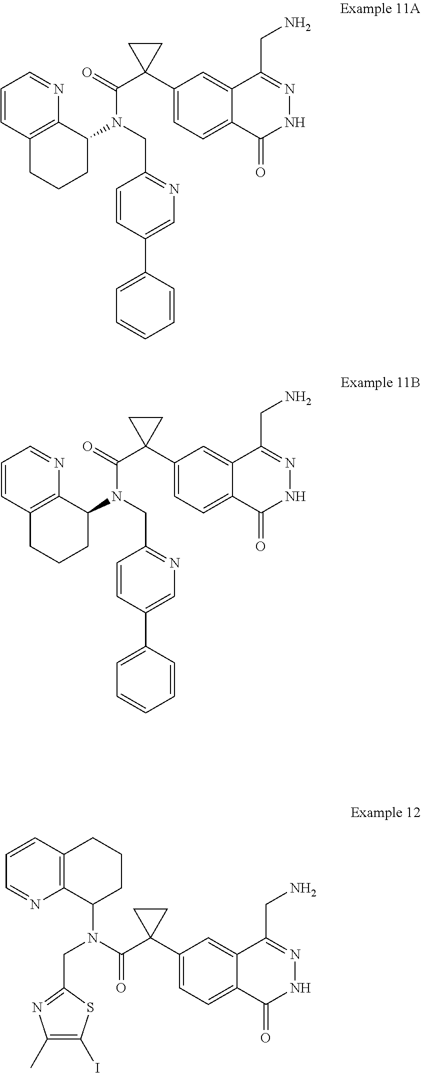 [SMILES: NCc1n[nH]c(=O)c2ccc(C3(C(=O)N(Cc4ccc(-c5ccccc5)cn4)[C@@H]4CCCc5cccnc54)CC3)cc12 ; NCc1n[nH]c(=O)c2ccc(C3(C(=O)N(Cc4ccc(-c5ccccc5)cn4)[C@H]4CCCc5cccnc54)CC3)cc12 ; Cc1nc(CN(C(=O)C2(c3ccc4c(=O)[nH]nc(CN)c4c3)CC2)C2CCCc3cccnc32)sc1I]

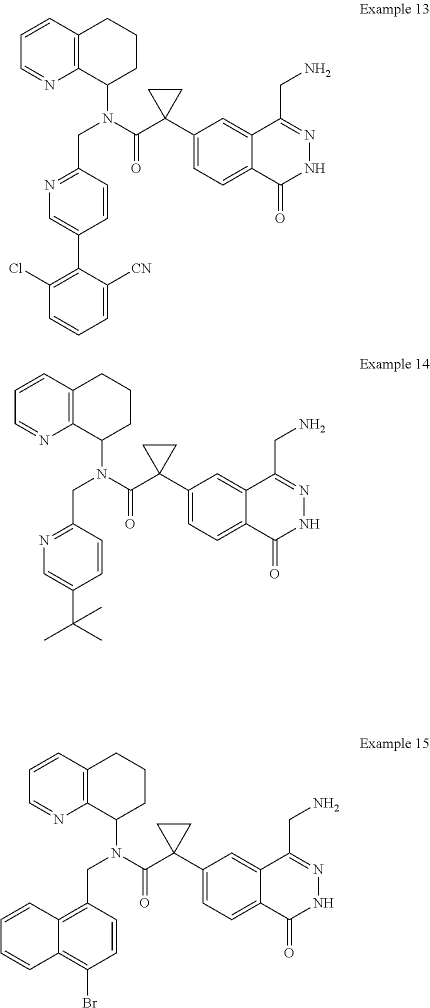

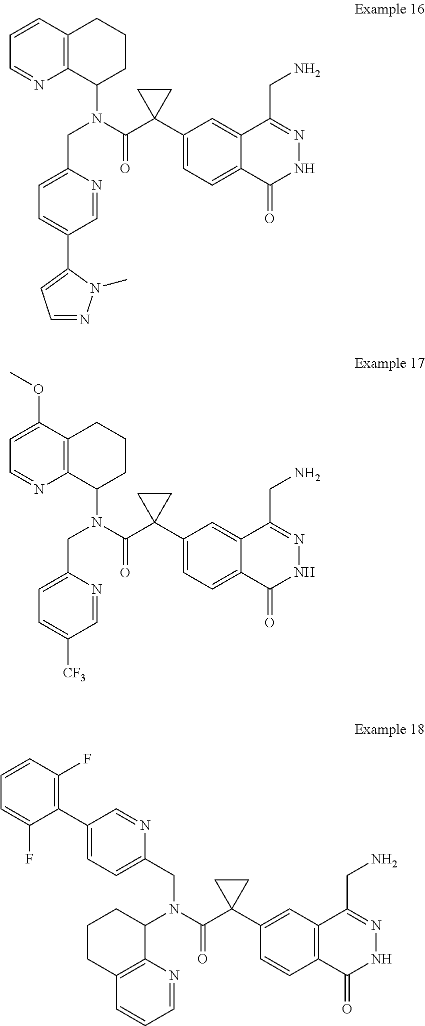 [SMILES: Cn1nccc1-c1ccc(CN(C(=O)C2(c3ccc4c(=O)[nH]nc(CN)c4c3)CC2)C2CCCc3cccnc32)nc1 ; COc1ccnc2c1CCCC2N(Cc1ccc(C(F)(F)F)cn1)C(=O)C1(c2ccc3c(=O)[nH]nc(CN)c3c2)CC1 ; NCc1n[nH]c(=O)c2ccc(C3(C(=O)N(Cc4ccc(-c5c(F)cccc5F)cn4)C4CCCc5cccnc54)CC3)cc12]

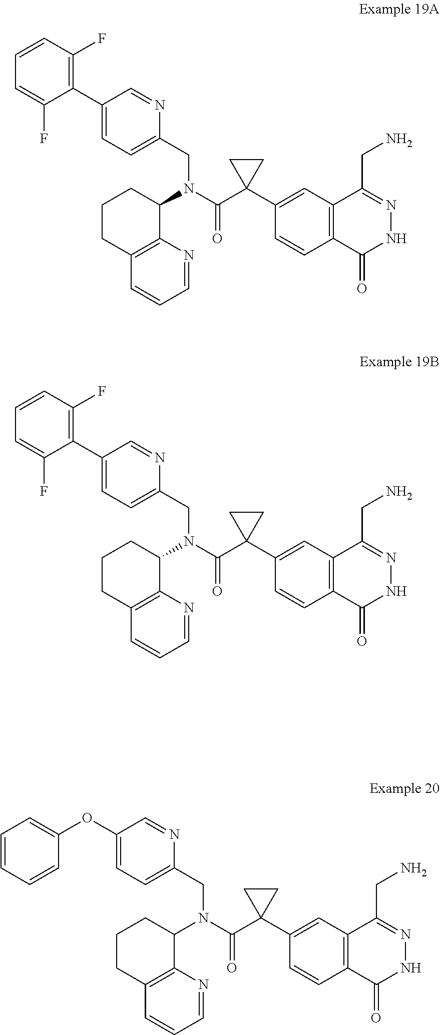 [SMILES: NCc1n[nH]c(=O)c2ccc(C3(C(=O)N(Cc4ccc(-c5c(F)cccc5F)cn4)[C@@H]4CCCc5cccnc54)CC3)cc12 ; NCc1n[nH]c(=O)c2ccc(C3(C(=O)N(Cc4ccc(-c5c(F)cccc5F)cn4)[C@H]4CCCc5cccnc54)CC3)cc12 ; NCc1n[nH]c(=O)c2ccc(C3(C(=O)N(Cc4ccc(Oc5ccccc5)cn4)C4CCCc5cccnc54)CC3)cc12]

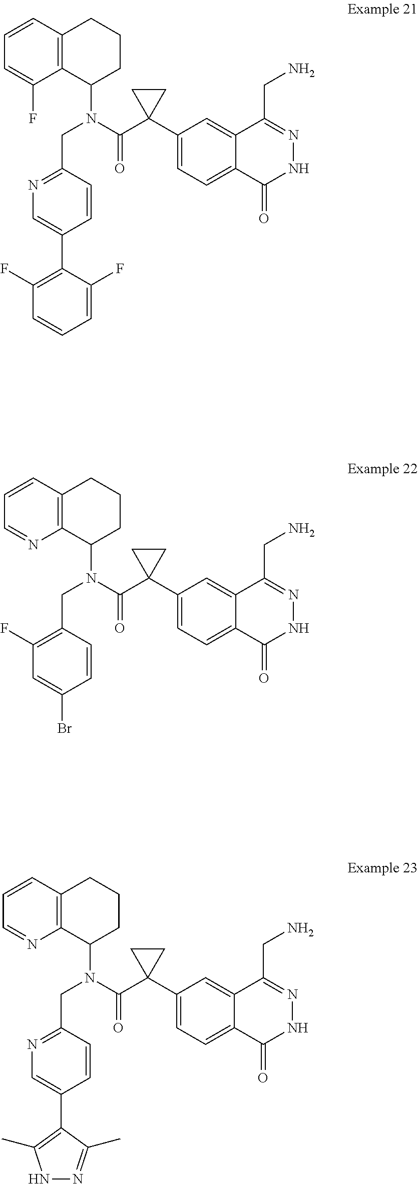 [SMILES: NCc1n[nH]c(=O)c2ccc(C3(C(=O)N(Cc4ccc(-c5c(F)cccc5F)cn4)C4CCCc5cccc(F)c54)CC3)cc12 ; NCc1n[nH]c(=O)c2ccc(C3(C(=O)N(Cc4ccc(Br)cc4F)C4CCCc5cccnc54)CC3)cc12 ; Cc1n[nH]c(C)c1-c1ccc(CN(C(=O)C2(c3ccc4c(=O)[nH]nc(CN)c4c3)CC2)C2CCCc3cccnc32)nc1]

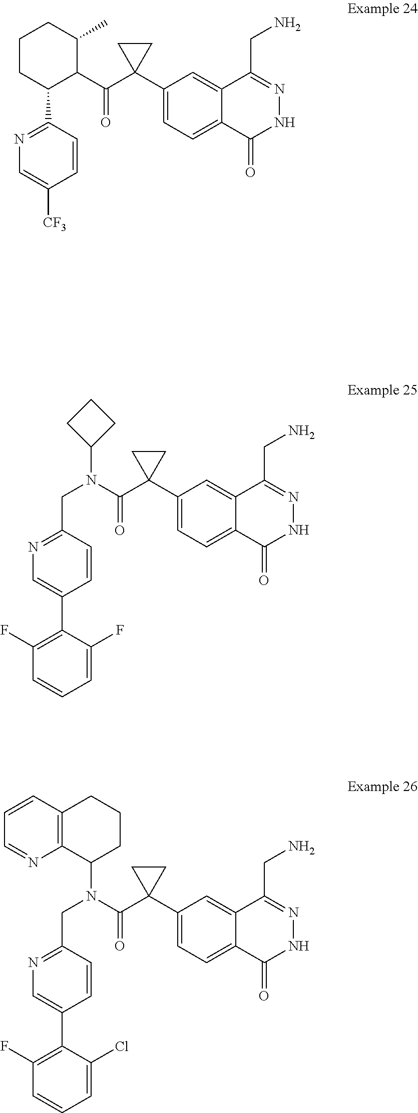 [SMILES: C[C@H]1CCC[C@@H](c2ccc(C(F)(F)F)cn2)C1C(=O)C1(c2ccc3c(=O)[nH]nc(CN)c3c2)CC1 ; NCc1n[nH]c(=O)c2ccc(C3(C(=O)N(Cc4ccc(-c5c(F)cccc5F)cn4)C4CCC4)CC3)cc12 ; NCc1n[nH]c(=O)c2ccc(C3(C(=O)N(Cc4ccc(-c5c(F)cccc5Cl)cn4)C4CCCc5cccnc54)CC3)cc12]

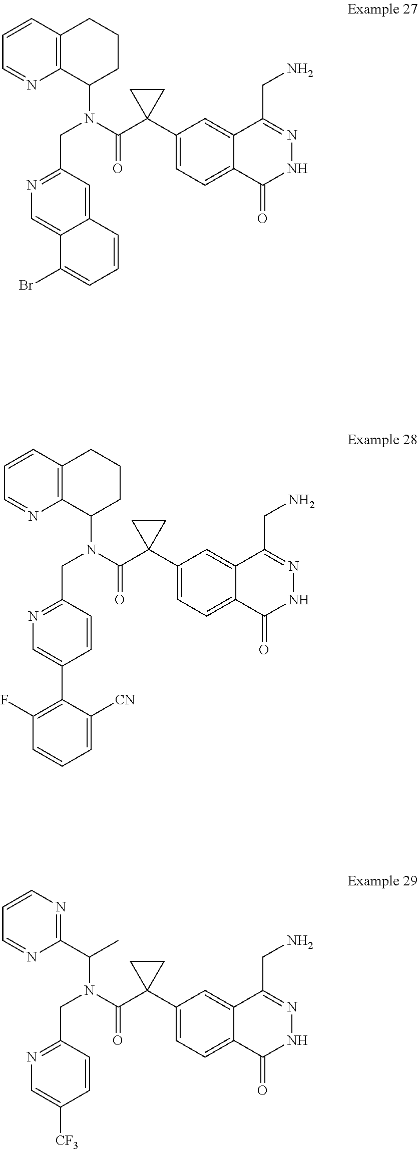 [SMILES: NCc1n[nH]c(=O)c2ccc(C3(C(=O)N(Cc4cc5cccc(Br)c5cn4)C4CCCc5cccnc54)CC3)cc12 ; N#Cc1cccc(F)c1-c1ccc(CN(C(=O)C2(c3ccc4c(=O)[nH]nc(CN)c4c3)CC2)C2CCCc3cccnc32)nc1 ; CC(c1ncccn1)N(Cc1ccc(C(F)(F)F)cn1)C(=O)C1(c2ccc3c(=O)[nH]nc(CN)c3c2)CC1]

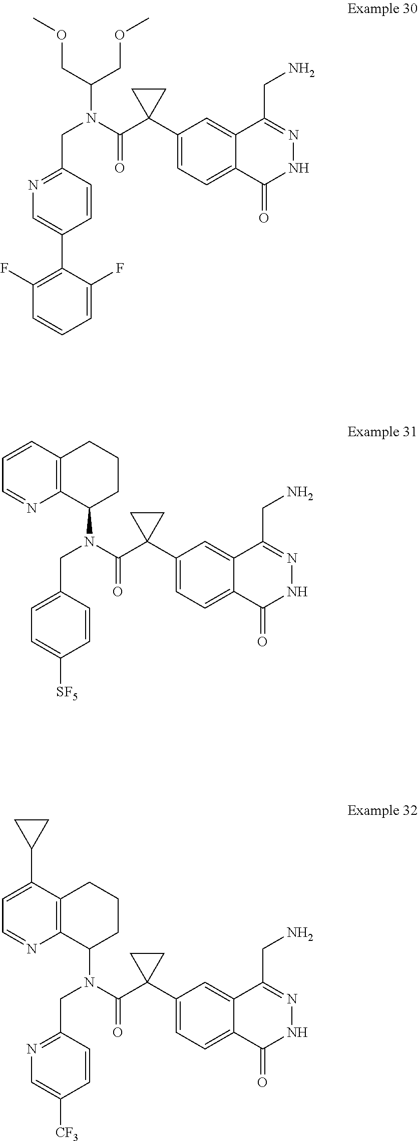

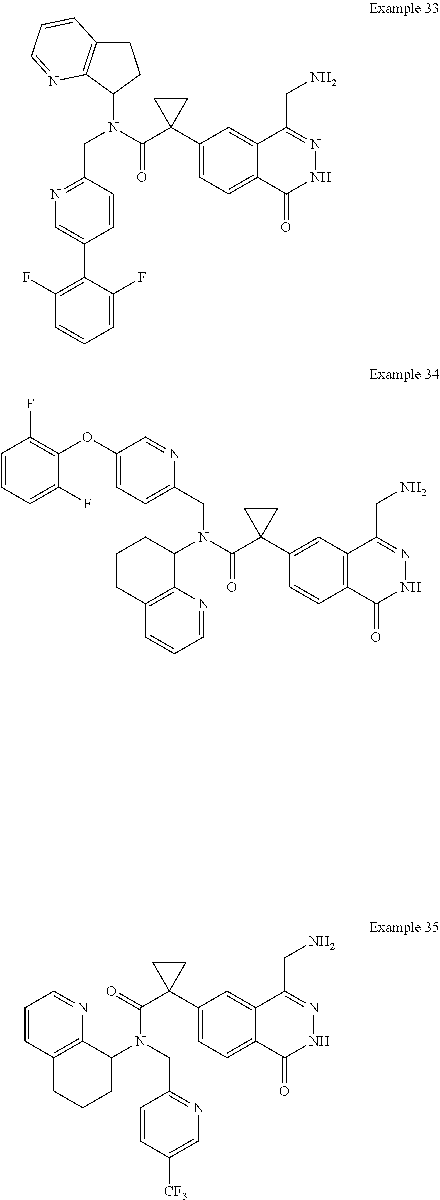 [SMILES: NCc1n[nH]c(=O)c2ccc(C3(C(=O)N(Cc4ccc(-c5c(F)cccc5F)cn4)C4CCc5cccnc54)CC3)cc12 ; NCc1n[nH]c(=O)c2ccc(C3(C(=O)N(Cc4ccc(Oc5c(F)cccc5F)cn4)C4CCCc5cccnc54)CC3)cc12 ; NCc1n[nH]c(=O)c2ccc(C3(C(=O)N(Cc4ccc(C(F)(F)F)cn4)C4CCCc5cccnc54)CC3)cc12]

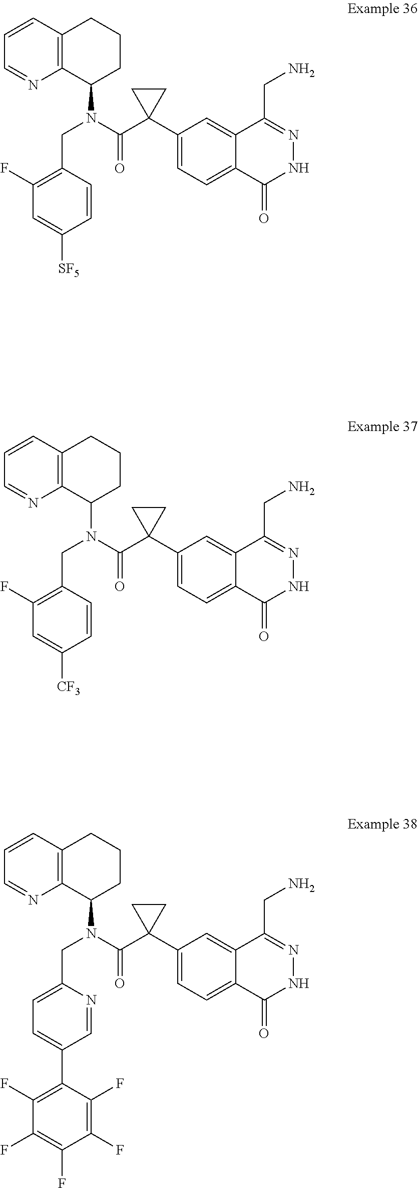

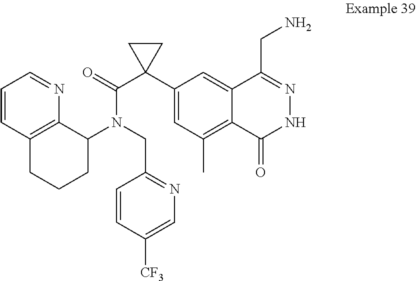 [SMILES: Cc1cc(C2(C(=O)N(Cc3ccc(C(F)(F)F)cn3)C3CCCc4cccnc43)CC2)cc2c(CN)n[nH]c(=O)c12]

Aspect 15. A pharmaceutical composition comprising a compound of any one of Aspects 1-14 or a pharmaceutically acceptable salt, stereoisomer, tautomer or prodrug thereof, together with a pharmaceutically acceptable excipient.

Aspect 16. A method of decreasing PRMT5 activity by inhibition, which comprises administering to an individual the compound according to any one of Aspects 1-14, or a pharmaceutically acceptable salt thereof, including the compound of formula (I) or the specific compounds exemplified herein.

Aspect 17. The method of Aspect 16, wherein the disease is selected from cancer.

Aspect 18. Use of a compound of any one of Aspects 1-14 or a pharmaceutically acceptable salt, stereoisomer, tautomer or prodrug thereof in the preparation of a medicament for treating a disease that is modulated by PRMT5.

Aspect 19. The use of Aspect 18, wherein the disease is cancer.

Aspect 20. The use of Aspect 18, wherein the disease is MTAP-null solid tumor, including but not limited to lung cancer, bladder cancer, melanoma, pancreatic cancer, esophageal cancer, gastric adenocarcinoma, breast cancer, glioblastoma, etc.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

In some embodiments, the structure

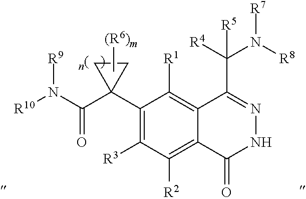

can turn into structure

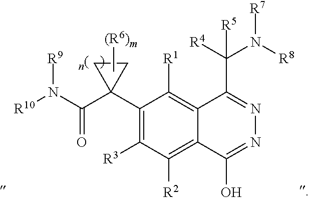

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C_{1_8}, C₁₋₆, and the like.

Unless specifically defined elsewhere in this document, all other technical and scientific terms used herein have the meaning commonly understood by one of ordinary skill in the art to which this invention belongs.

### EXAMPLES

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carded out, e.g., temperatures which can range from the solvent's boiling temperature. A given reaction can be carried out in one solvent or mixture of solvents.

The selection of appropriate protecting group, can be readily determined by one skilled in the art. In the synthesis schemes, some protection/deprotection steps are not shown and can be incorporated before, after or in between any steps. The protecting group shown in the synthesis schemes may or may not be used based on reaction conditions. The sequences of reactions may vary and provide similar results.

Reactions can be monitored according to any suitable method known in the art, such as NMR, UV, HPLC, LC-MS and TLC. Compounds can be purified by a variety of methods, including prep-HPLC and silica gel chromatography. Unless specified, prep-HPLC uses a buffered acetonitrile/water systems and silica gel chromatography (including column chromatography and prep-TLC) uses PE/EtOAc or DCM/MeOH systems as mobile phases. NMR spectra are recorded using a Bruker or Varian instrument with preset pulse sequences.

### ABBREVIATIONS

| | |
|---|---|
| NMR | nuclear magnetic resonance |
| UV | ultraviolet |
| HPLC | high performance liquid chromatography |
| prep | preparation/preparative |
| LC-MS | liquid chromatograph mass spectrometer |
| TLC | thin layer chromatography |
| PE | petroleum ether |
| Et | ethyl |
| Ac | acetyl |
| dppf | 1,1′-bis(diphenylphosphino)ferrocene |
| Me | methyl |
| DCM | dichloromethane |
| Boc | tert-butyloxycarbonyl |
| NBS | N-bromosuccinimide |
| Bu | butyl |
| Tf | trifluoromethanesulfonyl |
| dba | dibenzylideneacetone |
| BPD | bis(pinacolato)diboron |
| DMSO | dimethyl sulfoxide |
| XPhos | 2-(dicyclohexylphosphino)-2′,4′,6′-triisopropylbiphenyl |
| THF | tetrahydrofuran |
| Ph | phenyl |
| DMF | N,N-dimethylformamide |
| DIPEA | diisopropylethylamine |
| HATU | 1-[bis (dimethylamino)methylene]-1H-1,2,3-triazolo [4,5-b]pyridinium |
| | 3-oxid hexafluorophosphate |
| BOPCl | bis(2-oxo-3-oxazolidinyl)phosphinic chloride |
| DMAP | 4-dimethylaminopyridine |
| MTBE | methyl tert-butyl ether |

### Example 1: 1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclopropane-1-carboxamide

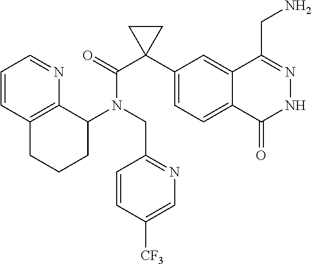 [SMILES: NCc1n[nH]c(=O)c2ccc(C3(C(=O)N(Cc4ccc(C(F)(F)F)cn4)C4CCCc5cccnc54)CC3)cc12]

### Step 1: 5-bromo-3-((dimethylamino)methylene)isobenzofuran-1(3H)-one

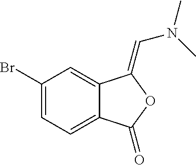

To a solution of 5-bromo-3H-isobenzofuran-1-one (100 g, 469 mmol) in toluene (600 mL) was added 1-tert-butoxy-N,N,N′,N′-tetramethyl-methanediamine (98.1 g, 563 mmol). The mixture was stirred at 90° C. for 12 h. The mixture was cooled to room temperature and solid was collected by filtration. Solid was rinsed with EtOAc (100 mL) and dried under vacuum to give the title compound (80.0 g, 64%). ¹H NMR (400 MHz, DMSO-d6) δ 7.97 (d, J=0.8 Hz, 1H), 7.60 (d, J=8.4 Hz, 1H), 7.29 (dd, J=1.4, 8.4 Hz, 1H), 7.00 (s, 1H), 3.10 (s, 6H).

### Step 2: 6-bromo-4-((dimethylamino)methyl)phthalazin-1(2H)-one

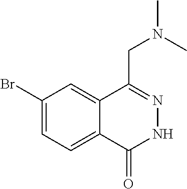 [SMILES: CN(C)Cc1n[nH]c(=O)c2ccc(Br)cc12]

To a solution of 5-bromo-3-((dimethylamino)methylene)isobenzofuran-1(3H)-one (80.0 g, 298 mmol) in EtOH (480 mL) was added hydrazine monohydrate (29.0 g, 579 mmol). The mixture was stirred at 80° C. for 12 h. The mixture was cooled to room temperature and solid was collected by filtration. Solid was dried under vacuum to give the title compound (70.0 g, 83%). LC-MS (M+H)⁺=282.0.

### Step 3: 6-bromo-4-(chloromethyl)phthalazin-1(2H)-one

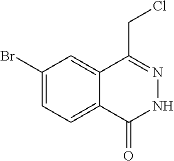 [SMILES: O=c1[nH]nc(CCl)c2cc(Br)ccc12]

To a solution of 6-bromo-4-((dimethylamino)methyl)phthalazin-1(2H)-one (60.0 g, 212 mmol) in anhydrous THF (360 mL) was added isobutyl chloroformate (116 g, 850 mmol) at 0-5° C. dropwise under nitrogen. Solid was collected by filtration and dried under vacuum to give the title compound (36.0 g, 62%).

### Step 4: 2-((7-bromo-4-oxo-3,4-dihydrophthalazin-1-yl)methyl)isoindoline-1,3-dione

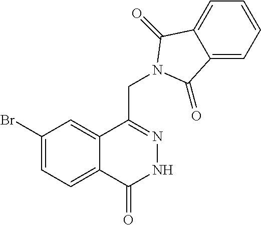 [SMILES: O=C1c2ccccc2C(=O)N1Cc1n[nH]c(=O)c2ccc(Br)cc12]

To a solution of 6-bromo-4-(chloromethyl)phthalazin-1(2H)-one (31.0 g, 113 mmol) in DMF (180 mL) was added potassium phthalate (31.4 g, 154 mmol). The mixture was stirred at 25° C. for 12 h. Solid was collected by filtration and dried under vacuum to give the title compound (40.0 g, 92%).

### Step 5: 4-(aminomethyl)-6-bromophthalazin-1(2H)-one

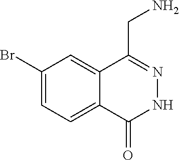 [SMILES: NCc1n[nH]c(=O)c2ccc(Br)cc12]

To a solution of 2-((7-bromo-4-oxo-3,4-dihydrophthalazin-1-yl)methyl)isoindoline-1,3-dione (40.0 g, 104 mmol) in EtOH (210 mL) was added hydrazine monohydrate (53.1 g, 1.04 mol). The mixture was stirred at 80° C. for 12 h. The mixture was cooled to room temperature and solid was collected by filtration. Solid was dried under vacuum to give the title compound (20.0 g, 76%). ¹H NMR (400 MHz, DMSO-d6) δ 8.37 (d, J=1.6 Hz, 1H), 8.18 (d, J=8.4 Hz, 1H), 8.12-8.06 (m, 2H), 8.03 (dd, J=1.8, 8.6 Hz, 1H), 7.88-7.81 (m, 2H), 4.00 (s, 2H).

### Step 6: tert-butyl ((7-bromo-4-oxo-3,4-dihydrophthalazin-1-yl)methyl)carbamate

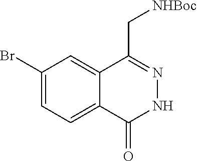 [SMILES: CC(C)(C)OC(=O)NCc1n[nH]c(=O)c2ccc(Br)cc12]

To a solution of 4-(aminomethyl)-6-bromophthalazin-1(2H)-one (20.0 g, 78.7 mmol) in DCM (144 mL) was added Boc₂O (34.3 g, 157 mmol) and triethylamine (23.9 g, 236 mmol). The mixture was stirred at 20° C. for 12 h. Solid was collected by filtration and dried under vacuum to give the title compound (20.0 g, 72%). ¹H NMR (400 MHz, DMSO-d6) δ 12.7 (br s, 1H), 8.28 (s, 1H), 8.18 (d, J=8.2 Hz, 1H), 8.04 (d, J=8.2 Hz, 1H), 7.48 (br s, 1H), 5.78 (s, 1H), 4.53-4.31 (m, 2H), 1.42 (s, 10H).

### Step 7: ethyl 1-(4-(((tert-butoxycarbonyl)amino)methyl)-1-oxo-1,2-dihydrophthalazin-6-yl)cyclopropane-1-carboxylate

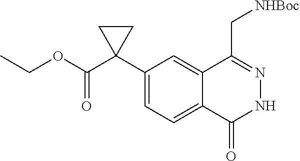 [SMILES: CCOC(=O)C1(c2ccc3c(=O)[nH]nc(CNC(=O)OC(C)(C)C)c3c2)CC1]

To a suspension of zinc dust (9.9 g, 154.6 mmol) in anhydrous THF (70 mL) at rt was added 1,2-dibromoethane (1.94 g, 10.3 mmol) under nitrogen. The reaction mixture was stirred at 65° C. for 5 min and cooled to room temperature. This activation process was repeated for 3 times. Chlorotrimethylsilane (0.559 g, 5.18 mmol) was added and the resulting suspension was stirred at room temperature for 15 min. The mixture was warmed to 65° C. and solution of ethyl 1-bromocyclopropane-1-carboxylate (10.0 g, 51.8 mmol) in anhydrous THF (30 mL) was added at a rate that reflux was maintained. The reaction mixture was refluxed for an additional 5 h and cooled to room temperature. Stirring was stopped to allow settling of solid. The supernatant was syringed into a solution of tert-butyl ((7-bromo-4-oxo-3,4-dihydrophthalazin-1-yl)methyl)carbamate (10.0 g, 28.3 mmol) in THF (100 mL) followed by addition of Pd₂(dba)₃ (1.29 g, 1.41 mmol) and Qphos (2.0 g, 2.83 mmol). The mixture was stirred for 16 h at 40° C. under nitrogen. The mixture was cooled to room temperature and filtered. The filtrate was concentrated under vacuum, and the filter cake was rinsed with EtOAc (200 mL). Combined filtrate was concentrated under vacuum. The residue was purified by silica gel chromatograph (PE:EtOAc=1:1) to give the title compound (8.0 g, 73%). LC-MS (M+H)⁺=388.3.

### Step 8: 1-(4-(((tert-butoxycarbonyl)amino)methyl)-1-oxo-1,2-dihydrophthalazin-6-yl)cyclopropane-1-carboxylic acid

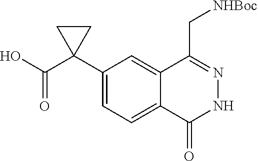 [SMILES: CC(C)(C)OC(=O)NCc1n[nH]c(=O)c2ccc(C3(C(=O)O)CC3)cc12]

To a solution of ethyl 1-(4-(((tert-butoxycarbonyl)amino)methyl)-1-oxo-1,2-dihydrophthalazin-6-yl)cyclopropane-1-carboxylate (8.0 g, 20.6 mmol) in THF (60 mL), water (20 mL) and MeOH (20 mL) was added LiOH (2.6 g, 62 mol). The mixture was stirred for 16 h at 60° C. and cooled to room temperature. The reaction mixture was cooled to room temperature, concentrated under vacuum, diluted into water (150 mL) and extracted with EtOAc (150 mL×2). The organic layer was discarded and the aqueous later was acidified with citric acid. The mixture was extracted with EtOAc (150 mL×2). The combined organic layer was washed with brine (100 mL), dried over Na₂SO₄, filtered and concentrated under vacuum to give the title compound (4.0 g, 54%). ¹H NMR (500 MHz, DMSO-d6) δ 12.53 (s, 2H), 8.23-8.14 (m, 1H), 7.99-7.76 (m, 2H), 7.46-7.34 (m, 1H), 4.55-4.34 (m, 2H), 1.61-1.46 (m, 2H), 1.47-1.31 (m, 9H), 1.32-1.23 (m, 2H). LC-MS (M+H)⁺=360.1.

### Step 9: N-((5-(trifluoromethyl)pyridin-2-yl)methyl)-5,6,7,8-tetrahydroquinolin-8-amine

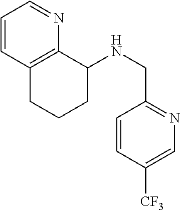 [SMILES: FC(F)(F)c1ccc(CNC2CCCc3cccnc32)nc1]

To a solution 5,6,7,8-tetrahydroquinolin-8-amine (0.30 g, 2.02 mmol) in MeOH (5 mL) was added 5-(trifluoromethyl)picolinaldehyde (354 mg, 2.02 mmol), AcOH (12 mg, 0.202 mmol) at 15° C. with stirring. After 1 h, NaBH₃CN (153 mg, 2.43 mmol) was added and the mixture was stirred at 15° C. for 12 h. The mixture was concentrated under reduced pressure and the residue was purified by silica gel chromatograph (PE/EtOAc=1/0 to 0/1) to give the title compound (98 mg, 16%). ¹H NMR (400 MHz, CDCl₃) δ 8.84 (s, 1H), 8.43 (d, J=4.0 Hz, 1H), 7.89 (dd, J=8.4, 2.2 Hz, 1H), 7.63 (d, J=8.0 Hz, 1H), 7.40 (d, J=8.0 Hz, 1H), 7.10 (dd, J=8.4, 4.8 Hz, 1H), 4.19 (s, 2H), 3.93-3.85 (m, 1H), 2.90-2.73 (m, 2H), 2.21-2.14 (m, 1H), 2.07-1.98 (m, 2H), 1.84-1.68 (m, 3H). LC-MS (M+H)⁺=308.1.

### Step 10: tert-butyl ((4-oxo-7-(1-((5,6,7,8-tetrahydroquinolin-8-yl)((5-(trifluoromethyl)pyridin-2-yl)methyl)carbamoyl)cyclopropyl)-3,4-dihydrophthalazin-1-yl)methyl)carbamate

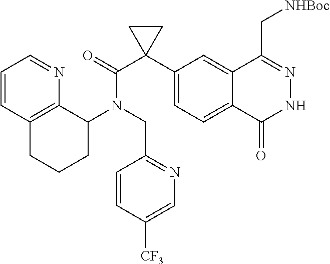 [SMILES: CC(C)(C)OC(=O)NCc1n[nH]c(=O)c2ccc(C3(C(=O)N(Cc4ccc(C(F)(F)F)cn4)C4CCCc5cccnc54)CC3)cc12]

To a solution of 1-(4-(((tert-butoxycarbonyl)amino)methyl)-1-oxo-1,2-dihydrophthalazin-6-yl)cyclopropane-1-carboxylic acid (55 mg, 0.15 mmol) in DMF (2.5 mL) was added HATU (64 mg, 0.17 mmol), N-((5-(trifluoromethyl)pyridin-2-yl)methyl)-5,6,7,8-tetrahydroquinolin-8-amine (51.7 mg, 0.17 mmol) and DIPEA (39.5 mg, 0.31 mmol). The solution was stirred for 3 h at 50° C. The mixture was cooled to room temperature, diluted with water (100 mL) and then extracted with EtOAc (100 mL). The combined organic layer was washed with brine (100 mL×2), dried over Na₂SO₄, filtered and concentrated under vacuum. The residue was purified by silica gel chromatograph (DCM:MeOH=20:1) to give the title compound (43 mg, 44%). LC-MS (M+H)⁺=649.2.

### Step 11: 1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclopropane-1-carboxamide

To a solution of tert-butyl ((4-oxo-7-(1-((5,6,7,8-tetrahydroquinolin-8-yl)((5-(trifluoromethyl)pyridin-2-yl)methyl)carbamoyl)cyclopropyl)-3,4-dihydrophthalazin-1-yl)methyl)carbamate (43 mg, 0.07 mmol) in DCM (4 mL) was added TFA (1 mL). The mixture was stirred for 1 h at room temperature then concentrated under vacuum. The residue was purified by prep-HPLC to give Example 1 (20 mg, 55%). ¹H NMR (500 MHz, DMSO-d6) δ 12.55-12.33 (m, 1H), 8.96-8.57 (m, 1H), 8.50-7.40 (m, 7H), 7.29-7.12 (m, 1H), 5.60-5.46 (m, 1H), 5.01-3.58 (m, 4H), 2.84-2.53 (m, 2H), 2.25-1.29 (m, 8H), 1.13-0.88 (m, 2H). LC-MS (M+H)⁺=549.3.

### Example 2A & 2B: (R)-1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclopropane-1-carboxamide & (S)-1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclopropane-1-carboxamide

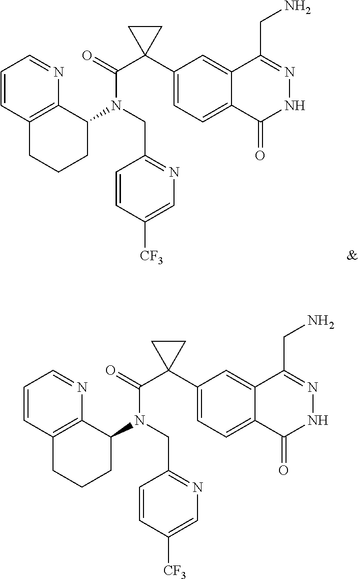 [SMILES: NCc1n[nH]c(=O)c2ccc(C3(C(=O)N(Cc4ccc(C(F)(F)F)cn4)[C@@H]4CCCc5cccnc54)CC3)cc12 ; NCc1n[nH]c(=O)c2ccc(C3(C(=O)N(Cc4ccc(C(F)(F)F)cn4)[C@H]4CCCc5cccnc54)CC3)cc12]

Example 1 (20 mg) was separated by chiral prep-HPLC to give Example 2A (7 mg, 35%) and Example 2B (5 mg, 25%). Analytical chiral-HPLC condition as below. Column: CHIRALPAK IC-3; Column size: 4.6×50 mm, 3 m; Mobile phase: (Hexane:DCM=3:1, with 0.1% diethylamine):EtOH=70:30; Flow: 1 mL/min; Temperature: 25° C.

Example 2A: Chiral HPLC t_{R}=3.29 min. ¹H NMR (500 MHz, DMSO) δ 12.79-12.42 (m, 1H), 8.96-8.56 (m, 1H), 8.50-7.40 (m, 7H), 7.26-7.16 (m, 1H), 5.65-5.28 (m, 1H), 5.03-3.62 (m, 4H), 2.80-2.59 (m, 2H), 2.22-1.36 (m, 8H), 1.00-0.79 (m, 2H). LC-MS (M+H)⁺=549.3

Example 2B: Chiral HPLC t_{R}=4.05 min. 1H NMR (500 MHz, DMSO) δ 12.55-12.33 (m, 1H), 8.96-8.57 (m, 1H), 8.50-7.40 (m, 7H), 7.25-7.15 (m, 1H), 5.62-5.29 (m, 1H), 5.04-3.56 (m, 4H), 2.82-2.62 (m, 2H), 2.25-1.29 (m, 8H), 1.13-0.80 (m, 2H). LC-MS (M+H)⁺=549.3.

### Example 3: 1-(4-(aminomethyl)-1-oxo-1,2-dihydrophthalazin-6-yl)-N-((5-(2,6-dichlorophenyl)pyridin-2-yl)methyl)-N-(5,6,7,8-tetrahydroquinolin-8-yl)cyclopropane-1-carboxamide

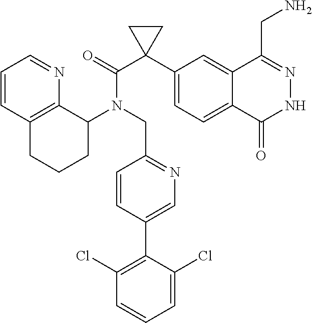 [SMILES: NCc1n[nH]c(=O)c2ccc(C3(C(=O)N(Cc4ccc(-c5c(Cl)cccc5Cl)cn4)C4CCCc5cccnc54)CC3)cc12]

### Step 1: tert-butyl ((5-(2,6-dichlorophenyl)pyridin-2-yl)methyl)carbamate

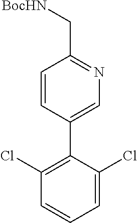 [SMILES: CC(C)(C)OC(=O)NCc1ccc(-c2c(Cl)cccc2Cl)cn1]

To a solution of tert-butyl ((5-bromopyridin-2-yl)methyl)carbamate (18.0 g, 62.7 mmol) and (2,6-dichlorophenyl)boronic acid (23.9 g, 125 mmol) in dioxane (80 mL), toluene (40 mL) and water (40 mL) was added K₃PO₄ (33.3 g, 157 mmol) and Pd(dppf)Cl₂ (4.59 g, 6.27 mmol) under nitrogen. The mixture was stirred at 85° C. for 12 h and cooled to room temperature. The mixture was diluted with water (150 mL) and extracted with EtOAc (80 mL×3). The combined organic layer was washed with brine (50 mL×2), dried over Na₂SO₄, filtered and concentrated under reduced pressure. The residue was purified by silica gel chromatography (PE:EtOAc=1:0 to 5:1) to give the title compound (9.0 g, 41%). LC-MS (M+H)⁺=353.1.

### Step 2: (5-(2,6-dichlorophenyl)pyridin-2-yl)methanamine

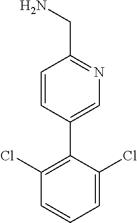 [SMILES: NCc1ccc(-c2c(Cl)cccc2Cl)cn1]

A mixture of tert-butyl ((5-(2,6-dichlorophenyl)pyridin-2-yl)methyl)carbamate (9.0 g, 25.5 mmol) in methanolic HCl (4 M, 90 mL) was stirred at 25° C. for 1 h, then the mixture was neutralized with methanolic NaOH (1 M) to pH 7. The mixture was concentrated to dryness under reduced pressure, and the residue was triturated with DCM/MeOH (10/1, 10 mL). The filtrate was collected by filtration and concentrated under vacuum to give the title compound (6.0 g, 93%). ¹H NMR (400 MHz, CDCl₃) δ 8.45 (s, 1H), 7.66-7.58 (m, 1H), 7.54 (d, J=8.0 Hz, 1H), 7.38 (d, J=8.0 Hz, 2H), 7.30-7.24 (m, 1H), 4.58 (s, 2H).

### Step 3: N-((5-(2,6-dichlorophenyl)pyridin-2-yl)methyl)-5,6,7,8-tetrahydroquinolin-8-amine

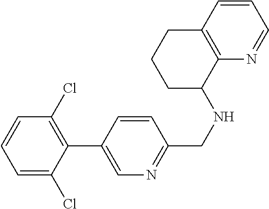 [SMILES: Clc1cccc(Cl)c1-c1ccc(CNC2CCCc3cccnc32)nc1]

To a solution of (5-(2,6-dichlorophenyl)pyridin-2-yl)methanamine (10.8 g, 42.8 mmol) and 6,7-dihydroquinolin-8(5H)-one (7.0 g, 47.6 mmol) in DCM (140 mL) and MeOH (1.4 mL) was added NaBH(OAc)₃ (18.1 g, 85.6 mmol). The mixture was stirred at 25° C. for 2 h. The mixture was poured into saturated NaHCO₃ (100 mL). The mixture was extracted with DCM (50 mL×3). The combined organic layer was washed with brine (30 mL), dried with Na₂SO₄, filtered and concentrated under vacuum. The residue was purified by silica gel chromatography (PE:EtOAc=1:0 to 0:1) to give the title compound (5.5 g, 30%). ¹H NMR (400 MHz, CD₃OD) δ 8.43-8.35 (m, 2H), 7.78-7.70 (m, 1H), 7.66 (d, J=8.0 Hz, 1H), 7.58-7.50 (m, 3H), 7.44-7.36 (m, 1H), 7.25-7.17 (m, 1H), 4.73-4.56 (m, 2H), 3.90 (dd, J=7.6, 6.4 Hz, 1H), 2.95-2.76 (m, 2H), 2.33-2.23 (m, 1H), 2.12-2.03 (m, 2H), 1.93-1.72 (m, 2H). LC-MS (M+H)⁺=384.0.

### Step 4: tert-butyl ((7-(1-(((5-(2,6-dichlorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

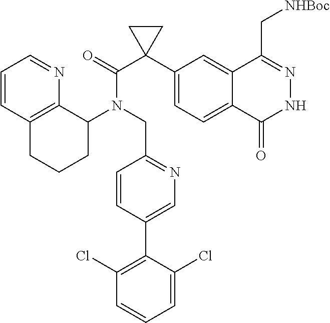 [SMILES: CC(C)(C)OC(=O)NCc1n[nH]c(=O)c2ccc(C3(C(=O)N(Cc4ccc(-c5c(Cl)cccc5Cl)cn4)C4CCCc5cccnc54)CC3)cc12]

To a solution of 1-(4-(((tert-butoxycarbonyl)amino)methyl)-1-oxo-1,2-dihydrophthalazin-6-yl)cyclopropane-1-carboxylic acid (55 mg, 0.15 mmol) in DMF (2.5 mL) was added HATU (64 mg, 0.17 mmol), N-((5-(2,6-dichlorophenyl)pyridin-2-yl)methyl)-5,6,7,8-tetrahydroquinolin-8-amine (64.3 mg, 0.17 mmol) and DIPEA (39.5 mg, 0.31 mmol). The mixture was stirred for 3 h at 50° C. The reaction mixture was cooled to room temperature, diluted with water (100 mL), and then extracted with EtOAc (100 mL). The combined organic layer was washed with brine (100 mL×2), dried over Na₂SO₄, filtered and concentrated under vacuum. The residue was purified by silica gel chromatograph (DCM:MeOH=20:1) to give the title compound (30 mg, 27%). LC-MS (M+H)⁺=725.3.

### Step 5: 1-(4-(aminomethyl)-1-oxo-1,2-dihydrophthalazin-6-yl)-N-((5-(2,6-dichlorophenyl)pyridin-2-yl)methyl)-N-(5,6,7,8-tetrahydroquinolin-8-yl)cyclopropane-1-carboxamide

To a solution of tert-butyl ((7-(1-(((5-(2,6-dichlorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate (30 mg, 0.04 mmol) in DCM (4 mL) was added TFA (1 mL). The mixture was stirred for 1 h at room temperature then concentrated under vacuum. The residue was purified by prep-HPLC to give Example 3. ¹H NMR (500 MHz, DMSO-d6) δ 12.55-12.33 (m, 1H), 8.57-8.00 (m, 4H), 7.93-7.82 (m, 1H), 7.81-7.69 (m, 1H), 7.67-7.55 (m, 2H), 7.55-7.41 (m, 2H), 7.39-7.30 (m, 1H), 7.27-7.13 (m, 1H), 5.68-4.00 (m, 4H), 3.70-3.55 (m, 1H), 2.87-2.60 (m, 2H), 2.10-1.38 (m, 8H), 1.12-0.68 (m, 2H). LC-MS (M+H)⁺=625.3.

### Example 4A & 4B: (R)-1-(4-(aminomethyl)-1-oxo-1,2-dihydrophthalazin-6-yl)-N-((5-(2,6-dichlorophenyl)pyridin-2-yl)methyl)-N-(5,6,7,8-tetrahydroquinolin-8-yl)cyclopropane-1-carboxamide & (S)-1-(4-(aminomethyl)-1-oxo-1,2-dihydrophthalazin-6-yl)-N-((5-(2,6-dichlorophenyl)pyridin-2-yl)methyl)-N-(5,6,7,8-tetrahydroquinolin-8-yl)cyclopropane-1-carboxamide

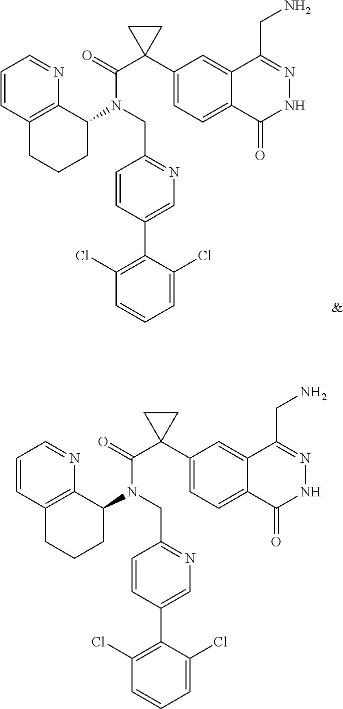 [SMILES: NCc1n[nH]c(=O)c2ccc(C3(C(=O)N(Cc4ccc(-c5c(Cl)cccc5Cl)cn4)[C@@H]4CCCc5cccnc54)CC3)cc12 ; NCc1n[nH]c(=O)c2ccc(C3(C(=O)N(Cc4ccc(-c5c(Cl)cccc5Cl)cn4)[C@H]4CCCc5cccnc54)CC3)cc12]

Example 3 (21 mg) was separated by chiral prep-HPLC to give Example 4A (7 mg, 33%) and Example 4B (7 mg, 33%). Analytical chiral-HPLC condition as below. Column: CHIRALPAK IC-3; Column size: 4.6×50 mm, 3 m; Mobile phase: (Hexane:DCM=3:1, with 0.1% diethylamine):EtOH=50:50; Flow: 1 mL/min; Temperature: 25° C.

Example 4A: Chiral HPLC t_{R}=3.32 min. ¹H NMR (500 MHz, DMSO-d6) δ 12.54-12.30 (m, 1H), 8.56-8.00 (m, 4H), 7.93-7.82 (m, 1H), 7.80-7.70 (m, 1H), 7.67-7.55 (m, 2H), 7.53-7.42 (m, 2H), 7.40-7.29 (m, 1H), 7.28-7.15 (m, 1H), 5.67-3.88 (m, 4H), 3.66-3.48 (m, 1H), 2.86-2.59 (m, 2H), 2.12-1.31 (m, 8H), 1.14-0.73 (m, 2H). LC-MS (M+H)⁺=625.3.

Example 4B: Chiral HPLC t_{R}=5.38 min. ¹H NMR (500 MHz, DMSO-d6) δ 12.55-12.34 (m, 1H), 8.57-7.99 (m, 4H), 7.93-7.82 (m, 1H), 7.81-7.69 (m, 1H), 7.67-7.55 (m, 2H), 7.55-7.41 (m, 2H), 7.39-7.30 (m, 1H), 7.27-7.15 (m, 1H), 5.68-4.00 (m, 4H), 3.71-3.54 (m, 1H), 2.87-2.62 (m, 2H), 2.10-1.38 (m, 8H), 1.12-0.65 (m, 2H). LC-MS (M+H)⁺=625.3.

### Example 5: 1-(4-(aminomethyl)-1-oxo-1,2-dihydrophthalazin-6-yl)-N-((3,3-dimethyl-2,3-dihydrofuro[2,3-b]pyridin-6-yl)methyl)-N-(5,6,7,8-tetrahydroquinolin-8-yl)cyclopropane-1-carboxamide

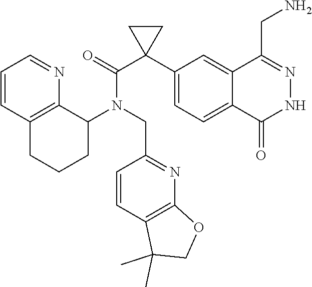 [SMILES: CC1(C)COc2nc(CN(C(=O)C3(c4ccc5c(=O)[nH]nc(CN)c5c4)CC3)C3CCCc4cccnc43)ccc21]

### Step 1: 3-iodo-2-((2-methylallyl)oxy)pyridine

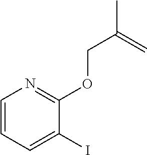

To a mixture of NaH (60%, 4.99 g, 125 mmol) in THF (450 mL) was added 2-methylprop-2-en-1-ol (4.5 g, 62.41 mmol) dropwise at 25° C. under nitrogen. The mixture was stirred at 25° C. for 10 min followed by addition of 2-fluoro-3-iodo-pyridine (13.9 g, 62.4 mmol). After 30 min, the mixture was diluted with ice water (400 mL). The mixture was extracted with EtOAc (100 mL×2). The combined organic layer was washed with brine (10 mL), dried over Na₂SO₄, filtered and concentrated under vacuum. The residue was purified by silica gel chromatograph (PE/EtOAc=1/0 to 10/1) to give the title compound (10.3 g, 60%). ¹H NMR (400 MHz, CDCl₃) δ 8.10 (dd, J=4.8, 1.6 Hz, 1H), 8.04 (dd, J=7.6, 1.6 Hz, 1H), 6.66 (dd, J=7.6, 4.8 Hz, 1H), 5.19-5.15 (m, 1H), 5.00-4.96 (m, 1H), 4.79 (s, 2H), 1.88 (s, 3H).

### Step 2: 3,3-dimethyl-2,3-dihydrofuro[2,3-b]pyridine

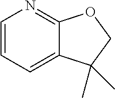

To a solution of 3-iodo-2-((2-methylallyl)oxy)pyridine (9.3 g, 33.8 mmol) in DMF (90 mL) was added HCOONa (2.30 g, 33.8 mmol), TBAC (9.40 g, 33.8 mmol), K₂CO₃ (14.0 g, 101 mmol) and Pd(OAc)₂ (759 mg, 3.38 mmol) under nitrogen. The mixture was warmed to 100° C. and stirred for 16 h. The mixture was cooled to room temperature and diluted with water (200 mL). The mixture was extracted with EtOAc (100 mL×3). The combined organic layer was washed with water (50 mL×2) and concentrated in vacuum. The residue was purified by silica gel chromatograph (PE/EtOAc=1/0 to 10/1) to give the title compound (2.5 g, 50%). ¹H NMR (400 MHz, CDCl₃) δ 8.01 (dd, J=5.2, 2.0 Hz, 1H), 7.39 (dd, J=7.0, 5.8 Hz, 1H), 6.81 (dd, J=7.2, 5.2 Hz, 1H), 4.27 (s, 2H), 1.37 (s, 6H).

### Step 3: 3,3-dimethyl-2,3-dihydrofuro[2,3-b]pyridine 7-oxide

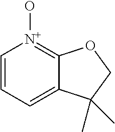

To a solution of 3,3-dimethyl-2,3-dihydrofuro[2,3-b]pyridine (2.5 g, 16.76 mmol) in DCM (50 mL) was added mCPBA (85%, 5.10 g, 25.1 mmol) at 0° C. The mixture was stirred at 25° C. for 12 h then quenched with 10% Na₂S₂O₃ (20 mL). The mixture was extracted with DCM (15 mL×12). The combined organic layer was concentrated under reduced pressure. The residue was purified by silica gel chromatograph (PE/EtOAc=1/1 to 0/1) to give the title compound (2.5 g, 91%). ¹H NMR (400 MHz, CDCl₃) δ 7.95 (dd, J=6.6, 5.0 Hz, 1H), 7.39-7.25 (m, 1H), 6.97-6.91 (m, 1H), 4.47 (s, 2H), 1.34 (s, 6H).

### Step 4: 3,3-dimethyl-2,3-dihydrofuro[2,3-b]pyridine-6-carbonitrile

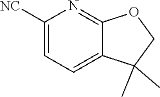

To a solution of 3,3-dimethyl-2,3-dihydrofuro[2,3-b]pyridine 7-oxide (1.0 g, 6.05 mmol) in acetonitrile (10 mL) was added TMSCN (5.28 g, 53.3 mmol) and triethylamine (1.96 g, 19.4 mmol). The mixture was stirred at 100° C. for 1 h. The mixture was cooled to room temperature and concentrated under vacuum. The residue was purified by silica gel chromatograph (PE/EtOAc=1/0 to 1/1) to give the title compound (0.80 g, 76%). ¹H NMR (400 MHz, DMSO-d6) δ 7.88 (d, J=7.2 Hz, 1H), 4.36 (s, 2H), 1.34 (s, 6H).

### Step 5: (3,3-dimethyl-2,3-dihydrofuro[2,3-b]pyridin-6-yl)methanamine

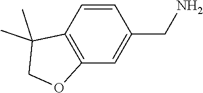

To a mixture of 3,3-dimethyl-2,3-dihydrofuro[2,3-b]pyridine-6-carbonitrile (0.40 g, 2.30 mmol), (Boc)₂O (1.0 g, 4.59 mmol) and NiCl₂ (89 mg, 0.69 mmol) in MeOH (6 mL) was added NaBH₄ (347 mg, 9.18 mmol) portion-wise at 0° C. The mixture was warmed to 25° C. and stirred for 2 h. Saturated NH₄Cl (1 mL) was added into the reaction mixture dropwise. The mixture was concentrated under reduced pressure after cease of gas evolution. The residue was triturated in EtOAc and filtered. The filtrate was concentrated under reduced pressure to give crude tert-butyl ((3,3-dimethyl-2,3-dihydrofuro[2,3-b]pyridin-6-yl)methyl)carbamate (0.70 g). To the crude was added methanolic HCl (4 M, 1 mL) and the mixture was stirred at 25° C. for 0.5 h. The mixture was concentrated in vacuum and the residue was redissolved in MeOH (1 mL). The mixture was neutralized with methanolic NaOH (1 M) to pH 7. The mixture was triturated in DCM (10 mL). The filtrate was collected by filtration and concentrated under vacuum to give the title compound (160 mg, 39%). LC-MS (M+H)⁺=179.3.

### Step 6: N-((3,3-dimethyl-2,3-dihydrofuro[2,3-b]pyridin-6-yl)methyl)-5,6,7,8-tetrahydroquinolin-8-amine trifluoroacetate

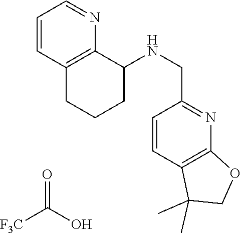 [SMILES: O=C(O)C(F)(F)F ; CC1(C)COc2nc(CNC3CCCc4cccnc43)ccc21]

To a solution of (3,3-dimethyl-2,3-dihydrofuro[2,3-b]pyridin-6-yl)methanamine (0.15 g, 0.84 mmol) and 6,7-dihydroquinolin-8(5H)-one (83 mg, 0.56 mmol) in DCM (2 mL) and MeOH (0.2 mL) was added NaBH(OAc)₃ (238 mg, 1.12 mmol) at 25° C. After 30 min, saturated NaHCO₃ (5 mL) was added, and the mixture was extracted with DCM (3 mL×5). The combined organic layer was washed with brine (3 mL), dried over anhydrous Na₂SO₄, filtered and concentrated under vacuum. The residue was purified by prep-HPLC to give the title compound (105 mg, 60%). LC-MS (M+H)⁺=310.0.

### Step 7: tert-butyl ((7-(1-(((3,3-dimethyl-2,3-dihydrofuro[2,3-b]pyridin-6-yl)methyl)(5,6,7,8-tetrahydroquinolin-8-yl)carbamoyl)cyclopropyl)-4-oxo-3,4-dihydrophthalazin-1-yl)methyl)carbamate

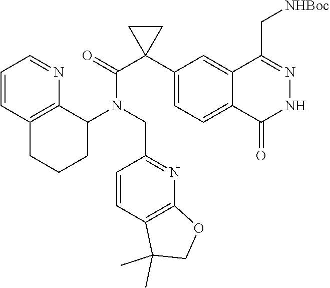 [SMILES: CC(C)(C)OC(=O)NCc1n[nH]c(=O)c2ccc(C3(C(=O)N(Cc4ccc5c(n4)OCC5(C)C)C4CCCc5cccnc54)CC3)cc12]

To a solution of 1-(4-(((tert-butoxycarbonyl)amino)methyl)-1-oxo-1,2-dihydrophthalazin-6-yl)cyclopropane-1-carboxylic acid (30 mg, 0.08 mmol) in DMF (2.5 mL) was added HATU (35 mg, 0.09 mmol), N-((3,3-dimethyl-2,3-dihydrofuro[2,3-b]pyridin-6-yl)methyl)-5,6,7,8-tetrahydroquinolin-8-amine trifluoroacetate (28 mg, 0.065 mmol) and DIPEA (21 mg, 0.17 mmol). The mixture was stirred for 3 h at 40° C. The reaction mixture was cooled to room temperature and diluted with water (100 mL). The mixture was extracted with EtOAc (100 mL). The combined organic layer was washed with brine (100 mL×2), dried over Na₂SO₄, filtered and concentrated under vacuum. The residue was purified by silica gel chromatograph (DCM:MeOH=20:1) to give the title compound (20 mg, 47%). LC-MS (M+H)⁺=651.2.

### Step 8: 1-(4-(aminomethyl)-1-oxo-1,2-dihydrophthalazin-6-yl)-N-((3,3-dimethyl-2,3-dihydrofuro[2,3-b]pyridin-6-yl)methyl)-N-(5,6,7,8-tetrahydroquinolin-8-yl)cyclopropane-1-carboxamide

To a solution of tert-butyl ((7-(1-(((3,3-dimethyl-2,3-dihydrofuro[2,3-b]pyridin-6-yl)methyl)(5,6,7,8-tetrahydroquinolin-8-yl)carbamoyl)cyclopropyl)-4-oxo-3,4-dihydrophthalazin-1-yl)methyl)carbamate (20 mg, 0.03 mmol) in DCM (4 mL) was added TFA (1 mL). The mixture was stirred for 1 h at room temperature then concentrated under vacuum. The residue was purified by prep-HPLC to give Example 5 (10 mg, 60%); H NMR (500 MHz, DMSO-d6) δ 12.65-12.20 (m, 1H), 8.49-8.36 (m, 1H), 8.32-7.63 (m, 3H), 7.60-7.09 (m, 3H), 6.76-6.61 (m, 1H), 5.62-3.88 (m, 6H), 3.30-3.20 (m, 1H), 2.83-2.57 (m, 2H), 2.31-1.79 (m, 3H), 1.78-1.34 (m, 5H), 1.35-1.05 (m, 7H), 0.93-0.75 (m, 1H). LC-MS (M+H)⁺=551.4.

### Example 6: 1-(4-(aminomethyl)-1-oxo-1,2-dihydrophthalazin-6-yl)-N-((5-(2-hydroxypropan-2-yl)pyridin-2-yl)methyl)-N-(5,6,7,8-tetrahydroquinolin-8-yl)cyclopropane-1-carboxamide

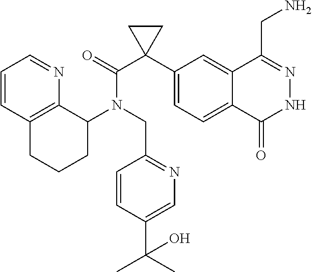 [SMILES: CC(C)(O)c1ccc(CN(C(=O)C2(c3ccc4c(=O)[nH]nc(CN)c4c3)CC2)C2CCCc3cccnc32)nc1]

### Step 1: methyl 6-(((tert-butoxycarbonyl)amino)methyl)nicotinate

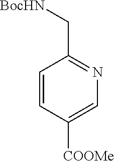 [SMILES: COC(=O)c1ccc(CNC(=O)OC(C)(C)C)nc1]

To a solution of methyl 6-cyanonicotinate (5.0 g, 30.8 mmol) in MeOH (100 mL) was added Boc₂O (13.5 g, 61.7 mmol) and Pd/C (10%, 0.50 g) at 25° C. under nitrogen. The mixture was purged with hydrogen for three times and stirred at 25° C. at 15 psi of hydrogen for 12 h. The mixture was filtered and the residue was rinsed with EtOAc (30 mL). Water (30 mL) was added to the filtrate and the mixture was extracted with EtOAc (30 mL×3). The combined organic layer was washed with brine (10 mL×3), dried over Na₂SO₄, filtered and concentrated under vacuum. The residue was purified by silica gel chromatograph (PE/EtOAc=3/1) to give the title compound (7.0 g, 85%). LC-MS (M+H)⁺=267.1.

### Step 2: tert-butyl ((5-(2-hydroxypropan-2-yl)pyridin-2-yl)methyl)carbamate

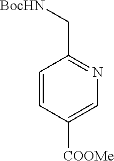 [SMILES: COC(=O)c1ccc(CNC(=O)OC(C)(C)C)nc1]

To a solution of MeMgBr in THF (3.0 M, 1.88 mL, 5.6 mmol) in THF (6 mL) was slowly added a solution of methyl 6-(((tert-butoxycarbonyl)amino)methyl)nicotinate (0.50 g, 1.88 mmol) in THF (4 mL) at 0° C. under nitrogen. The mixture was stirred at 25° C. for 12 h and aqueous HCl (0.5 M, 10 mL) was added carefully. Organic layer was separated and the aqueous layer was extracted with ethyl acetate (5 mL×3). The combined organic layer was washed with brine (10 mL), dried over anhydrous Na₂SO₄, filtered and concentrated under vacuum. The residue was purified by silica gel chromatograph (PE/EtOAc=1/0 to 1/1) to give the title compound (0.30 g, 60%). ¹H NMR (400 MHz, DMSO-d6) δ 8.58-8.55 (m, 1H), 7.79 (dd, J=8.0, 2.4 Hz, 1H), 7.17 (d, J=8.0 Hz 1H), 5.16 (s, 1H), 4.19 (d, J=6.0 Hz 1H), 1.43 (s, 6H), 1.40 (s, 9H).

### Step 3: 2-(6-(((5,6,7,8-tetrahydroquinolin-8-yl)amino)methyl)pyridin-3-yl)propan-2-ol

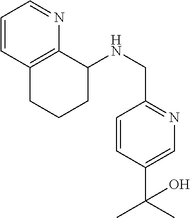 [SMILES: CC(C)(O)c1ccc(CNC2CCCc3cccnc32)nc1]

A solution of tert-butyl ((5-(2-hydroxypropan-2-yl)pyridin-2-yl)methyl)carbamate (0.30 g, 1.13 mmol) in methanolic HCl (4 M, 0.5 mL) was stirred at 25° C. for 0.5 h, and the mixture was concentrated under reduced pressure. The residue was redissolved in DCM (2 mL) and MeOH (0.2 mL) followed by addition of 6,7-dihydroquinolin-8(5H)-one (89 mg, 0.60 mmol) and NaBH(OAc)₃ (255 mg, 1.20 mmol) at 25° C. After 0.5 h, the mixture was carefully quenched with saturated NaHCO₃ (5 mL) and extracted with DCM (3 mL×5). The combined organic layer was washed with brine (3 mL), dried with Na₂SO₄, filtered, and concentrated under vacuum. The residue was purified by prep-HPLC to give the title compound (72 mg, 40%). LC-MS (M+H)⁺=298.2.

### Step 4: tert-butyl ((7-(1-(((5-(2-hydroxypropan-2-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

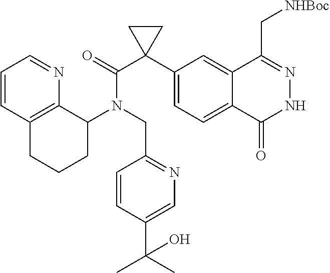 [SMILES: CC(C)(C)OC(=O)NCc1n[nH]c(=O)c2ccc(C3(C(=O)N(Cc4ccc(C(C)(C)O)cn4)C4CCCc5cccnc54)CC3)cc12]

To a solution of 1-(4-(((tert-butoxycarbonyl)amino)methyl)-1-oxo-1,2-dihydrophthalazin-6-yl)cyclopropane-1-carboxylic acid (30 mg, 0.08 mmol) in DMF (2.5 mL) was added HATU (35 mg, 0.09 mmol), 2-(6-(((5,6,7,8-tetrahydroquinolin-8-yl)amino)methyl)pyridin-3-yl)propan-2-ol (27.3 mg, 0.09 mmol) and DIPEA (21.5 mg, 0.17 mmol). The mixture was stirred for 3 h at 40° C. The mixture was cooled to room temperature, diluted with water (100 mL) and then extracted with EtOAc (100 mL). The organic layer was separated, washed with brine (100 mL×2), dried over Na₂SO₄, filtered and concentrated under vacuum. The residue was purified by silica gel chromatograph (DCM:MeOH=20:1) to give the title compound (20 mg, 56%). LC-MS (M+H)⁺=639.2.

### Step 5: 1-(4-(aminomethyl)-1-oxo-1,2-dihydrophthalazin-6-yl)-N-((5-(2-hydroxypropan-2-yl)pyridin-2-yl)methyl)-N-(5,6,7,8-tetrahydroquinolin-8-yl)cyclopropane-1-carboxamide

To a solution of tert-butyl ((7-(1-(((5-(2-hydroxypropan-2-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate (20 mg, 0.03 mmol) in DCM (3 mL) was added TFA (1 mL). The mixture was stirred for 1 h at room temperature then concentrated under vacuum. The residue was purified by prep-HPLC to give Example 6 (9 mg, 53%); ¹H NMR (500 MHz, DMSO-d6) δ 12.77-12.16 (m, 1H), 8.65-8.19 (m, 3H), 8.14-7.35 (m, 4H), 7.27-7.01 (m, 2H), 5.64-4.32 (m, 3H), 4.06-3.79 (m, 2H), 3.59-3.37 (m, 1H), 2.85-2.56 (m, 2H), 2.29-1.01 (m, 15H), 0.99-0.79 (m, 1H). LC-MS (M+H)⁺=539.5.

### Example 7: 1-(4-(aminomethyl)-1-oxo-1,2-dihydrophthalazin-6-yl)-N-((4-methyl-5-(trifluoromethyl)pyridin-2-yl)methyl)-N-(5,6,7,8-tetrahydroquinolin-8-yl)cyclopropane-1-carboxamide

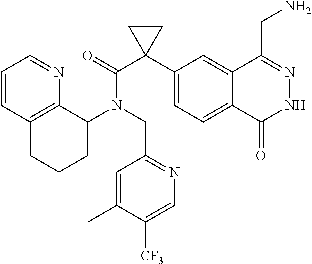 [SMILES: Cc1cc(CN(C(=O)C2(c3ccc4c(=O)[nH]nc(CN)c4c3)CC2)C2CCCc3cccnc32)ncc1C(F)(F)F]

### Step 1: 4-methyl-5-(trifluoromethyl)picolinonitrile

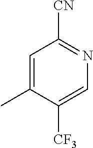

To a solution of 2-chloro-4-methyl-5-(trifluoromethyl)pyridine (500 mg, 2.56 mmol) in DMF (5 mL) was added Zn(CN)₂ (1.20 g, 10.23 mmol) and Pd(PPh₃)₄(295 mg, 0.26 mmol). The mixture was degassed and purged with nitrogen for 3 times then warmed to 80° C. After 3 h, the mixture was cooled to room temperature, diluted with water (20 mL) and extracted with EtOAc (10 mL×3). The combined organic layer was washed with brine (20 mL), dried over Na₂SO₄, filtered and concentrated under reduced pressure. The residue was purified by silica gel chromatograph (PE:EtOAc=1:0 to 1:1) to give the title compound (200 mg, 42%) LC-MS (M+H)⁺=187.2.

### Step 2: tert-butyl ((4-methyl-5-(trifluoromethyl)pyridin-2-yl)methyl)carbamate

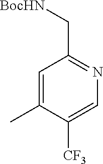 [SMILES: Cc1cc(CNC(=O)OC(C)(C)C)ncc1C(F)(F)F]

To a mixture of 4-methyl-5-(trifluoromethyl)picolinonitrile (300 mg, 1.61 mmol), NiCl₂ (62 mg, 0.48 mmol) and Boc₂O (704 mg, 3.22 mmol) in MeOH (4.5 mL) was added NaBH₄ (122 mg, 3.22 mmol) at 0° C. The mixture was warmed to 25° C. and stirred for 2 h. Water (10 mL) was added and the mixture was extracted with EtOAc (5 mL×3). The combined organic layers were washed with brine (10 mL), dried over Na₂SO₄, filtered and concentrated under reduced pressure. The residue was purified by silica gel chromatograph (PE:EtOAc=10:1 to 1:1) to give the title compound (300 mg, 1.03 mmol, 64% yield). ¹H NMR (400 MHz, CDCl₃) δ 8.71 (s, 1H), 7.22 (s, 1H), 5.53 (br s, 1H), 4.46 (d, J=5.6 Hz, 2H), 2.50 (s, 3H), 1.47 (s, 9H).

### Step 3: N-((4-methyl-5-(trifluoromethyl)pyridin-2-yl)methyl)-5,6,7,8-tetrahydroquinolin-8-amine

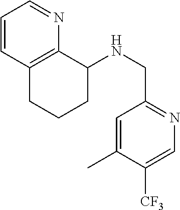 [SMILES: Cc1cc(CNC2CCCc3cccnc32)ncc1C(F)(F)F]

The title compound (130 mg, 37%) was prepared in a manner similar to that in Example 6 step 3 from tert-butyl ((4-methyl-5-(trifluoromethyl)pyridin-2-yl)methyl)carbamate and 6,7-dihydroquinolin-8(5H)-one. LCMS (M+H)⁺=322.1.

### Step 4: tert-butyl ((7-(1-(((4-methyl-5-(trifluoromethyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

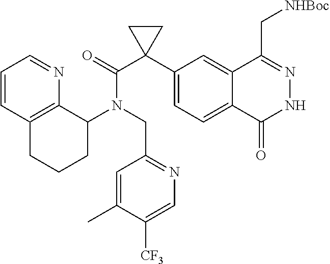 [SMILES: Cc1cc(CN(C(=O)C2(c3ccc4c(=O)[nH]nc(CNC(=O)OC(C)(C)C)c4c3)CC2)C2CCCc3cccnc32)ncc1C(F)(F)F]

The title compound (30 mg, 41%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((4-methyl-5-(trifluoromethyl)pyridin-2-yl)methyl)-5,6,7,8-tetrahydroquinolin-8-amine. LCMS (M+H)⁺=663.4.

### Step 5: 1-(4-(aminomethyl)-1-oxo-1,2-dihydrophthalazin-6-yl)-N-((4-methyl-5-(trifluoromethyl)pyridin-2-yl)methyl)-N-(5,6,7,8-tetrahydroquinolin-8-yl)cyclopropane-1-carboxamide

Example 7 (10 mg, 39%) was prepared in a manner similar to that in Example 6 step 5 from tert-butyl ((7-(1-(((4-methyl-5-(trifluoromethyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.54-12.34 (m, 1H), 8.78-8.44 (m, 1H), 8.43-8.39 (m, 1H), 8.31-8.27 (m, 1H), 8.08-7.96 (m, 1H), 7.94-7.64 (m, 1H), 7.61-7.20 (m, 2H), 7.20-7.12 (m, 1H), 5.61-4.97 (m, 1H), 4.70-4.61 (m, 1H), 4.09-3.85 (m, 2H), 3.63-3.54 (m 1H), 2.71-2.58 (m, 2H), 2.42-2.01 (m, 3H), 1.94-1.84 (m, 1H), 1.72-1.23 (m, 6H), 1.00-0.83 (m, 1H). LCMS (M+H)⁺=563.4.

### Example 8: 1-(4-(aminomethyl)-1-oxo-1,2-dihydrophthalazin-6-yl)-N-((6-methyl-5-phenylpyridin-2-yl)methyl)-N-(5,6,7,8-tetrahydroquinolin-8-yl)cyclopropane-1-carboxamide

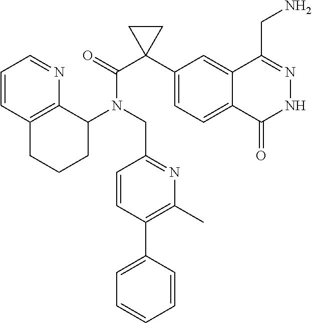 [SMILES: Cc1nc(CN(C(=O)C2(c3ccc4c(=O)[nH]nc(CN)c4c3)CC2)C2CCCc3cccnc32)ccc1-c1ccccc1]

### Step 1: tert-butyl ((6-methyl-5-phenylpyridin-2-yl)methyl)carbamate

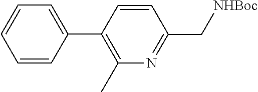 [SMILES: Cc1nc(CNC(=O)OC(C)(C)C)ccc1-c1ccccc1]

The title compound 0.60 g, 49%) was prepared in a manner similar to that in Example 7 step 2 from 6-methyl-5-phenylpicolinonitrile. LCMS (M+H)⁺=299.2.

### Step 2: N-((6-methyl-5-phenylpyridin-2-yl)methyl)-5,6,7,8-tetrahydroquinolin-8-amine

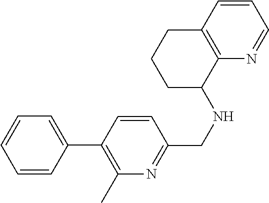 [SMILES: Cc1nc(CNC2CCCc3cccnc32)ccc1-c1ccccc1]

The title compound (65 mg, 39%) was prepared in a manner similar to that in Example 6 step 3 from tert-butyl ((6-methyl-5-phenylpyridin-2-yl)methyl)carbamate and 6,7-dihydroquinolin-8(5H)-one. LCMS (M+H)⁺=330.2.

### Step 3: tert-butyl ((7-(1-(((6-methyl-5-phenylpyridin-2-yl)methyl)(5,6,7,8-tetrahydroquinolin-8-yl)carbamoyl)cyclopropyl)-4-oxo-3,4-dihydrophthalazin-1-yl)methyl)carbamate

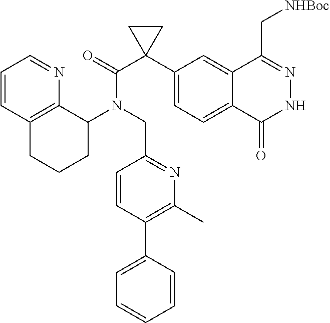 [SMILES: Cc1nc(CN(C(=O)C2(c3ccc4c(=O)[nH]nc(CNC(=O)OC(C)(C)C)c4c3)CC2)C2CCCc3cccnc32)ccc1-c1ccccc1]

The title compound (20 mg, 35%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((6-methyl-5-phenylpyridin-2-yl)methyl)-5,6,7,8-tetrahydroquinolin-8-amine. LC-MS (M+H)⁺=671.4.

### Step 4: 1-(4-(aminomethyl)-1-oxo-1,2-dihydrophthalazin-6-yl)-N-((6-methyl-5-phenylpyridin-2-yl)methyl)-N-(5,6,7,8-tetrahydroquinolin-8-yl)cyclopropane-1-carboxamide

Example 8 (8 mg, 47%) was prepared in a manner similar to that in Example 6 step 5 from tert-butyl ((7-(1-(((6-methyl-5-phenylpyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (400 MHz, DMSO-d6) δ 12.6-12.2 (s, 1H), 8.48-8.41 (m, 1H), 8.29-8.25 (m, 2H), 8.10-8.06 (m, 1H), 7.89-7.68 (m, 1H), 7.56-7.38 (m, 6H), 7.23-7.06 (m, 3H), 5.56-5.61 (m, 1H), 4.86-4.70 (m, 1H), 4.18-4.10 (m, 2H), 3.52-3.48 (m, 2H), 2.78-2.56 (m, 2H), 2.4-2.37 (m, 3H), 2.23-2.13 (m, 1H), 2.02-1.88 (m, 1H), 1.72-1.27 (m, 6H), 0.94-0.85 (m, 1H). LC-MS (M+H)⁺=571.4.

### Example 9: 1-(4-(aminomethyl)-1-oxo-1,2-dihydrophthalazin-6-yl)-N-(2-chloro-6-fluorobenzyl)-N-((5-(trifluoromethyl)pyridin-2-yl)methyl)cyclopropane-1-carboxamide

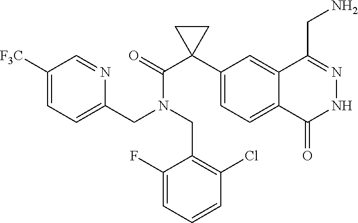 [SMILES: NCc1n[nH]c(=O)c2ccc(C3(C(=O)N(Cc4ccc(C(F)(F)F)cn4)Cc4c(F)cccc4Cl)CC3)cc12]

### Step 1: N-(2-chloro-6-fluorobenzyl)-1-(5-(trifluoromethyl)pyridin-2-yl)methanamine

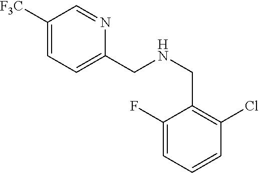 [SMILES: Fc1cccc(Cl)c1CNCc1ccc(C(F)(F)F)cn1]

The title compound (90 mg, 43%) was prepared in a manner similar to that in Example 3 step 3 from (5-(trifluoromethyl)pyridin-2-yl)methanamine and 2-chloro-6-fluorobenzaldehyde. LC-MS (M+H)⁺=319.1.

### Step 2: tert-butyl ((7-(1-((2-chloro-6-fluorobenzyl)((5-(trifluoromethyl)pyridin-2-yl)methyl)carbamoyl)cyclopropyl)-4-oxo-3,4-dihydrophthalazin-1-yl)methyl)carbamate

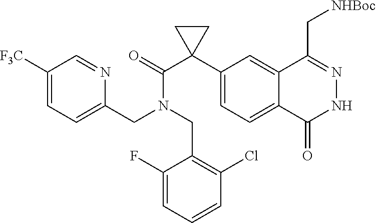 [SMILES: CC(C)(C)OC(=O)NCc1n[nH]c(=O)c2ccc(C3(C(=O)N(Cc4ccc(C(F)(F)F)cn4)Cc4c(F)cccc4Cl)CC3)cc12]

The title compound (15 mg, 23%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-(2-chloro-6-fluorobenzyl)-1-(5-(trifluoromethyl)pyridin-2-yl)methanamine. LC-MS (M+H)⁺=660.4.

### Step 3: 1-(4-(aminomethyl)-1-oxo-1,2-dihydrophthalazin-6-yl)-N-(2-chloro-6-fluorobenzyl)-N-((5-(trifluoromethyl)pyridin-2-yl)methyl)cyclopropane-1-carboxamide

Example 9 (6 mg, 47%) was prepared in a manner similar to that in Example 6 step 5 from tert-butyl ((7-(1-((2-chloro-6-fluorobenzyl)((5-(trifluoromethyl)pyridin-2-yl)methyl)carbamoyl)cyclopropyl)-4-oxo-3,4-dihydrophthalazin-1-yl)methyl)carbamate. ¹H NMR (400 MHz, DMSO-d6) δ 12.41 (s, 1H), 8.74 (s, 1H), 8.25-7.01 (m, 8H), 4.84-4.57 (m, 4H), 4.03-3.90 (m, 2H), 1.66-1.24 (m, 4H). LC-MS (M+H)⁺=560.3.

### Example 10: 1-(4-(aminomethyl)-1-oxo-1,2-dihydrophthalazin-6-yl)-N-((5-phenylpyridin-2-yl)methyl)-N-(5,6,7,8-tetrahydroquinolin-8-yl)cyclopropane-1-carboxamide

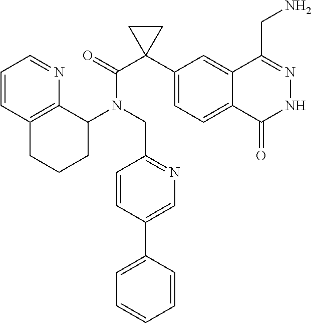 [SMILES: NCc1n[nH]c(=O)c2ccc(C3(C(=O)N(Cc4ccc(-c5ccccc5)cn4)C4CCCc5cccnc54)CC3)cc12]

### Step 1: tert-butyl ((5-phenylpyridin-2-yl)methyl)carbamate

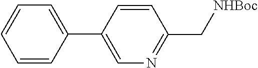 [SMILES: CC(C)(C)OC(=O)NCc1ccc(-c2ccccc2)cn1]

The title compound (170 mg, 86%) was prepared in a manner similar to that in Example 3 step 1 from tert-butyl ((5-bromopyridin-2-yl)methyl)carbamate and phenylboronic acid. LC-MS (M+H)⁺=285.1.

### Step 2: N-((5-phenylpyridin-2-yl)methyl)-5,6,7,8-tetrahydroquinolin-8-amine

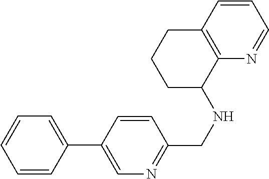 [SMILES: c1ccc(-c2ccc(CNC3CCCc4cccnc43)nc2)cc1]

The title compound (188 mg, 78%) was prepared in a manner similar to that in Example 6 step 3 from tert-butyl ((5-phenylpyridin-2-yl)methyl)carbamate and 6,7-dihydroquinolin-8(5H)-one. LCMS (M+H)⁺=316.2.

### Step 3: tert-butyl ((4-oxo-7-(1-(((5-phenylpyridin-2-yl)methyl)(5,6,7,8-tetrahydroquinolin-8-yl)carbamoyl)cyclopropyl)-3,4-dihydrophthalazin-1-yl)methyl)carbamate

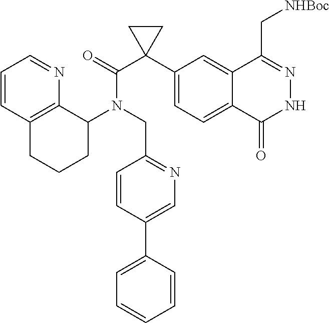 [SMILES: CC(C)(C)OC(=O)NCc1n[nH]c(=O)c2ccc(C3(C(=O)N(Cc4ccc(-c5ccccc5)cn4)C4CCCc5cccnc54)CC3)cc12]

The title compound (30 mg, 46%) was prepared in a manner similar to that in Example 6 step 4 from N-((5-phenylpyridin-2-yl)methyl)-5,6,7,8-tetrahydroquinolin-8-amine and 1-(4-(((tert-butoxycarbonyl)amino)methyl)-1-oxo-1,2-dihydrophthalazin-6-yl)cyclopropane-1-carboxylic acid. LC-MS (M+H)⁺=657.5.

### Step 4: 1-(4-(aminomethyl)-1-oxo-1,2-dihydrophthalazin-6-yl)-N-((5-phenylpyridin-2-yl)methyl)-N-(5,6,7,8-tetrahydroquinolin-8-yl)cyclopropane-1-carboxamide

Example 10 (15 mg, 59%) was prepared in a manner similar to that in Example 6 step 5 from tert-butyl ((4-oxo-7-(1-(((5-phenylpyridin-2-yl)methyl)(5,6,7,8-tetrahydroquinolin-8-yl)carbamoyl)cyclopropyl)-3,4-dihydrophthalazin-1-yl)methyl)carbamate. ¹H NMR (400 MHz, DMSO-d6) δ 12.55-12.32 (m, 1H), 8.81-8.59 (m, 1H), 8.47-8.41 (m, 1H), 8.35-7.36 (m, 11H), 7.24-7.17 (m, 1H), 5.58-4.74 (m, 2H), 4.09-3.99 (m, 2H), 3.55-3.52 (m, 1H), 2.75-2.58 (m, 2H), 2.32-0.88 (m, 10H). LC-MS (M+H)⁺=557.4.

### Example 11A & 11B: (R)-1-(4-(aminomethyl)-1-oxo-1,2-dihydrophthalazin-6-yl)-N-((5-phenylpyridin-2-yl)methyl)-N-(5,6,7,8-tetrahydroquinolin-8-yl)cyclopropane-1-carboxamide & (S)-1-(4-(aminomethyl)-1-oxo-1,2-dihydrophthalazin-6-yl)-N-((5-phenylpyridin-2-yl)methyl)-N-(5,6,7,8-tetrahydroquinolin-8-yl)cyclopropane-1-carboxamide

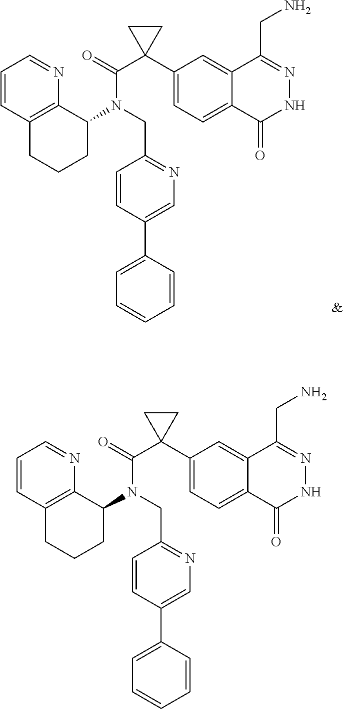 [SMILES: NCc1n[nH]c(=O)c2ccc(C3(C(=O)N(Cc4ccc(-c5ccccc5)cn4)[C@@H]4CCCc5cccnc54)CC3)cc12 ; NCc1n[nH]c(=O)c2ccc(C3(C(=O)N(Cc4ccc(-c5ccccc5)cn4)[C@H]4CCCc5cccnc54)CC3)cc12]

Example 10 (38 mg) was separated by chiral prep-HPLC to give Example 11A (7 mg, 18%) and Example 11B (9 mg, 24%). Analytical chiral-HPLC condition as below. Column: CHIRALPAK IC-3; Column size: 4.6×50 mm, 3 m; Mobile phase: (Hexane:DCM=1:1, with 0.1% diethylamine):EtOH=50:50; Flow: 1 mL/min; Temperature: 25° C.

Example 11A: Chiral HPLC t_{R}=2.71 min. ¹H NMR (400 MHz, DMSO-d6) δ 12.55-12.30 (s, 1H), 8.83-8.55 (m, 1H), 8.47-8.41 (m, 1H), 8.35-7.36 (m, 11H), 7.24-7.17 (m, 1H), 5.58-4.74 (m, 2H), 4.09-3.99 (m, 2H), 3.55-3.52 (m, 1H), 2.75-2.58 (m, 2H), 2.32-0.88 (m, 10H). LC-MS (M+H)⁺=557.4.

Example 11B: Chiral HPLC t_{R}=5.90 min. ¹H NMR (400 MHz, DMSO-d6) δ 12.55-12.30 (s, 1H), 8.83-8.55 (m, 1H), 8.47-8.41 (m, 1H), 8.35-7.36 (m, 11H), 7.24-7.17 (m, 1H), 5.58-4.74 (m, 2H), 4.09-3.99 (m, 2H), 3.55-3.52 (m, 1H), 2.75-2.58 (m, 2H), 2.32-0.88 (m, 10H). LC-MS (M+H)⁺=557.4.

### Example 12: 1-(4-(aminomethyl)-1-oxo-1,2-dihydrophthalazin-6-yl)-N-((5-iodo-4-methylthiazol-2-yl)methyl)-N-(5,6,7,8-tetrahydroquinolin-8-yl)cyclopropane-1-carboxamide

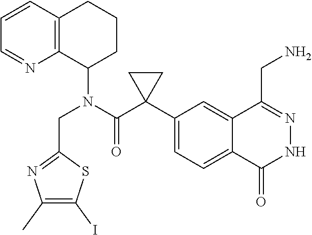 [SMILES: Cc1nc(CN(C(=O)C2(c3ccc4c(=O)[nH]nc(CN)c4c3)CC2)C2CCCc3cccnc32)sc1I]

### Step 1: 2-(1,3-dioxolan-2-yl)-4-methylthiazole

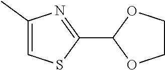

To a solution of 4-methylthiazole-2-carbaldehyde (1.0 g, 7.86 mmol) in toluene (10 mL) was added ethylene glycol (1.46 g, 23.6 mmol) and TsOH monohydrate (449 mg, 2.36 mmol) and the mixture was stirred at 110° C. for 12 h. The mixture was cooled to room temperature and concentrated under reduced pressure. The residue was purified by silica gel chromatograph (PE:EtOAc=1:0 to 5:1) to give the title compound (1.0 g, 74%). ¹H NMR (400 MHz, CDCl₃) δ 6.92 (s, 1H), 6.12 (s, 1H), 4.20-4.03 (m, 4H), 2.47 (s, 3H).

### Step 2: 2-(1,3-dioxolan-2-yl)-5-iodo-4-methylthiazole

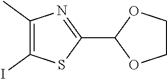

To a solution of 2-(1,3-dioxolan-2-yl)-4-methylthiazole (1.0 g, 5.84 mmol) in anhydrous THF (30 mL) was added n-BuLi (2.5 M, 2.34 mL, 4.58 mmol) at −78° C. dropwise, followed by I₂ (1.48 g, 5.84 mmol). The mixture was stirred at −78° C. for 2 h, then quenched with saturated NH₄Cl (30 mL), warmed to room temperature and then extracted with EtOAc (20 mL×3). The combined organic layer was washed with brine (10 mL×2), dried over Na₂SO₄, filtered and concentrated under reduced pressure. The residue was purified by silica gel chromatograph (PE/EtOAc=1/0 to 1/1) to give the title compound (1.0 g, 58%). ¹H NMR (400 MHz, CDCl₃) δ 6.09 (s, 1H), 4.15-4.05 (m, 4H), 2.47 (s, 3H).

### Step 3: 5-iodo-4-methylthiazole-2-carbaldehyde

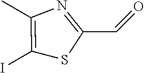

To a solution of 2-(1,3-dioxolan-2-yl)-5-iodo-4-methylthiazole (1.0 g, 3.37 mmol) in acetone (10 mL) and water (4 mL) was added concentrated HCl (4.2 mL) and the mixture was stirred at 60° C. for 3 h. The mixture was cooled to room temperature and neutralized with saturated NaHCO₃ solution until its pH became 7. The mixture was extracted with DCM (5 mL×5). The combined organic layer was washed with brine (10 mL) and concentrated under vacuum. The residue was purified by silica gel chromatograph (PE/EtOAc=1/0 to 10/1) to give the title compound (0.50 g, 59%). ¹H NMR (400 MHz, CDCl₃) δ 9.85 (s, 1H), 2.58 (s, 3H).

### Step 4: N-((5-iodo-4-methylthiazol-2-yl)methyl)-5,6,7,8-tetrahydroquinolin-8-amine

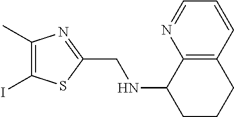 [SMILES: Cc1nc(CNC2CCCc3cccnc32)sc1I]

To a solution of 5,6,7,8-tetrahydroquinolin-8-amine (150 mg, 1.01 mmol) and 5-iodo-4-methylthiazole-2-carbaldehyde (171 mg, 0.67 mmol) in DCM (2 mL) and MeOH (0.2 mL) was added NaBH(OAc)₃ (286 mg, 1.35 mmol) at 25° C. The mixture was stirred at 25° C. for 0.5 h then quenched with aq. NaHCO₃ (5 mL). The mixture was extracted with DCM (3 mL×5). The combined organic layer was washed with brine (3 mL), dried over Na₂SO₄, filtered and concentrated under vacuum. The residue was purified by prep-HPLC to give the title compound (31 mg, 12%). LC-MS (M+H)⁺=386.0.

### Step 5: tert-butyl ((7-(1-(((5-iodo-4-methylthiazol-2-yl)methyl)(5,6,7,8-tetrahydroquinolin-8-yl)carbamoyl)cyclopropyl)-4-oxo-3,4-dihydrophthalazin-1-yl)methyl)carbamate

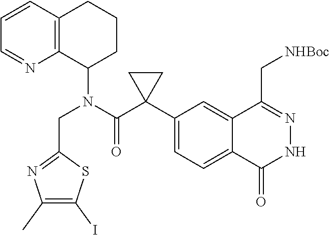 [SMILES: Cc1nc(CN(C(=O)C2(c3ccc4c(=O)[nH]nc(CNC(=O)OC(C)(C)C)c4c3)CC2)C2CCCc3cccnc32)sc1I]

The title compound (30 mg, 51%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-iodo-4-methylthiazol-2-yl)methyl)-5,6,7,8-tetrahydroquinolin-8-amine. LC-MS (M+H)⁺=727.1.

### Step 6: 1-(4-(aminomethyl)-1-oxo-1,2-dihydrophthalazin-6-yl)-N-((5-iodo-4-methylthiazol-2-yl)methyl)-N-(5,6,7,8-tetrahydroquinolin-8-yl)cyclopropane-1-carboxamide

Example 12 (8 mg, 32%) was prepared in a manner similar to that in Example 6 step 5 from tert-butyl ((7-(1-(((5-iodo-4-methylthiazol-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.60 (s, 1H), 8.35 (d, J=4.4 Hz, 1H), 8.21 (q, J=5.8 Hz, 2H), 7.89-7.82 (m, 2H), 7.51 (d, J=7.5 Hz, 1H), 7.21 (dd, J=7.6, 4.7 Hz, 1H), 5.51-5.48 (m, 1H), 4.74-4.71 (m, 1H), 4.17-4.01 (m, 2H), 3.72-3.69 (m, 1H), 2.72-2.60 (m, 2H), 2.28 (s, 3H), 1.92-1.83 (m, 1H), 1.74-1.54 (m, 4H), 1.50-1.34 (m, 2H), 1.10 (t, J=7.1 Hz, 1H), 0.97-0.94 (m, 1H). LC-MS (M+H)⁺=627.1.

### Example 13: 1-(4-(aminomethyl)-1-oxo-1,2-dihydrophthalazin-6-yl)-N-((5-(2-chloro-6-cyanophenyl)pyridin-2-yl)methyl)-N-(5,6,7,8-tetrahydroquinolin-8-yl)cyclopropane-1-carboxamide

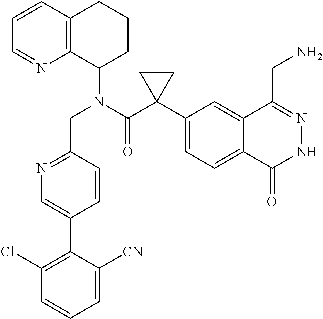 [SMILES: N#Cc1cccc(Cl)c1-c1ccc(CN(C(=O)C2(c3ccc4c(=O)[nH]nc(CN)c4c3)CC2)C2CCCc3cccnc32)nc1]

### Step 1: tert-butyl ((5-(2-chloro-6-cyanophenyl)pyridin-2-yl)methyl)carbamate

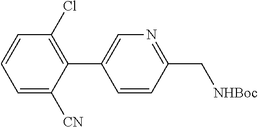 [SMILES: CC(C)(C)OC(=O)NCc1ccc(-c2c(Cl)cccc2C#N)cn1]

To a solution of tert-butyl ((5-bromopyridin-2-yl)methyl)carbamate (1.0 g, 3.48 mmol) in dioxane (10 mL) was BPD (884 mg, 3.48 mmol), KOAc (854 mg, 8.71 mmol) and Pd(dppf)Cl₂ (255 mg, 0.35 mmol) under nitrogen. The mixture was stirred at 85° C. for 16 h, cooled to room temperature and filtered. The filtrate was concentrated to give a crude (2.0 g). A quarter of the crude (0.50 g), 3-chloro-2-iodobenzonitrile (418 mg, 1.59 mmol) was dissolved in dioxane (2.5 mL), toluene (1.3 mL) and water (1.3 mL), followed by addition of Pd(dppf)Cl₂ (145 mg, 0.20 mmol) and K₃PO₄ (1.05 g, 4.96 mmol). The mixture was stirred at 85° C. for 3 h under nitrogen. The mixture was cooled to room temperature, diluted with water (10 mL) and extracted with EtOAc (8.0 mL×2). The combined organic layer was washed with brine (5.0 mL), dried with Na₂SO₄, filtered and concentrated under vacuum. The residue was purified by silica gel chromatograph (PE:EtOAc=50:1 to 5:1) to give the title compound (240 mg, 80%). LC-MS (M+H)⁺=344.1.

### Step 2: 3-chloro-2-(6-(((5,6,7,8-tetrahydroquinolin-8-yl)amino)methyl)pyridin-3-yl)benzonitrile

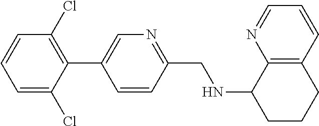 [SMILES: Clc1cccc(Cl)c1-c1ccc(CNC2CCCc3cccnc32)nc1]

The title compound (90 mg, 53%) was prepared in a manner similar to that in Example 6 step 3 from tert-butyl ((5-(2-chloro-6-cyanophenyl)pyridin-2-yl)methyl)carbamate and 6,7-dihydroquinolin-8(5H)-one. LC-MS (M+H)⁺=375.1.

### Step 3: tert-butyl ((7-(1-(((5-(2-chloro-6-cyanophenyl)pyridin-2-yl)methyl)(5,6,7,8-tetrahydroquinolin-8-yl)carbamoyl)cyclopropyl)-4-oxo-3,4-dihydrophthalazin-1-vll)methyl)carbamate

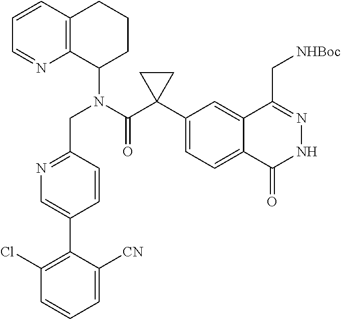 [SMILES: CC(C)(C)OC(=O)NCc1n[nH]c(=O)c2ccc(C3(C(=O)N(Cc4ccc(-c5c(Cl)cccc5C#N)cn4)C4CCCc5cccnc54)CC3)cc12]

The title compound (30 mg, 83%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and 3-chloro-2-(6-(((5,6,7,8-tetrahydroquinolin-8-yl)amino)methyl)pyridin-3-yl)benzonitrile. LC-MS (M+H)⁺=716.2.

### Step 4: 1-(4-(aminomethyl)-1-oxo-1,2-dihydrophthalazin-6-yl)-N-((5-(2-chloro-6-cyanophenyl)pyridin-2-yl)methyl)-N-(5,6,7,8-tetrahydroquinolin-8-yl)cyclopropane-1-carboxamide

Example 13 (8 mg, 32%) was prepared in a manner similar to that in Example 6 step 5 from tert-butyl ((7-(1-(((5-(2-chloro-6-cyanophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.64 (s, 1H), 8.59 (d, J=1.6 Hz, 1H), 8.43 (d, J=4.2 Hz, 1H), 8.28-8.22 (m, 1H), 8.03-7.87 (m, 5H), 7.70-7.61 (m, 1H), 7.50 (t, J=8.4 Hz, 1H), 7.39 (d, J=8.1 Hz, 1H), 7.24-7.19 (m, 1H), 5.56 (dd, J=10.7, 6.0 Hz, 1H), 4.79-4.75 (m, 1H), 4.27-4.21 (m, 2H), 3.66-3.62 (m, 1H), 2.75-2.59 (m, 2H), 1.91 (d, J=4.8 Hz, 1H), 1.78-1.57 (m, 4H), 1.43-1.41 (m, 1H), 1.26 (s, 1H), 0.93 (s, 1H). LC-MS (M+H)⁺=616.2.

### Example 14: 1-(4-(aminomethyl)-1-oxo-1,2-dihydrophthalazin-6-yl)-N-((5-(tert-butyl)pyridin-2-yl)methyl)-N-(5,6,7,8-tetrahydroquinolin-8-yl)cyclopropane-1-carboxamide

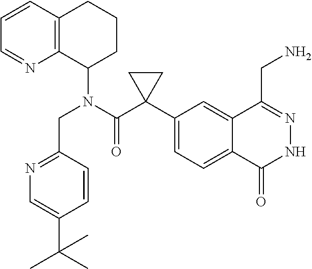 [SMILES: CC(C)(C)c1ccc(CN(C(=O)C2(c3ccc4c(=O)[nH]nc(CN)c4c3)CC2)C2CCCc3cccnc32)nc1]

### Step 1: 5-(tert-butyl)-N-methoxy-N-methylpicolinamide

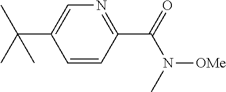 [SMILES: CON(C)C(=O)c1ccc(C(C)(C)C)cn1]

A mixture of 5-tert-butylpyridine-2-carboxylic acid (130 mg, 0.73 mmol), N,O-dimethylhydroxylamine hydrochloride (92 mg, 0.94 mmol), HATU (303 mg, 0.80 mmol) and DIPEA (281 mg, 2.18 mmol) in DMF (3 mL) was stirred at 25° C. for 12 h under nitrogen. The mixture was diluted with water (10 mL) and extracted with EtOAc (5 mL×3). The combined organic layer was washed with brine (10 mL), dried over Na₂SO₄, filtered and concentrated under reduced pressure. The residue was purified by silica gel chromatograph (PE:EtOAc=1:0 to 1:1) to give the title compound (140 mg, 87%). LC-MS (M+H)⁺=223.2.

### Step 2: 5-(tert-butyl)picolinaldehyde

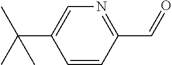

To a solution of 5-(tert-butyl)-N-methoxy-N-methylpicolinamide (100 mg, 0.45 mmol) in anhydrous THF (2 mL) was added DIBAL-H (1.0 M, 0.45 mL, 0.45 mmol) at −78° C. The mixture was stirred at −78° C. for 2 h then quenched with saturated Rochelle salt (5 mL), warmed to 20° C. and stirred for 1 hr. The mixture was extracted with EtOAc (5 mL×3). The combined organic layer was washed with brine (10 mL), dried over Na₂SO₄, filtered and concentrated under reduced pressure. The residue was purified by silica gel chromatograph (PE:EtOAc=1:0 to 5:1) to give the title compound (50 mg, 68%). ¹H NMR (400 MHz, CDCl₃) δ 10.08 (s, 1H), 8.84 (d, J=2.0 Hz, 1H), 7.92 (d, J=8.0 Hz, 1H), 7.87 (dd, J=8.0, 2.0 Hz, 1H), 1.41 (s, 9H).

### Step 3: N-((5-(tert-butyl)pyridin-2-yl)methyl)-5,6,7,8-tetrahydroquinolin-8-amine

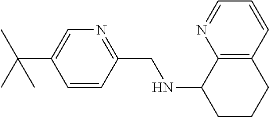 [SMILES: CC(C)(C)c1ccc(CNC2CCCc3cccnc32)nc1]

The title compound (84 mg, 71%) was prepared in a manner similar to that in Example 12 step 4 from 5,6,7,8-tetrahydroquinolin-8-amine and 5-(tert-butyl)picolinaldehyde. LC-MS (M+H)⁺=296.3.

### Step 4: tert-butyl ((7-(1-(((5-(tert-butyl)pyridin-2-yl)methyl)(5,6,7,8-tetrahydroquinolin-8-yl)carbamoyl)cyclopropyl)-4-oxo-3,4-dihydrophthalazin-1-vll)methyl)carbamate

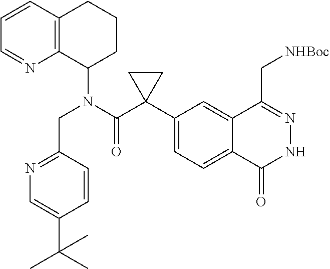 [SMILES: CC(C)(C)OC(=O)NCc1n[nH]c(=O)c2ccc(C3(C(=O)N(Cc4ccc(C(C)(C)C)cn4)C4CCCc5cccnc54)CC3)cc12]

The title compound (50 mg, 47%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(tert-butyl)pyridin-2-yl)methyl)-5,6,7,8-tetrahydroquinolin-8-amine. LC-MS (M+H)⁺=637.2.

### Step 5: 1-(4-(aminomethyl)-1-oxo-1,2-dihydrophthalazin-6-yl)-N-((5-(tert-butyl)pyridin-2-yl)methyl)-N-(5,6,7,8-tetrahydroquinolin-8-yl)cyclopropane-1-carboxamide

Example 14 (36 mg, 85%) was prepared in a manner similar to that in Example 6 step 5 from tert-butyl ((7-(1-(((5-(tert-butyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.95-12.84 (m, 1H), 8.58-8.32 (m, 5H), 8.09-8.04 (m, 1H), 7.87-7.76 (m, 1H), 7.51 (d, J=8.9 Hz, 1H), 7.27-7.13 (m, 1H), 5.56-4.40 (m, 4H), 3.55-3.52 (m, 2H), 2.85-2.57 (m, 2H), 2.20 (s, 1H), 2.04-1.42 (m, 5H), 1.32 (s, 4H), 1.18 (s, 4H), 0.99-0.95 (m, 1H). LC-MS (M+H)⁺=537.2.

### Example 15: 1-(4-(aminomethyl)-1-oxo-1,2-dihydrophthalazin-6-yl)-N-((4-bromoisoquinolin-1-yl)methyl)-N-(5,6,7,8-tetrahydroquinolin-8-yl)cyclopropane-1-carboxamide

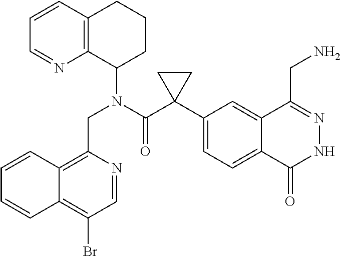 [SMILES: NCc1n[nH]c(=O)c2ccc(C3(C(=O)N(Cc4ncc(Br)c5ccccc45)C4CCCc5cccnc54)CC3)cc12]

### Step 1: 4-bromoisoquinoline 2-oxide

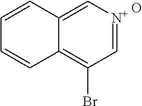 [SMILES: Brc1c[nH+]cc2ccccc12 ; O]

To a solution of 4-bromoisoquinoline (1.0 g, 4.81 mmol) in DCM (10 mL) was added mCPBA (80%, 1.35 g, 6.25 mmol) at 0° C. The mixture was warmed to 25° C. and stirred for 1 h. The mixture was quenched by slow addition of 10% aqueous Na₂S₂O₃ solution (10 mL). The mixture was extracted with DCM (10 mL×3). The combined organic layer was washed with brine (20 mL), dried over Na₂SO₄, filtered and concentrated in vacuo. The residue was purified by silica gel chromatograph (EtOAc:MeOH=1:0 to 10:1) to give the title compound (900 mg, 84%). LC-MS (M+H)⁺=224.0.

### Step 2: 4-bromoisoquinoline-1-carbonitrile

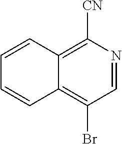 [SMILES: N#Cc1ncc(Br)c2ccccc12]

To a solution of 4-bromoisoquinoline 2-oxide (900 mg, 4.02 mmol) and TMSCN (438 mg, 4.42 mmol) in THF (27 mL) was added DBU (1.37 g, 9.00 mmol). The mixture was stirred at 70° C. for 0.5 h, cooled to room temperature and concentrated under reduced pressure. The residue was purified by silica gel chromatograph (PE:EtOAc=1:0 to 10:1) to give the title compound (800 mg, 85%) was obtained as a white solid. ¹H NMR (400 MHz, CDCl₃) δ 8.86 (s, 1H), 8.38 (d, J=8.4 Hz, 1H), 8.29 (d, J=8.4 Hz, 1H), 7.97 (t, J=8.4 Hz, 1H), 7.89 (t, J=8.4 Hz, 1H).

### Step 3: (4-bromoisoquinolin-1-yl)methanamine

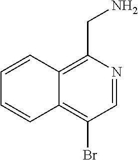 [SMILES: NCc1ncc(Br)c2ccccc12]

Raney-Ni (4 mg, 0.05 mmol) was added to a reaction flask under Ar and AcOH (3 mL) was added. A solution of 4-bromoisoquinoline-1-carbonitrile (100 mg, 0.43 mmol) in AcOH (2 mL) was added. The suspension was degassed under vacuum and purged with hydrogen for three times. The mixture was stirred under hydrogen (50 psi) at 25° C. for 4 h. Solid was filtered off and the filtrate was concentrated under reduced pressure. The residue was purified by prep-HPLC to give the title compound (30 mg, 29%). LC-MS (M+H)⁺=237.1.

### Step 4: N-((4-bromoisoquinolin-1-yl)methyl)-5,6,7,8-tetrahydroquinolin-8-amine

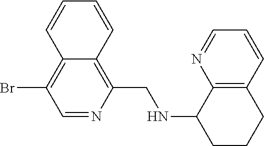 [SMILES: Brc1cnc(CNC2CCCc3cccnc32)c2ccccc12]

The title compound (107 mg, 41%) was prepared in a manner similar to that in Example 3 step 3 from (4-bromoisoquinolin-1-yl)methanamine and 6,7-dihydroquinolin-8(5H)-one. LC-MS (M+H)⁺=367.9.

### Step 5: tert-butyl ((7-(1-(((4-bromoisoquinolin-1-yl)methyl)(5,6,7,8-tetrahydroquinolin-8-yl)carbamoyl)cyclopropyl)-4-oxo-3,4-dihydrophthalazin-1-yl)methyl)carbamate

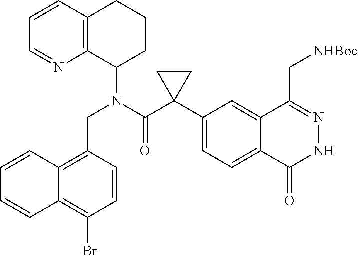 [SMILES: CC(C)(C)OC(=O)NCc1n[nH]c(=O)c2ccc(C3(C(=O)N(Cc4ccc(Br)c5ccccc45)C4CCCc5cccnc54)CC3)cc12]

The title compound (20 mg, 33%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((4-bromoisoquinolin-1-yl)methyl)-5,6,7,8-tetrahydroquinolin-8-amine. LC-MS (M+H)⁺=709.4.

### Step 6: 1-(4-(aminomethyl)-1-oxo-1,2-dihydrophthalazin-6-yl)-N-((4-bromoisoquinolin-1-yl)methyl)-N-(5,6,7,8-tetrahydroquinolin-8-yl)cyclopropane-1-carboxamide

Example 15 (5 mg, 29%) was prepared in a manner similar to that in Example 6 step 5 from tert-butyl ((7-(1-(((4-bromoisoquinolin-1-yl)methyl)(5,6,7,8-tetrahydroquinolin-8-yl)carbamoyl)cyclopropyl)-4-oxo-3,4-dihydrophthalazin-1-yl)methyl). ¹H NMR (400 MHz, DMSO-d6) δ 12.5 (s, 1H), 8.83 (s, 1H), 8.44-8.35 (m, 2H), 8.19-8.13 (m, 2H), 7.97-7.56 (m, 4H), 7.49-7.12 (m, 2H), 5.49-5.26 (m, 2H), 4.11-4.07 (m, 3H), 2.68-2.53 (m, 3H), 2.04-1.24 (m, 8H), 0.94-0.85 (m, 1H). LC-MS (M+H)⁺=609.3.

### Example 16: 1-(4-(aminomethyl)-1-oxo-1,2-dihydrophthalazin-6-yl)-N-((5-(1-methyl-1H-pyrazol-5-yl)pyridin-2-yl)methyl)-N-(5,6,7,8-tetrahydroquinolin-8-yl)cyclopropane-1-carboxamide

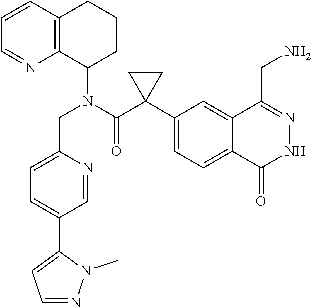 [SMILES: Cn1nccc1-c1ccc(CN(C(=O)C2(c3ccc4c(=O)[nH]nc(CN)c4c3)CC2)C2CCCc3cccnc32)nc1]

### Step 1: tert-butyl ((5-(1-methyl-1H-pyrazol-5-yl)pyridin-2-yl)methyl)carbamate

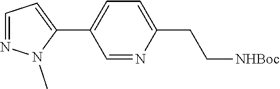 [SMILES: Cn1nccc1-c1ccc(CCNC(=O)OC(C)(C)C)nc1]

The title compound (150 mg, 36%) was prepared in a manner similar to that in Example 3 step 1 from tert-butyl ((5-bromopyridin-2-yl)methyl)carbamate and 1-methyl-5-(4,4,5,5-tetramethyl-1,3,2-dioxaborolan-2-yl)-1H-pyrazole. LC-MS (M+H)⁺=289.3.

### Step 2: N-((5-(1-methyl-1H-pyrazol-5-yl)pyridin-2-yl)methyl)-5,6,7,8-tetrahydroquinolin-8-amine

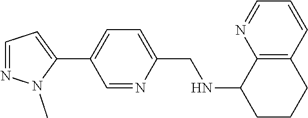 [SMILES: Cn1nccc1-c1ccc(CNC2CCCc3cccnc32)nc1]

The title compound (104 mg, 44%) was prepared in a manner similar to that in Example 6 step 3 from tert-butyl ((5-(1-methyl-1H-pyrazol-5-yl)pyridin-2-yl)methyl)carbamate and 6,7-dihydroquinolin-8(5H)-one. LC-MS (M+H)⁺=320.2.

### Step 3: tert-butyl ((7-(1-(((5-(1-methyl-1H-pyrazol-5-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

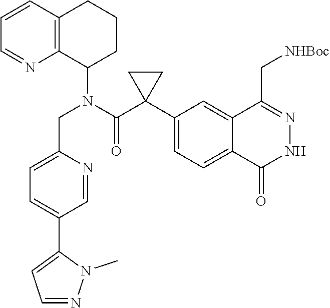 [SMILES: Cn1nccc1-c1ccc(CN(C(=O)C2(c3ccc4c(=O)[nH]nc(CNC(=O)OC(C)(C)C)c4c3)CC2)C2CCCc3cccnc32)nc1]

The title compound (30 mg, 65%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(1-methyl-1H-pyrazol-5-yl)pyridin-2-yl)methyl)-5,6,7,8-tetrahydroquinolin-8-amine. LC-MS (M+H)⁺=661.3.

### Step 4: 1-(4-(aminomethyl)-1-oxo-1,2-dihydrophthalazin-6-yl)-N-((5-(1-methyl-1H-pyrazol-5-yl)pyridin-2-yl)methyl)-N-(5,6,7,8-tetrahydroquinolin-8-yl)cyclopropane-1-carboxamide

Example 16 (8 mg, 17%) was prepared in a manner similar to that in Example 6 step 5 from tert-butyl ((7-(1-(((5-(1-methyl-1H-pyrazol-5-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.72 (s, 1H), 8.68 (d, J=1.9 Hz, 1H), 8.43 (d, J=3.0 Hz, 1H), 8.33-8.25 (m, 2H), 8.00-7.89 (m, 3H), 7.58-7.41 (m, 3H), 7.38-7.16 (m, 2H), 6.53 (d, J=1.9 Hz, 1H), 5.56-5.53 (m, 1H), 4.75-4.72 (m, 1H), 4.32-4.24 (m, 2H), 3.89 (s, 3H), 3.72 (s, 1H), 3.60-3.58 (m, 1H), 2.72-2.66 (m, 2H), 2.54 (s, 2H), 1.93-1.89 (m, 1H), 1.78-1.66 (m, 2H), 1.59-1.55 (m, 2H), 1.44-1.41 (m, 1H), 1.26 (s, 1H), 0.97-0.89 (m, 1H). LC-MS (M+H)⁺=561.3.

### Example 17: 1-(4-(aminomethyl)-1-oxo-1,2-dihydrophthalazin-6-yl)-N-(4-methoxy-5,6,7,8-tetrahydroquinolin-8-yl)-N-((5-(trifluoromethyl)pyridin-2-yl)methyl)cyclopropane-1-carboxamide

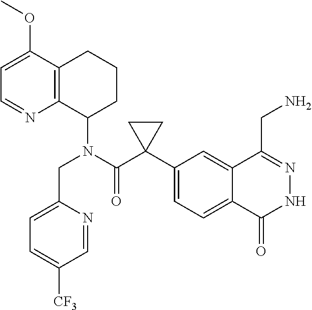 [SMILES: COc1ccnc2c1CCCC2N(Cc1ccc(C(F)(F)F)cn1)C(=O)C1(c2ccc3c(=O)[nH]nc(CN)c3c2)CC1]

### Step 1: 4-methoxy-N-((5-(trifluoromethyl)pyridin-2-yl)methyl)-5,6,7,8-tetrahydroquinolin-8-amine

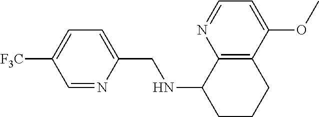 [SMILES: COc1ccnc2c1CCCC2NCc1ccc(C(F)(F)F)cn1]

The title compound (60 mg, 29%) was prepared in a manner similar to that in Example 3 step 3 from (5-(trifluoromethyl)pyridin-2-yl)methanamine and 4-methoxy-6,7-dihydroquinolin-8(5H)-one. LC-MS (M+H)⁺=338.1.

### Step 2: tert-butyl ((7-(1-((4-methoxy-5,6,7,8-tetrahydroquinolin-8-yl)((5-(trifluoromethyl)pyridin-2-yl)methyl)carbamoyl)cyclopropyl)-4-oxo-3A-dihydrophthalazin-1-yl)methyl)carbamate

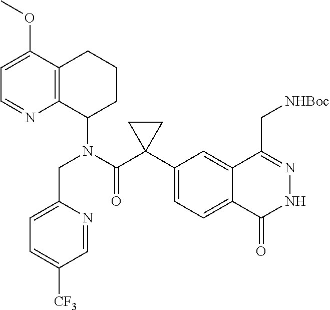 [SMILES: COc1ccnc2c1CCCC2N(Cc1ccc(C(F)(F)F)cn1)C(=O)C1(c2ccc3c(=O)[nH]nc(CNC(=O)OC(C)(C)C)c3c2)CC1]

The title compound (30 mg, 71%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and 4-methoxy-N-((5-(trifluoromethyl)pyridin-2-yl)methyl)-5,6,7,8-tetrahydroquinolin-8-amine. LC-MS (M+H)⁺=679.3.

### Step 3: 1-(4-(aminomethyl)-1-oxo-1,2-dihydrophthalazin-6-yl)-N-(4-methoxy-5,6,7,8-tetrahydroquinolin-8-yl)-N-((5-(trifluoromethyl)pyridin-2-yl)methyl)cyclopropane-1-carboxamide

Example 17 (13 mg, 51%) was prepared in a manner similar to that in Example 6 step 5 from tert-butyl ((7-(1-((4-methoxy-5,6,7,8-tetrahydroquinolin-8-yl)((5-(trifluoromethyl)pyridin-2-yl)methyl)carbamoyl)cyclopropyl)-4-oxo-3,4-dihydrophthalazin-1-yl)methyl)carbamate. ¹H NMR (500 MHz, DMSO-d6) δ 12.98-12.58 (m, 1H), 8.42-8.29 (m, 2H), 8.26-7.94 (m, 3H), 7.84-7.12 (m, 3H), 6.95-6.85 (m, 1H), 5.55-5.28 (m, 1H), 4.93-4.61 (m, 1H), 4.47-4.20 (m, 2H), 3.90-3.78 (m, 3H), 3.62-3.49 (m, 2H), 2.68-2.54 (m, 3H), 2.43-1.87 (m, 3H), 1.75-0.80 (m, 9H).

### Example 18: 1-(4-(aminomethyl)-1-oxo-1,2-dihydrophthalazin-6-yl)-N-((5-(2,6-difluorophenyl)pyridin-2-yl)methyl)-N-(5,6,7,8-tetrahydroquinolin-8-yl)cyclopropane-1-carboxamide

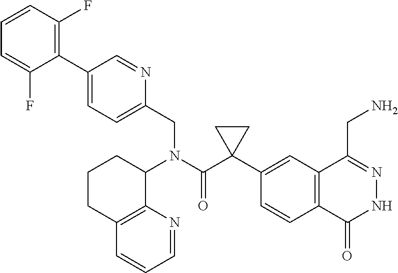 [SMILES: NCc1n[nH]c(=O)c2ccc(C3(C(=O)N(Cc4ccc(-c5c(F)cccc5F)cn4)C4CCCc5cccnc54)CC3)cc12]

### Step 1: tert-butyl ((5-(2,6-difluorophenyl)pyridin-2-yl)methyl)carbamate

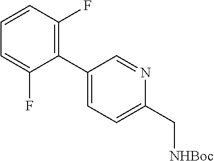 [SMILES: CC(C)(C)OC(=O)NCc1ccc(-c2c(F)cccc2F)cn1]

To a solution of tert-butyl ((5-bromopyridin-2-yl)methyl)carbamate (5.0 g, 17.4 mmol) in dioxane (50 mL) was added BPD (5.31 g, 20.9 mmol), Pd(dppf)Cl₂ (1.27 g, 1.74 mmol) and KOAc (4.27 g, 43.5 mmol). The mixture was stirred at 85° C. for 12 h. The mixture was cooled to room temperature, filtered and concentrated under reduced pressure. The crude was re-dissolved in dioxane (25 mL), toluene (12 mL) and water (12 mL) followed by addition of 1,3-difluoro-2-iodobenzene (4.28 g, 17.8 mmol), K₃PO₄ (6.32 g, 29.8 mmol) and Pd(dppf)Cl₂ (871 mg, 1.19 mmol). The mixture was stirred at 85° C. for 3 h. The mixture was cooled to room temperature, diluted with water (30 mL) and extracted with EtOAc (20 mL×3). The combined organic layer was washed with brine (10 mL×2), dried over Na₂SO₄, filtered and concentrated under reduced pressure. The residue was purified by silica gel chromatograph (PE:EtOAc=1:0 to 5:1) to give the title compound (3.0 g, 54%). ¹H NMR (400 MHz, CDCl₃) δ 8.64 (s, 1H), 7.78 (d, J=8.4 Hz, 1H), 7.39 (d, J=8.4 Hz, 1H), 7.36-7.28 (m, 1H), 7.06-6.96 (m, 1H), 5.62 (br s, 1H), 4.50 (d, J=5.6 Hz, 2H), 7.47 (s, 9H). LC-MS (M+H)⁺=321.1.

### Step 2: (5-(2,6-difluorophenyl)pyridin-2-yl)methanamine

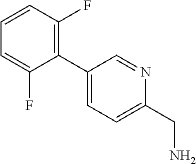 [SMILES: NCc1ccc(-c2c(F)cccc2F)cn1]

A mixture of tert-butyl ((5-(2,6-difluorophenyl)pyridin-2-yl)methyl)carbamate (3.0 g, 9.37 mmol) in methanolic HCl (4 M, 50 mL) was stirred at 25° C. for 2 h, then the mixture was neutralized with methanolic NaOH (1 M) to pH 7. The mixture was concentrated to dryness under reduced pressure, and the residue was triturated with DCM/MeOH (10/1, 10 mL). The filtrate was collected by filtration and concentrated under vacuum to give the title compound (1.5 g, 73%). ¹H NMR (400 MHz, CD₃OD) δ 8.72 (s, 1H), 7.97 (d, J=6.0 Hz, 1H), 7.60 (d, J=6.0 Hz, 1H), 7.54-7.45 (m, 1H), 7.20-7.10 (m, 1H), 4.37 (s, 2H). LC-MS (M+H)⁺=221.1.

### Step 3: N-((5-(2,6-difluorophenyl)pyridin-2-yl)methyl)-5,6,7,8-tetrahydroquinolin-8-amine

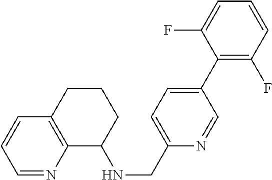 [SMILES: Fc1cccc(F)c1-c1ccc(CNC2CCCc3cccnc32)nc1]

To a solution of 6,7-dihydroquinolin-8(5H)-one (0.25 g, 1.70 mmol) and (5-(2,6-difluorophenyl)pyridin-2-yl)methanamine (449 mg, 2.04 mmol) in DCM (4 mL) and MeOH (0.4 mL) was added NaBH(OAc)₃ (720 mg, 3.40 mmol). The mixture was stirred at 25° C. for 2 h then slowly poured into saturated NaHCO₃ (4.0 mL). The mixture was extracted with DCM (3.0 mL×2). The combined organic layer was washed with brine (3.0 mL), dried with Na₂SO₄, filtered and concentrated under vacuum. The residue was purified by prep-HPLC to give the title compound (176 mg, 30%). LC-MS (M+H)⁺=352.1.

### Step 4: tert-butyl ((7-(1-(((5-(2,6-difluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

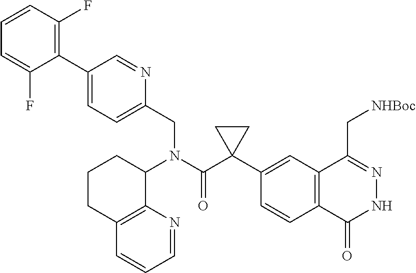 [SMILES: CC(C)(C)OC(=O)NCc1n[nH]c(=O)c2ccc(C3(C(=O)N(Cc4ccc(-c5c(F)cccc5F)cn4)C4CCCc5cccnc54)CC3)cc12]

To a solution of 1-(4-(((tert-butoxycarbonyl)amino)methyl)-1-oxo-1,2-dihydrophthalazin-6-yl)cyclopropane-1-carboxylic acid (155 mg, 0.43 mmol) in DMF (2.5 mL) was added HATU (180 mg, 0.47 mmol), N-((5-(2,6-difluorophenyl)pyridin-2-yl)methyl)-5,6,7,8-tetrahydroquinolin-8-amine (166 mg, 0.47 mmol) and DIPEA (111 mg, 0.86 mmol). The solution was stirred for 6 h at 50° C. The mixture was cooled to room temperature, poured into water (100 mL) and then extracted with EtOAc (100 mL). The combined organic layer was washed with brine (100 mL×2), dried over Na₂SO₄, filtered and concentrated under vacuum. The residue was purified by silica gel chromatograph (DCM:MeOH=20:1) to give the title compound (200 mg, 67%). LC-MS (M+H)⁺=693.4.

### Step 5: 1-(4-(aminomethyl)-1-oxo-1,2-dihydrophthalazin-6-yl)-N-((5-(2,6-difluorophenyl)pyridin-2-yl)methyl)-N-(5,6,7,8-tetrahydroquinolin-8-yl)cyclopropane-1-carboxamide

To a solution of tert-butyl ((7-(1-(((5-(2,6-difluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate (200 mg, 0.29 mmol) in DCM (4 mL), was added TFA (1 mL). The mixture was stirred for 1 h at room temperature then concentrated under vacuum. The residue was purified by prep-HPLC to give Example 18 (149 mg, 87%). ¹H NMR (500 MHz, DMSO-d6) δ 13.05-12.73 (m, 1H), 8.64-8.31 (m, 5H), 8.20-8.04 (m, 1H), 7.93-7.47 (m, 4H), 7.35-7.18 (m, 3H), 5.73-5.35 (m, 1H), 5.02-4.29 (m, 3H), 3.71-3.59 (m, 1H), 2.88-2.56 (m, 3H), 2.24-0.94 (m, 8H).

### Example 19A&19B: (R)-1-(4-(aminomethyl)-1-oxo-1,2-dihydrophthalazin-6-yl)-N-((5-(2,6-difluorophenyl)pyridin-2-yl)methyl)-N-(5,6,7,8-tetrahydroquinolin-8-yl)cyclopropane-1-carboxamide & (S)-1-(4-(aminomethyl)-1-oxo-1,2-dihydrophthalazin-6-yl)-N-((5-(2,6-difluorophenyl)pyridin-2-yl)methyl)-N-(5,6,7,8-tetrahydroquinolin-8-yl)cyclopropane-1-carboxamide

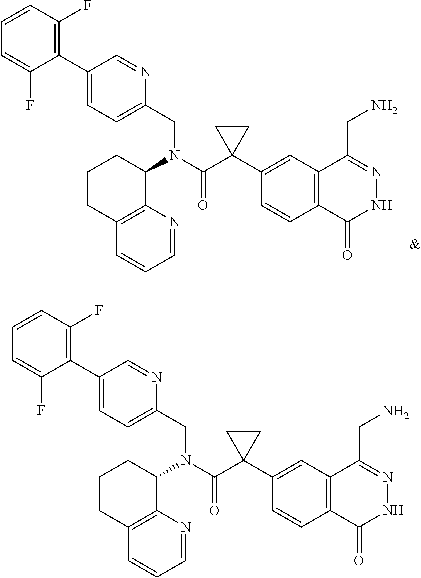 [SMILES: NCc1n[nH]c(=O)c2ccc(C3(C(=O)N(Cc4ccc(-c5c(F)cccc5F)cn4)[C@@H]4CCCc5cccnc54)CC3)cc12 ; NCc1n[nH]c(=O)c2ccc(C3(C(=O)N(Cc4ccc(-c5c(F)cccc5F)cn4)[C@H]4CCCc5cccnc54)CC3)cc12]

Example 18 (146 mg) was separated by chiral prep-HPLC to give Example 19A (53 mg, 36%) and Example 19B (48 mg, 33%). Analytical chiral-HPLC condition as below. Column: CHIRALPAK IC-3; Column size: 4.6×50 mm, 3 μm; Mobile phase: (Hexane:DCM=1:1, with 0.1% diethylamine):EtOH=75:25; Flow: 1 mL/min; Temperature: 25° C.

Example 19A: Chiral HPLC t_{R}=2.97 min. ¹H NMR (500 MHz, DMSO-d6) δ 12.83-12.42 (m, 1H), 8.71-8.40 (m, 2H), 8.34-7.69 (m, 4H), 7.62-7.44 (m, 2H), 7.39-7.13 (m, 4H), 5.72-5.26 (m, 1H), 5.17-4.39 (m, 2H), 4.30-4.13 (m, 2H), 3.73-3.54 (m, 1H), 2.87-2.59 (m, 2H), 2.21-0.88 (m, 8H). LC-MS (M+H)⁺=593.4;

Example 19B: Chiral HPLC t_{R}=4.13 min. ¹H NMR (500 MHz, DMSO-d6) δ 12.83-12.42 (m, 1H), 8.71-8.40 (m, 2H), 8.34-7.69 (m, 4H), 7.62-7.44 (m, 2H), 7.39-7.13 (m, 4H), 5.72-5.26 (m, 1H), 5.17-4.39 (m, 2H), 4.30-4.13 (m, 2H), 3.73-3.54 (m, 1H), 2.87-2.59 (m, 2H), 2.21-0.88 (m, 8H). LC-MS (M+H)⁺=593.4.

### Example 20: 1-(4-(aminomethyl)-1-oxo-1,2-dihydrophthalazin-6-yl)-N-((5-phenoxypyridin-2-yl)methyl)-N-(5,6,7,8-tetrahydroquinolin-8-yl)cyclopropane-1-carboxamide

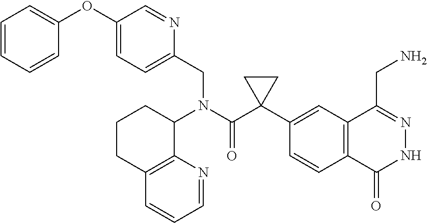 [SMILES: NCc1n[nH]c(=O)c2ccc(C3(C(=O)N(Cc4ccc(Oc5ccccc5)cn4)C4CCCc5cccnc54)CC3)cc12]

### Step 1: 5-phenoxypicolinaldehyde

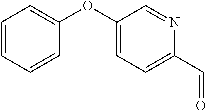 [SMILES: O=Cc1ccc(Oc2ccccc2)cn1]

To a solution of 5-fluoropicolinaldehyde (0.20 g, 1.60 mmol) in DMF (2 mL) was added phenol (181 mg, 1.92 mmol) and K₂CO₃ (663 mg, 4.80 mmol) at room temperature. The mixture was heated to 110° C. and stirred for 1 h. The mixture was cooled to room temperature partitioned between EtOAc (80 mL) and water (40 mL). The organic phase was separated, dried over Na₂SO₄, filtered and concentrated under vacuum. The residue was purified by silica gel chromatograph (PE:EtOAc=1:0 to 1:1) to give the title compound (0.27 g, 85%). LC-MS (M+H)⁺=200.2.

### Step 2: N-((5-phenoxypyridin-2-yl)methyl)-5,6,7,8-tetrahydroquinolin-8-amine

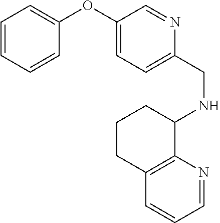 [SMILES: c1ccc(Oc2ccc(CNC3CCCc4cccnc43)nc2)cc1]

The title compound (316 mg, 70%) was prepared in a manner similar to that in Example 12 step 4 from 5,6,7,8-tetrahydroquinolin-8-amine and 5-phenoxypicolinaldehyde. LC-MS (M+H)⁺=332.2.

### Step 3: tert-butyl ((4-oxo-7-(1-(((5-phenoxypyridin-2-yl)methyl)(5,6,7,8-tetrahydroquinolin-8-yl)carbamoyl)cyclopropyl)-3,4-dihydrophthalazin-1-yl)methyl)carbamate

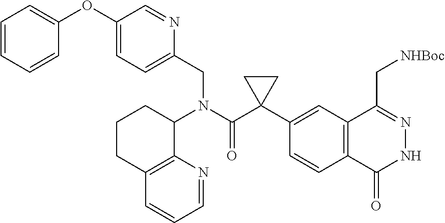 [SMILES: CC(C)(C)OC(=O)NCc1n[nH]c(=O)c2ccc(C3(C(=O)N(Cc4ccc(Oc5ccccc5)cn4)C4CCCc5cccnc54)CC3)cc12]

The title compound (10 mg, 24%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-phenoxypyridin-2-yl)methyl)-5,6,7,8-tetrahydroquinolin-8-amine. LC-MS (M+H)⁺=673.4.

### Step 4: 1-(4-(aminomethyl)-1-oxo-1,2-dihydrophthalazin-6-yl)-N-((5-phenoxypyridin-2-yl)methyl)-N-(5,6,7,8-tetrahydroquinolin-8-yl)cyclopropane-1-carboxamide

Example 20 (2 mg, 23%) was prepared in a manner similar to that in Example 6 step 4 from tert-butyl ((4-oxo-7-(1-(((5-phenoxypyridin-2-yl)methyl)(5,6,7,8-tetrahydroquinolin-8-yl)carbamoyl)cyclopropyl)-3,4-dihydrophthalazin-1-yl)methyl)carbamate. ¹H NMR (500 MHz, DMSO-d6) δ 12.60-12.37 (m, 1H), 8.49-8.36 (m, 1H), 8.31-8.05 (m, 2H), 8.00-7.68 (m, 2H), 7.54-7.30 (m, 4H), 7.29-7.13 (m, 3H), 7.09-6.82 (m, 2H), 5.60-5.26 (m, 1H), 4.89-4.66 (m, 1H), 4.10-3.92 (m, 2H), 3.55-3.37 (m, 1H), 2.81-2.57 (m, 2H), 2.03-0.81 (m, 10H). LC-MS (M+H)⁺=573.4.

### Example 21: 1-(4-(aminomethyl)-1-oxo-1,2-dihydrophthalazin-6-yl)-N-((5-(2,6-difluorophenyl)pyridin-2-yl)methyl)-N-(8-fluoro-1,2,3,4-tetrahydronaphthalen-1-yl)cyclopropane-1-carboxamide

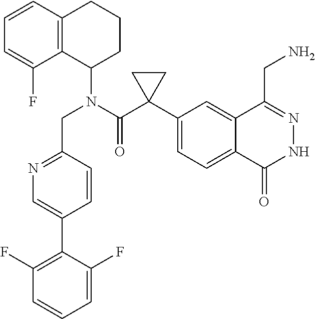 [SMILES: NCc1n[nH]c(=O)c2ccc(C3(C(=O)N(Cc4ccc(-c5c(F)cccc5F)cn4)C4CCCc5cccc(F)c54)CC3)cc12]

### Step 1: N-((5-bromopyridin-2-yl)methyl)-8-fluoro-1,2,3,4-tetrahydronaphthalen-1-amine

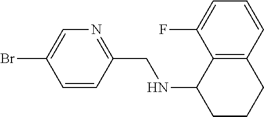 [SMILES: Fc1cccc2c1C(NCc1ccc(Br)cn1)CCC2]

To a solution of 8-fluoro-3,4-dihydronaphthalen-1(2H)-one (100 mg, 0.61 mmol) and (5-bromopyridin-2-yl)methanamine (125 mg, 0.67 mmol) in dioxane (3 mL) was added Ti(OEt)₄ (695 mg, 3.05 mmol). The mixture was warmed to 60° C. and stirred for 2 h. The mixture was cooled to room temperature and NaBH(OAc)₃ (258 mg, 1.22 mmol) was added. After 2 h, the mixture was diluted with water (3 mL) and extracted with EtOAc (2 mL×3). The combined organic layer was washed with brine (2 mL×2), dried over Na₂SO₄, filtered and concentrated under reduced pressure. The residue was purified by silica gel chromatograph (PE:EtOAc=1:0 to 5:1) to give the title compound (50 mg, 24%). LC-MS (M+H)⁺=335.1.

### Step 2: N-((5-(2,6-difluorophenyl)pyridin-2-yl)methyl)-8-fluoro-1,2,3,4-tetrahydronaphthalen-1-amine

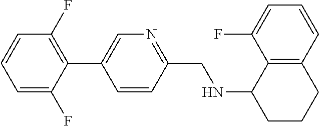 [SMILES: Fc1cccc(F)c1-c1ccc(CNC2CCCc3cccc(F)c32)nc1]

The title compound (26 mg, 47%) was prepared in a manner similar to that in Example 3 step 1 from N-((5-bromopyridin-2-yl)methyl)-8-fluoro-1,2,3,4-tetrahydronaphthalen-1-amine and (2,6-difluorophenyl)boronic acid. LC-MS (M+H)⁺=369.1.

### Step 3: tert-butyl ((7-(1-(((5-(2,6-difluorophenyl)pyridin-2-yl)methyl)(8-fluoro-1,2,3,4-tetrahydronaphthalen-1-yl)carbamoyl)cyclopropyl)-4-oxo-3,4-dihydrophthalazin-1-vll)methyl)carbamate

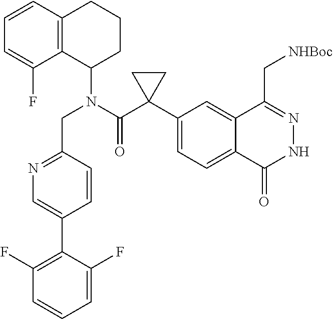 [SMILES: CC(C)(C)OC(=O)NCc1n[nH]c(=O)c2ccc(C3(C(=O)N(Cc4ccc(-c5c(F)cccc5F)cn4)C4CCCc5cccc(F)c54)CC3)cc12]

The title compound (7 mg, 18%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(2,6-difluorophenyl)pyridin-2-yl)methyl)-8-fluoro-1,2,3,4-tetrahydronaphthalen-1-amine. LC-MS (M+H)⁺=710.4.

### Step 4: 1-(4-(aminomethyl)-1-oxo-1,2-dihydrophthalazin-6-yl)-N-((5-(2,6-difluorophenyl)pyridin-2-yl)methyl)-N-(8-fluoro-1,2,3,4-tetrahydronaphthalen-1-yl)cyclopropane-1-carboxamide

Example 21 (1 mg, 17%) was prepared in a manner similar to that in Example 6 step 4 from tert-butyl ((7-(1-(((5-(2,6-difluorophenyl)pyridin-2-yl)methyl)(8-fluoro-1,2,3,4-tetrahydronaphthalen-1-yl)carbamoyl)cyclopropyl)-4-oxo-3,4-dihydrophthalazin-1-yl)methyl)carbamate. ¹H NMR (500 MHz, DMSO-d6) δ 12.77-12.30 (m, 1H), 8.59-8.41 (m, 1H), 8.36-8.11 (m, 1H), 8.10-7.47 (m, 4H), 7.34-7.10 (m, 4H), 7.04-6.81 (m, 2H), 5.79-5.37 (m, 1H), 4.66-4.49 (m, 1H), 4.15-3.98 (m, 2H), 3.89-3.74 (m, 1H), 2.67-2.54 (m, 2H), 2.11-0.72 (m, 10H). LC-MS (M+H)⁺=610.4.

### Example 22: 1-(4-(aminomethyl)-1-oxo-1,2-dihydrophthalazin-6-yl)-N-(4-bromo-2-fluorobenzyl)-N-(5,6,7,8-tetrahydroquinolin-8-yl)cyclopropane-1-carboxamide

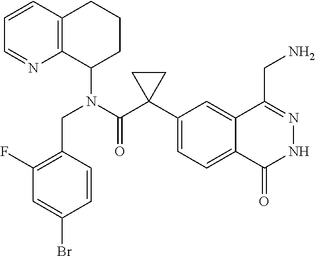 [SMILES: NCc1n[nH]c(=O)c2ccc(C3(C(=O)N(Cc4ccc(Br)cc4F)C4CCCc5cccnc54)CC3)cc12]

### Step 1: N-(4-bromo-2-fluorobenzyl)-5,6,7,8-tetrahydroquinolin-8-amine

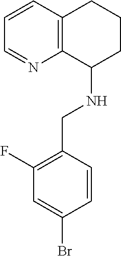 [SMILES: Fc1cc(Br)ccc1CNC1CCCc2cccnc21]

The title compound (74 mg, 33%) was prepared in a manner similar to that in Example 12 step 4 from 5,6,7,8-tetrahydroquinolin-8-amine and 4-bromo-2-fluorobenzaldehyde. LC-MS (M+H)⁺=335.0.

### Step 2: tert-butyl ((7-(1-((4-bromo-2-fluorobenzyl)(5,6,7,8-tetrahydroquinolin-8-yl)carbamoyl)cyclopropyl)-4-oxo-3,4-dihydrophthalazin-1-yl)methyl)carbamate

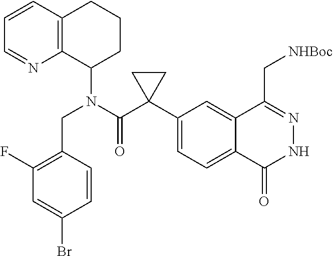 [SMILES: CC(C)(C)OC(=O)NCc1n[nH]c(=O)c2ccc(C3(C(=O)N(Cc4ccc(Br)cc4F)C4CCCc5cccnc54)CC3)cc12]

The title compound (30 mg, 44%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-(4-bromo-2-fluorobenzyl)-5,6,7,8-tetrahydroquinolin-8-amine. LC-MS (M+H)⁺=676.4.

### Step 3: 1-(4-(aminomethyl)-1-oxo-1,2-dihydrophthalazin-6-yl)-N-(4-bromo-2-fluorobenzyl)-N-(5,6,7,8-tetrahydroquinolin-8-yl)cyclopropane-1-carboxamide

Example 22 (8 mg, 31%) was prepared in a manner similar to that in Example 6 step 4 from tert-butyl ((7-(1-((4-bromo-2-fluorobenzyl)(5,6,7,8-tetrahydroquinolin-8-yl)carbamoyl)cyclopropyl)-4-oxo-3,4-dihydrophthalazin-1-yl)methyl)carbamate. ¹H NMR (400 MHz, DMSO-d6) δ 12.45 (s, 1H), 8.48-8.04 (m, 2H), 7.97-7.69 (m, 2H), 7.51-6.96 (m, 5H), 5.60-4.53 (m, 2H), 4.08-4.00 (m, 2H), 3.42-3.39 (m, 1H), 2.73-2.57 (m, 2H), 1.92-0.85 (m, 8H). LC-MS (M+H)⁺=576.4.

### Example 23: 1-(4-(aminomethyl)-1-oxo-1,2-dihydrophthalazin-6-yl)-N-((5-(3,5-dimethyl-1H-pyrazol-4-yl)pyridin-2-yl)methyl)-N-(5,6,7,8-tetrahydroquinolin-8-yl)cyclopropane-1-carboxamide

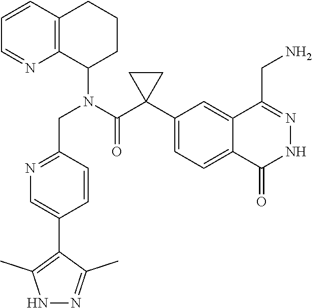 [SMILES: Cc1n[nH]c(C)c1-c1ccc(CN(C(=O)C2(c3ccc4c(=O)[nH]nc(CN)c4c3)CC2)C2CCCc3cccnc32)nc1]

### Step 1: N-((5-bromopyridin-2-yl)methyl)-5,6,7,8-tetrahydroquinolin-8-amine

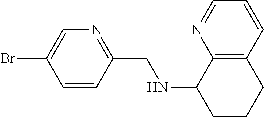 [SMILES: Brc1ccc(CNC2CCCc3cccnc32)nc1]

The title compound (3.0 g, 50%) was prepared in a manner similar to that in Example 18 step 3 from 6,7-dihydroquinolin-8(5H)-one and (5-bromopyridin-2-yl)methanamine. LC-MS (M+H)⁺=318.1.

### Step 2: N-((5-(3,5-dimethyl-1-(tetrahydro-2H-pyran-2-yl)-1H-pyrazol-4-yl)pyridin-2-yl)methyl)-5,6,7,8-tetrahydroquinolin-8-amine

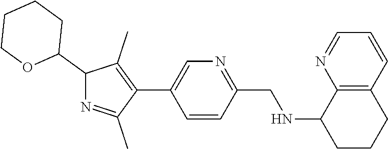 [SMILES: CC1=NC(C2CCCCO2)C(C)=C1c1ccc(CNC2CCCc3cccnc32)nc1]

The title compound (104 mg, 26%) was prepared in a manner similar to that in Example 3 step 1 from N-((5-bromopyridin-2-yl)methyl)-5,6,7,8-tetrahydroquinolin-8-amine and 3,5-dimethyl-1-(tetrahydro-2H-pyran-2-yl)-4-(4,4,5,5-tetramethyl-1,3,2-dioxaborolan-2-yl)-1H-pyrazole. LC-MS (M+H)⁺=418.2.

### Step 3: tert-butyl ((7-(1-(((5-(3,5-dimethyl-1-(tetrahydro-2H-pyran-2-yl)-1H-pyrazol-4-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

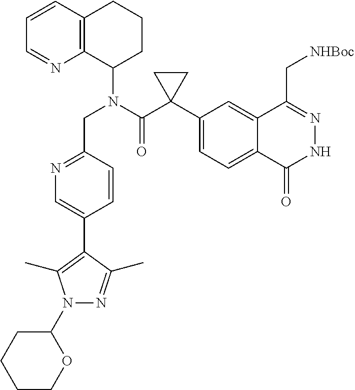 [SMILES: Cc1nn(C2CCCCO2)c(C)c1-c1ccc(CN(C(=O)C2(c3ccc4c(=O)[nH]nc(CNC(=O)OC(C)(C)C)c4c3)CC2)C2CCCc3cccnc32)nc1]

The title compound (30 mg, 48%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(3,5-dimethyl-1-(tetrahydro-2H-pyran-2-yl)-1H-pyrazol-4-yl)pyridin-2-yl)methyl)-5,6,7,8-tetrahydroquinolin-8-amine. LC-MS (M+H)⁺=759.1.

### Step 4: 1-(4-(aminomethyl)-1-oxo-1,2-dihydrophthalazin-6-yl)-N-((5-(3,5-dimethyl-1H-pyrazol-4-yl)pyridin-2-yl)methyl)-N-(5,6,7,8-tetrahydroquinolin-8-yl)cyclopropane-1-carboxamide

Example 23 (16 mg, 70%) was prepared in a manner similar to that in Example 6 step 4 from tert-butyl ((7-(1-(((5-(3,5-dimethyl-1-(tetrahydro-2H-pyran-2-yl)-1H-pyrazol-4-yl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.58-12.26 (m, 2H), 8.57-8.37 (m, 2H), 8.34-8.00 (m, 2H), 7.99-7.62 (m, 2H), 7.55-7.46 (m, 1H), 7.34-7.14 (m, 2H), 5.64-4.57 (m, 2H), 4.16-3.93 (m, 2H), 3.64-3.48 (m, 1H), 2.86-2.60 (m, 2H), 2.34-1.97 (m, 8H), 1.99-1.31 (m, 7H), 0.98-0.84 (m, 1H). LC-MS (M+H)⁺=575.4.

### Example 24: (±)-4-(aminomethyl)-6-(1-((2S,6S)-2-methyl-6-(5-(trifluoromethyl)pyridin-2-yl)piperidine-1-carbonyl)cyclopropyl)phthalazin-1(2H)-one

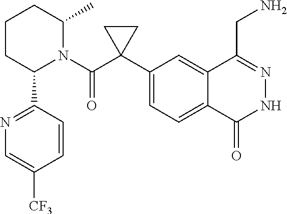 [SMILES: C[C@H]1CCC[C@@H](c2ccc(C(F)(F)F)cn2)N1C(=O)C1(c2ccc3c(=O)[nH]nc(CN)c3c2)CC1]

### Step 1: tert-butyl 2-methyl-6-(5-(trifluoromethyl)pyridin-2-yl)piperidine-1-carboxylate

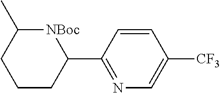 [SMILES: CC1CCCC(c2ccc(C(F)(F)F)cn2)N1C(=O)OC(C)(C)C]

Four identical reactions as described below were set up in parallel. A mixture of 2-chloro-5-(trifluoromethyl)pyridine (75 mg, 0.41 mmol), 1-(tert-butoxycarbonyl)-6-methylpiperidine-2-carboxylic acid (100 mg, 0.41 mmol), nickel(II) chloride ethylene glycol dimethyl ether complex (9 mg, 0.041 mmol), Cs₂CO₃ (335 mg, 1.0 mmol), dtbbpy (17 mg, 0.062 mmol), iridium(III) bis[2-(2,4-difluorophenyl)-5-methylpyridine-N,C20]-4,40-di-tert-butyl-2,20-bipyridine hexafluorophosphate (4.2 mg, 0.004 mmol) in DMF (2 mL) was degassed and purged with nitrogen for 3 times, and then the mixture was stirred for 12 h under irradiation of blue LED (34 W, 455 nm).

The mixture from all four setups were combined then diluted with water (15 mL) and extracted with EtOAc (10 mL×3). The combined organic layer was washed with brine (20 mL), dried over Na₂SO₄, filtered and concentrated under reduced pressure. The residue was purified by silica gel chromatograph (PE:EtOAc=1:0 to 10:1) to give the title compound (75 mg, 13%). LC-MS (M+H)⁺=345.3.

### Step 2: (f)-2-((2S,6S)-6-methylpiperidin-2-yl)-5-(trifluoromethyl)pyridine

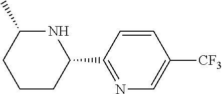 [SMILES: C[C@H]1CCC[C@@H](c2ccc(C(F)(F)F)cn2)N1]

A solution of tert-butyl 2-methyl-6-(5-(trifluoromethyl)pyridin-2-yl)piperidine-1-carboxylate (100 mg, 0.29 mmol) in methanolic HCl (4 M, 1 mL) was stirred at 25° C. for 0.5 h. The mixture was concentrated in vacuum and the residue was purified by prep-HPLC to give the title compound (12 mg, 17%). LC-MS (M+H)⁺=245.1.

### Step 3: (±)-tert-butyl ((7-(1-((2S,6S)-2-methyl-6-(5-(trifluoromethyl)pyridin-2-yl)piperidine-1-carbonyl)cyclopropyl)-4-oxo-3,4-dihydrophthalazin-1-yl)methyl)carbamate

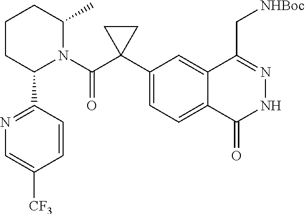 [SMILES: C[C@H]1CCC[C@@H](c2ccc(C(F)(F)F)cn2)N1C(=O)C1(c2ccc3c(=O)[nH]nc(CNC(=O)OC(C)(C)C)c3c2)CC1]

The title compound (4 mg, 14%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and (±)-2-((2S,6S)-6-methylpiperidin-2-yl)-5-(trifluoromethyl)pyridine. LC-MS (M+H)⁺=586.2.

### Step 4: (±)-4-(aminomethyl)-6-(1-((2S,6S)-2-methyl-6-(5-(trifluoromethyl)pyridin-2-yl)piperidine-1-carbonyl)cyclopropyl)phthalazin-1(2H)-one

Example 24 (0.7 mg, 21%) was prepared in a manner similar to that in Example 6 step 4 from (±)-tert-butyl ((7-(1-((2S,6S)-2-methyl-6-(5-(trifluoromethyl)pyridin-2-yl)piperidine-1-carbonyl)cyclopropyl)-4-oxo-3,4-dihydrophthalazin-1-yl)methyl)carbamate. ¹H NMR (500 MHz, DMSO-d6) δ 12.75-12.33 (m, 1H), 9.09-8.69 (m, 1H), 8.28-8.11 (m, 2H), 7.93-7.61 (m, 2H), 7.56-7.25 (m, 1H), 6.06-5.38 (m, 1H), 4.53-4.36 (m, 1H), 4.19-4.05 (m, 2H), 2.82-2.69 (m, 1H), 2.11-1.25 (m, 10H), 0.36-0.17 (m, 2H). LC-MS (M+H)⁺=486.4.

### Example 25: 1-(4-(aminomethyl)-1-oxo-1,2-dihydrophthalazin-6-yl)-N-cyclobutyl-N-((5-(2,6-difluorophenyl)pyridin-2-yl)methyl)cyclopropane-1-carboxamide

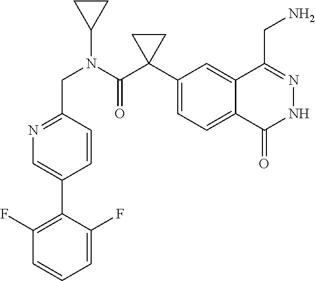 [SMILES: NCc1n[nH]c(=O)c2ccc(C3(C(=O)N(Cc4ccc(-c5c(F)cccc5F)cn4)C4CC4)CC3)cc12]

### Step 1: 5-(2,6-difluorophenyl)picolinaldehyde

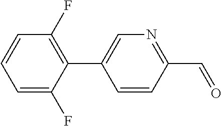 [SMILES: O=Cc1ccc(-c2c(F)cccc2F)cn1]

To a mixture of 5-(4,4,5,5-tetramethyl-1,3,2-dioxaborolan-2-yl)picolinaldehyde (20.0 g, 85.8 mmol) and 1,3-difluoro-2-iodobenzene (20.6 g, 85.8 mmol) in dioxane (100 mL), toluene (50 mL) and water (50 mL) was added Pd(dppf)Cl₂ (3.14 g, 4.29 mmol) and K₃PO₄ (18.2 g, 85.8 mmol). The mixture was stirred at 85° C. for 16 h. The mixture was cooled to room temperature and poured into water (100 mL). The organic layer was separated, and the aqueous phase was extracted with EtOAc (80 mL×2). The combined organic phase was washed with brine (50 mL), dried with anhydrous Na₂SO₄, filtered and concentrated under vacuum. The residue was purified by silica gel chromatography (PE:EtOAc=50:1 to 5:1) to give the title compound (2.6 g, 14%). LC-MS (M+H)⁺=220.2.

### Step 2: N-((5-(2,6-difluorophenyl)pyridin-2-yl)methyl)cyclobutanamine

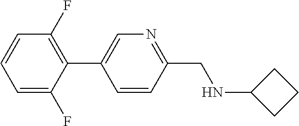 [SMILES: Fc1cccc(F)c1-c1ccc(CNC2CCC2)nc1]

The title compound (47 mg, 19%) was prepared in a manner similar to that in Example 12 step 4 from 5-(2,6-difluorophenyl)picolinaldehyde and cyclobutanamine. LC-MS (M+H)⁺=275.1.

### Step 3: tert-butyl ((7-(1-(cyclobutyl((5-(2,6-difluorophenyl)pyridin-2-yl)methyl)carbamoyl)cyclopropyl)-4-oxo-3,4-dihydrophthalazin-1-yl)methyl)carbamate

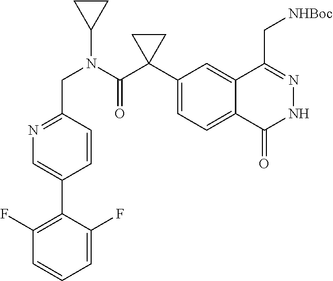 [SMILES: CC(C)(C)OC(=O)NCc1n[nH]c(=O)c2ccc(C3(C(=O)N(Cc4ccc(-c5c(F)cccc5F)cn4)C4CC4)CC3)cc12]

The title compound (15 mg, 14%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(2,6-difluorophenyl)pyridin-2-yl)methyl)cyclobutanamine. LC-MS (M+H)⁺=615.3.

### Step 4: 1-(4-(aminomethyl)-1-oxo-1,2-dihydrophthalazin-6-yl)-N-cyclobutyl-N-((5-(2,6-difluorophenyl)pyridin-2-yl)methyl)cyclopropane-1-carboxamide

Example 25 (7 mg, 56%) was prepared in a manner similar to that in Example 6 step 4 from tert-butyl ((7-(1-(cyclobutyl((5-(2,6-difluorophenyl)pyridin-2-yl)methyl)carbamoyl)cyclopropyl)-4-oxo-3,4-dihydrophthalazin-1-yl)methyl)carbamate. 1H NMR (500 MHz, DMSO-d6) δ 12.57 (s, 1H), 8.80-8.40 (m, 1H), 8.35-7.65 (m, 4H), 7.63-7.02 (m, 4H), 4.88-4.61 (m, 3H), 4.29-3.98 (m, 2H), 2.05-1.95 (m, 2H), 1.69-1.10 (m, 10H). LC-MS (M+H)⁺=516.4.

### Example 26: 1-(4-(aminomethyl)-1-oxo-1,2-dihydrophthalazin-6-yl)-N-((5-(2-chloro-6-fluorophenyl)pyridin-2-yl)methyl)-N-(5,6,7,8-tetrahydroquinolin-8-yl)cyclopropane-1-carboxamide

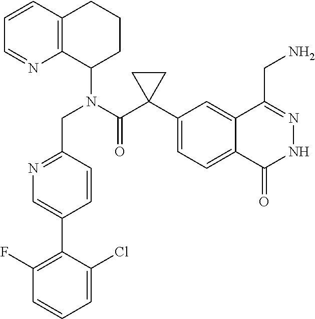 [SMILES: NCc1n[nH]c(=O)c2ccc(C3(C(=O)N(Cc4ccc(-c5c(F)cccc5Cl)cn4)C4CCCc5cccnc54)CC3)cc12]

### Step 1: tert-butyl ((5-(2-chloro-6-fluorophenyl)pyridin-2-yl)methyl)carbamate

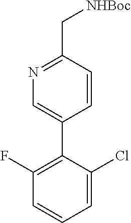 [SMILES: CC(C)(C)OC(=O)NCc1ccc(-c2c(F)cccc2Cl)cn1]

The title compound (180 mg, 69%) was prepared in a manner similar to that in Example 18 step 1 from tert-butyl ((5-bromopyridin-2-yl)methyl)carbamate and 1-chloro-3-fluoro-2-iodobenzene. LC-MS (M+H)⁺=337.2.

### Step 2: N-((5-(2-chloro-6-fluorophenyl)pyridin-2-yl)methyl)-5,6,7,8-tetrahydroquinolin-8-amine

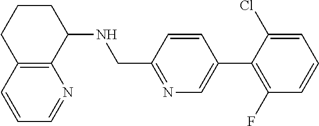 [SMILES: Fc1cccc(Cl)c1-c1ccc(CNC2CCCc3cccnc32)nc1]

The title compound (60 mg, 29%) was prepared in a manner similar to that in Example 6 step 3 from tert-butyl ((5-(2-chloro-6-fluorophenyl)pyridin-2-yl)methyl)carbamate and 6,7-dihydroquinolin-8(5H)-one. LC-MS (M+H)⁺=368.1.

### Step 3: tert-butyl ((7-(1-(((5-(2-chloro-6-fluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

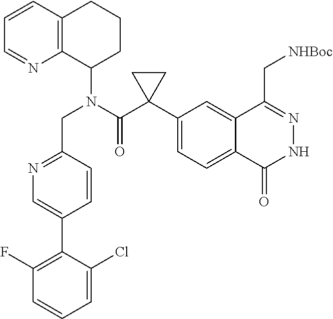 [SMILES: CC(C)(C)OC(=O)NCc1n[nH]c(=O)c2ccc(C3(C(=O)N(Cc4ccc(-c5c(F)cccc5Cl)cn4)C4CCCc5cccnc54)CC3)cc12]

The title compound (30 mg, 38%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(2-chloro-6-fluorophenyl)pyridin-2-yl)methyl)-5,6,7,8-tetrahydroquinolin-8-amine. LC-MS (M+H)⁺=709.4.

### Step 4: 1-(4-(aminomethyl)-1-oxo-1,2-dihydrophthalazin-6-yl)-N-((5-(2-chloro-6-fluorophenyl)pyridin-2-yl)methyl)-N-(5,6,7,8-tetrahydroquinolin-8-yl)cyclopropane-1-carboxamide

Example 26 (14 mg, 55%) was prepared in a manner similar to that in Example 6 step 5 from tert-butyl ((7-(1-(((5-(2-chloro-6-fluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (400 MHz, DMSO-d6) δ 12.53-12.33 (m, 1H), 8.53-8.45 (m, 1H), 8.44-8.32 (m, 1H), 8.30-8.08 (m, 1H), 8.05-8.00 (m, 1H), 7.91-7.74 (m, 2H), 7.56-7.30 (m, 5H), 7.25-7.15 (m, 1H), 5.60-5.52 (m, 1H), 4.91-4.69 (m, 1H), 4.12-3.92 (m, 2H), 3.64-3.56 (m, 1H), 2.80-2.57 (m, 2H), 1.91-1.82 (m, 1H), 1.78-1.14 (m, 6H), 1.00-0.83 (m, 1H). LC-MS (M+H)⁺=609.4.

### Example 27: 1-(4-(aminomethyl)-1-oxo-1,2-dihydrophthalazin-6-yl)-N-((5-bromoquinolin-2-yl)methyl)-N-(5,6,7,8-tetrahydroquinolin-8-yl)cyclopropane-1-carboxamide

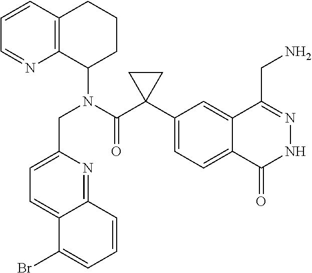 [SMILES: NCc1n[nH]c(=O)c2ccc(C3(C(=O)N(Cc4ccc5c(Br)cccc5n4)C4CCCc5cccnc54)CC3)cc12]

### Step 1: tert-butyl ((5-bromoquinolin-2-yl)methyl)carbamate

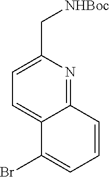 [SMILES: CC(C)(C)OC(=O)NCc1ccc2c(Br)cccc2n1]

The title compound (222 mg, 26%) was prepared in a manner similar to that in Example 7 step 2 from 5-bromoquinoline-2-carbonitrile. LC-MS (M+H)⁺=337.2.

### Step 2: N-((5-bromoquinolin-2-yl)methyl)-5,6,7,8-tetrahydroquinolin-8-amine

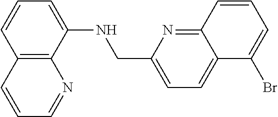 [SMILES: Brc1cccc2nc(CNc3cccc4cccnc34)ccc12]

The title compound (50 mg, 16%) was prepared in a manner similar to that in Example 6 step 3 from tert-butyl ((5-bromoquinolin-2-yl)methyl)carbamate and 6,7-dihydroquinolin-8(5H)-one. LC-MS (M+H)⁺=368.1.

### Step 3: tert-butyl ((7-(1-(((5-bromoquinolin-2-yl)methyl)(5,6,7,8-tetrahydroquinolin-8-yl)carbamoyl)cyclopropyl)-4-oxo-3,4-dihydrophthalazin-1-yl)methyl)carbamate

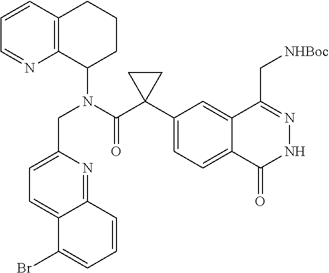 [SMILES: CC(C)(C)OC(=O)NCc1n[nH]c(=O)c2ccc(C3(C(=O)N(Cc4ccc5c(Br)cccc5n4)C4CCCc5cccnc54)CC3)cc12]

The title compound (30 mg, 51%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-bromoquinolin-2-yl)methyl)-5,6,7,8-tetrahydroquinolin-8-amine. LC-MS (M+H)⁺=709.3.

### Step 4: 1-(4-(aminomethyl)-1-oxo-1,2-dihydrophthalazin-6-yl)-N-((5-bromoquinolin-2-yl)methyl)-N-(5,6,7,8-tetrahydroquinolin-8-yl)cyclopropane-1-carboxamide

Example 27 (7 mg, 27%) was prepared in a manner similar to that in Example 6 step 5 from tert-butyl ((7-(1-(((5-bromoquinolin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (500 MHz, DMSO-d6) δ 12.55-12.2 (m, 1H), 8.50-8.40 (m, 1H), 8.36-8.30 (m, 1H), 8.18-8.13 (m, 1H), 8.05-7.87 (m, 3H), 7.84-7.66 (m, 1H), 7.64-7.45 (m, 2H), 7.28-7.14 (m, 1H), 5.65-5.56 (m, 1H), 4.95-4.88 (m, 1H), 4.08-3.83 (m, 2H), 3.82-3.72 (m, 1H), 2.75-2.60 (m, 2H), 1.97-1.83 (m, 1H), 1.76-1.20 (m, 6H), 1.01-0.92 (m, 1H). LC-MS (M+H)⁺=609.3.

### Example 28: 1-(4-(aminomethyl)-1-oxo-1,2-dihydrophthalazin-6-yl)-N-((5-(2-cyano-6-fluorophenyl)pyridin-2-yl)methyl)-N-(5,6,7,8-tetrahydroquinolin-8-yl)cyclopropane-1-carboxamide

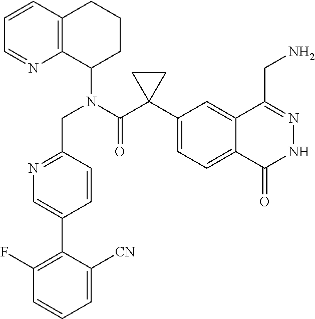 [SMILES: N#Cc1cccc(F)c1-c1ccc(CN(C(=O)C2(c3ccc4c(=O)[nH]nc(CN)c4c3)CC2)C2CCCc3cccnc32)nc1]

### Step 1: tert-butyl ((5-(2-cyano-6-fluorophenyl)pyridin-2-yl)methyl)carbamate

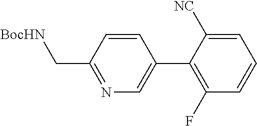 [SMILES: CC(C)(C)OC(=O)NCc1ccc(-c2c(F)cccc2C#N)cn1]

The title compound (308 mg, 77%) was prepared in a manner similar to that in Example 18 step 1 from tert-butyl ((5-bromopyridin-2-yl)methyl)carbamate and 3-fluoro-2-iodobenzonitrile. ¹H NMR (400 MHz, DMSO-d6) δ 8.65 (s, 1H), 8.00 (d, J=8.0 Hz, 1H), 7.89 (d, J=6.8 Hz, 1H), 7.82-7.73 (m, 1H), 7.73-7.66 (m, 1H), 7.56-7.50 (m, 1H), 7.44 (d, J=8.0 Hz, 1H), 4.36-4.28 (m, 2H), 4.50 (d, J=5.6 Hz, 2H), 1.40 (s, 9H).

### Step 2: 3-fluoro-2-(6-(((5,6,7,8-tetrahydroquinolin-8-yl)amino)methyl)pyridin-3-yl)benzonitrile

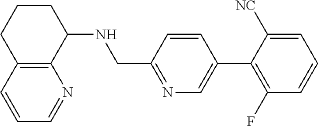 [SMILES: N#Cc1cccc(F)c1-c1ccc(CNC2CCCc3cccnc32)nc1]

The title compound (162 mg, 34%) was prepared in a manner similar to that in Example 6 step 3 from tert-butyl ((5-(2-cyano-6-fluorophenyl)pyridin-2-yl)methyl)carbamate and 6,7-dihydroquinolin-8(5H)-one. LC-MS (M+H)⁺=359.1.

### Step 3: tert-butyl ((7-(1-(((5-(2-cyano-6-fluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

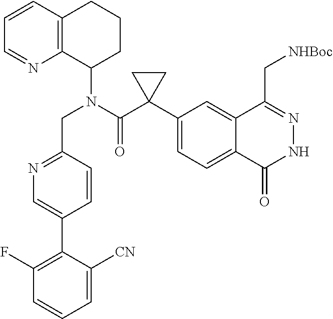 [SMILES: CC(C)(C)OC(=O)NCc1n[nH]c(=O)c2ccc(C3(C(=O)N(Cc4ccc(-c5c(F)cccc5C#N)cn4)C4CCCc5cccnc54)CC3)cc12]

The title compound (30 mg, 38%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and 3-fluoro-2-(6-(((5,6,7,8-tetrahydroquinolin-8-yl)amino)methyl)pyridin-3-yl)benzonitrile. LC-MS (M+H)⁺=700.4.

### Step 4: 1-(4-(aminomethyl)-1-oxo-1,2-dihydrophthalazin-6-yl)-N-((5-(2-cyano-6-fluorophenyl)pyridin-2-yl)methyl)-N-(5,6,7,8-tetrahydroquinolin-8-yl)cyclopropane-1-carboxamide

Example 28 (7 mg, 28%) was prepared in a manner similar to that in Example 6 step 5 from tert-butyl ((7-(1-(((5-(2-cyano-6-fluorophenyl)pyridin-2-yl)methyl)(5,6,7,8-tetrahydroquinolin-8-yl)carbamoyl)cyclopropyl)-4-oxo-3,4-dihydrophthalazin-1-yl)methyl)carbamate and TFA. ¹H NMR (500 MHz, DMSO-d6) δ 12.56-12.40 (m, 1H), 8.70-8.54 (m, 1H), 8.50-8.40 (m, 1H), 8.30-8.12 (m, 1H), 8.04-7.92 (m, 2H), 7.92-7.83 (m, 2H), 7.81-7.75 (m, 1H), 7.73-7.66 (m, 1H), 7.56-7.46 (m, 1H), 7.39 (d, J=8.2 Hz, 1H), 7.26-7.16 (m, 1H), 5.62-5.52 (m, 1H), 4.88-4.66 (m, 1H), 4.18-3.96 (m, 2H), 3.68-3.60 (m, 1H), 2.78-2.58 (m, 2H), 1.90-1.83 (m, 1H), 1.76-1.54 (m, 4H), 1.51-1.37 (m, 1H), 1.34-1.23 (m, 1H), 0.95-0.88 (m, 1H). LC-MS (M+H)⁺=600.4.

### Example 29: 1-(4-(aminomethyl)-1-oxo-1,2-dihydrophthalazin-6-yl)-N-(1-(pyrimidin-2-yl)ethyl)-N-((5-(trifluoromethyl)pyridin-2-yl)methyl)cyclopropane-1-carboxamide

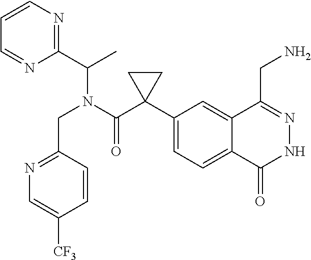 [SMILES: CC(c1ncccn1)N(Cc1ccc(C(F)(F)F)cn1)C(=O)C1(c2ccc3c(=O)[nH]nc(CN)c3c2)CC1]

### Step 1: 1-(pyrimidin-2-yl)-N-((5-(trifluoromethyl)pyridin-2-yl)methyl)ethan-1-amine

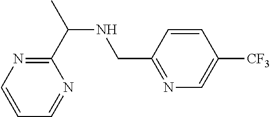 [SMILES: CC(NCc1ccc(C(F)(F)F)cn1)c1ncccn1]

The title compound (5.0 g, 31%) was prepared in a manner similar to that in Example 3 step 3 from (5-(trifluoromethyl)pyridin-2-yl)methanamine and 1-(pyrimidin-2-yl)ethan-1-one. LC-MS (M+H)⁺=283.1.

### Step 2: tert-butyl ((4-oxo-7-(1-((1-(pyrimidin-2-yl)ethyl)((5-(trifluoromethyl)pyridin-2-yl)methyl)carbamoyl)cyclopropyl)-3,4-dihydrophthalazin-1-yl)methyl)carbamate

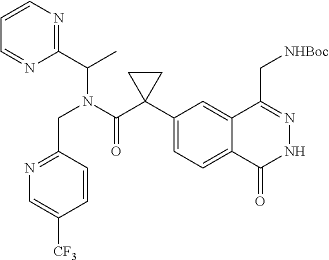 [SMILES: CC(c1ncccn1)N(Cc1ccc(C(F)(F)F)cn1)C(=O)C1(c2ccc3c(=O)[nH]nc(CNC(=O)OC(C)(C)C)c3c2)CC1]

The title compound (110 mg, 32%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and 1-(pyrimidin-2-yl)-N-((5-(trifluoromethyl)pyridin-2-yl)methyl)ethan-1-amine. LC-MS (M+Na)⁺=646.2.

### Step 3: 1-(4-(aminomethyl)-1-oxo-1,2-dihydrophthalazin-6-yl)-N-(1-(pyrimidin-2-yl)ethyl)-N-((5-(trifluoromethyl)pyridin-2-yl)methyl)cyclopropane-1-carboxamide

Example 29 (31 mg, 37%) was prepared in a manner similar to that in Example 6 step 4 from tert-butyl ((4-oxo-7-(1-((1-(pyrimidin-2-yl)ethyl)((5-(trifluoromethyl)pyridin-2-yl)methyl)carbamoyl)cyclopropyl)-3,4-dihydrophthalazin-1-yl)methyl)carbamate. ¹H NMR (400 MHz, DMSO-d6) δ 12.89-12.76 (m, 1H), 8.93-8.81 (m, 1H), 8.75-8.58 (m, 3H), 8.29-8.19 (m, 1H), 8.05-7.91 (m, 1H), 7.81-7.21 (m, 3H), 5.75-5.16 (m, 1H), 5.12-4.88 (m, 1H), 4.87-4.26 (m, 3H), 1.70-1.12 (m, 7H). LC-MS (M+H)⁺=524.2.

### Example 30: 1-(4-(aminomethyl)-1-oxo-1,2-dihydrophthalazin-6-yl)-N-((5-(2,6-difluorophenyl)pyridin-2-yl)methyl)-N-(1,3-dimethoxypropan-2-yl)cyclopropane-1-carboxamide

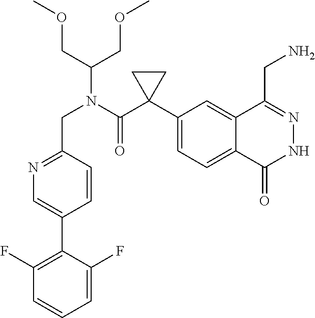 [SMILES: COCC(COC)N(Cc1ccc(-c2c(F)cccc2F)cn1)C(=O)C1(c2ccc3c(=O)[nH]nc(CN)c3c2)CC1]

### Step 1: N-((5-(2,6-difluorophenyl)pyridin-2-yl)methyl)-1,3-dimethoxypropan-2-amine

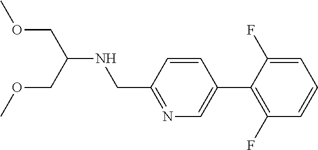 [SMILES: COCC(COC)NCc1ccc(-c2c(F)cccc2F)cn1]

The title compound (0.20 g, 76%) was prepared in a manner similar to that in Example 3 step 3 from 1,3-dimethoxypropan-2-one and (5-(2,6-difluorophenyl)pyridin-2-yl)methanamine. ¹H NMR (400 MHz, DMSO-d6) δ 8.57 (s, 1H), 7.87 (d, J=8.4 Hz, 1H), 7.60-7.48 (m, 2H), 7.32-7.22 (m, 2H), 3.94 (s, 2H), 3.34 (d, J=5.6 Hz, 4H), 3.24 (s, 6H), 2.89 (quin, J=5.6 Hz, 1H). LC-MS (M+H)⁺=323.2.

### Step 2: tert-butyl ((7-(1-(((5-(2,6-difluorophenyl)pyridin-2-yl)methyl)(1,3-dimethoxypropan-2-yl)carbamoyl)cyclopropyl)-4-oxo-3,4-dihydrophthalazin-1-yl)methyl)carbamate

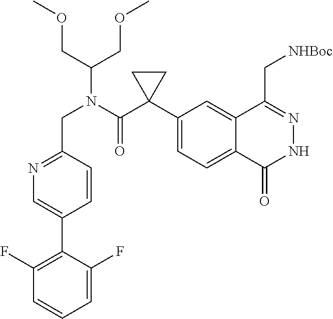 [SMILES: COCC(COC)N(Cc1ccc(-c2c(F)cccc2F)cn1)C(=O)C1(c2ccc3c(=O)[nH]nc(CNC(=O)OC(C)(C)C)c3c2)CC1]

The title compound (40 mg, 15%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(2,6-difluorophenyl)pyridin-2-yl)methyl)-1,3-dimethoxypropan-2-amine. LC-MS (M+H)⁺=664.3.

### Step 3: 1-(4-(aminomethyl)-1-oxo-1,2-dihydrophthalazin-6-yl)-N-((5-(2,6-difluorophenyl)pyridin-2-yl)methyl)-N-(1,3-dimethoxypropan-2-yl)cyclopropane-1-carboxamide

Example 30 (7 mg, 21%) was prepared in a manner similar to that in Example 6 step 5 from tert-butyl ((7-(1-(((5-(2,6-difluorophenyl)pyridin-2-yl)methyl)(1,3-dimethoxypropan-2-yl)carbamoyl)cyclopropyl)-4-oxo-3,4-dihydrophthalazin-1-yl)methyl)carbamate. ¹H NMR (400 MHz, CD₃OD) δ 8.70-8.40 (m, 1H), 8.38-8.22 (m, 1H), 7.92-7.86 (m, 2H), 7.76-7.42 (m, 3H), 7.20-7.05 (m, 2H), 4.80-4.33 (m, 3H), 4.20-4.08 (m, 2H), 3.81-3.52 (m, 1H), 3.30-3.18 (m, 3H), 2.97 (s, 6H), 1.73-1.67 (m, 2H), 1.51-1.39 (m, 2H). LC-MS (M+H)⁺=564.2.

### Example 31: (R)-1-(4-(aminomethyl)-1-oxo-1,2-dihydrophthalazin-6-yl)-N-(4-(pentafluoro-λ⁶-sulfanyl)benzyl)-N-(5,6,7,8-tetrahydroquinolin-8-yl)cyclopropane-1-carboxamide

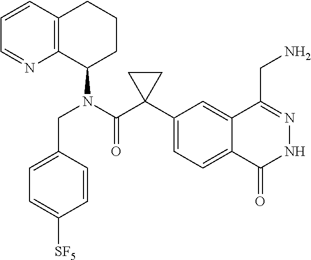

### Step 1: (R)—N-(4-(pentafluoro-λ⁶-sulfanyl)benzyl)-5,6,7,8-tetrahydroquinolin-8-amine

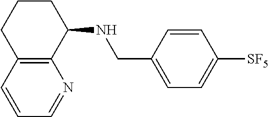

The title compound (0.20 g, 55%) was prepared in a manner similar to that in Example 12 step 4 from 4-(pentafluoro-λ⁶-sulfanyl)benzaldehyde and (R)-5,6,7,8-tetrahydroquinolin-8-amine. ¹H NMR (400 MHz, DMSO-d6) δ 8.36 (d, J=4.4 Hz, 1H), 7.84 (d, J=8.8 Hz, 2H), 7.62 (d, J=8.8 Hz, 2H), 7.50 (d, J=7.6 Hz, 1H), 7.18 (dd, J=7.6, 4.4 Hz, 1H), 4.00-3.88 (m, 2H), 3.72-3.63 (m, 1H), 3.13-2.98 (m, 1H), 2.82-2.68 (m, 2H), 1.72-1.60 (m, 2H). LCMS (M+H)⁺=365.1.

### Step 2: tert-butyl (R)-((4-oxo-7-(1-((4-(pentafluoro-λ⁶-sulfanyl)benzyl)(5,6,7,8-tetrahydroquinolin-8-yl)carbamoyl)cyclopropyl)-3,4-dihydrophthalazin-1-yl)methyl)carbamate

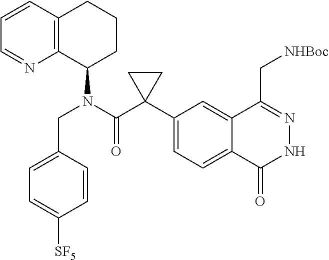

The title compound (0.20 g, 57%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and (R)—N-(4-(pentafluoro-λ⁶-sulfanyl)benzyl)-5,6,7,8-tetrahydroquinolin-8-amine. LCMS (M+H)⁺=706.2.

### Step 3: (R)-1-(4-(aminomethyl)-1-oxo-1,2-dihydrophthalazin-6-yl)-N-(4-(pentafluoro-λ⁶-sulfanyl)benzyl)-N-(5,6,7,8-tetrahydroquinolin-8-yl)cyclopropane-1-carboxamide

Example 31 (57 mg, 34%) was prepared in a manner similar to that in Example 6 step 5 from tert-butyl (R)-((4-oxo-7-(1-((4-(pentafluoro-λ⁶-sulfanyl)benzyl)(5,6,7,8-tetrahydroquinolin-8-yl)carbamoyl)cyclopropyl)-3,4-dihydrophthalazin-1-yl)methyl)carbamate. ¹H NMR (400 MHz, CD₃OD) δ 8.47-8.18 (m, 2H), 7.96-7.66 (m, 3H), 7.51-7.12 (m, 4H), 5.81-4.73 (m, 2H), 4.20-3.63 (m, 3H), 2.96-2.68 (m, 2H), 2.24-1.95 (m, 1H), 1.86-1.09 (m, 7H). LC-MS (M+H)⁺=606.1.

### Example 32: 1-(4-(aminomethyl)-1-oxo-1,2-dihydrophthalazin-6-yl)-N-(4-cyclopropyl-5,6,7,8-tetrahydroquinolin-8-yl)-N-((5-(trifluoromethyl)pyridin-2-yl)methyl)cyclopropane-1-carboxamide

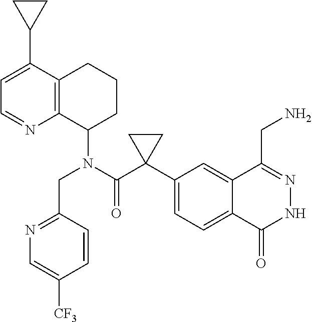 [SMILES: NCc1n[nH]c(=O)c2ccc(C3(C(=O)N(Cc4ccc(C(F)(F)F)cn4)C4CCCc5c(C6CC6)ccnc54)CC3)cc12]

### Step 1: 4-methoxy-5,6,7,8-tetrahydroquinoline

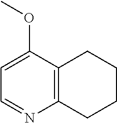

To a solution of PtO₂ (3.71 g, 16.3 mmol) in TFA (200 mL) was added 4-methoxyquinoline (20 g, 126 mmol) under nitrogen. The suspension was degassed and purged with hydrogen for 3 times. The mixture was stirred under hydrogen (50 psi) at 50° C. for 3 h. The filtrate was collected by filtration, concentration to about 50 mL and diluted with water (100 mL). The pH of the mixture was adjusted to 9 with aqueous NaOH (1 M). The mixture was extracted with DCM (50 mL×3). The combined organic layer was washed with brine (50 mL×2), dried over Na₂SO₄, filtered and concentrated under reduced pressure to give the title compound (20 g, 98%). ¹H NMR (400 MHz, CD₃OD) δ 8.40 (d, J=6.4 Hz, 1H), 7.30 (d, J=6.4 Hz, 1H), 4.11 (s, 3H), 2.96 (t, J=6.2 Hz, 2H), 2.71 (t, J=6.2 Hz, 2H), 1.96-1.83 (m, 4H). LCMS (M+H)⁺=164.2.

### Step 2: 4-bromo-5,6,7,8-tetrahydroquinoline

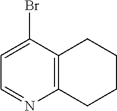 [SMILES: Brc1ccnc2c1CCCC2]

To a solution of 4-methoxy-5,6,7,8-tetrahydroquinoline (17 g, 104 mmol) in DMF (170 mL) at 0° C. was added POBr₃ (89.6 g, 312 mmol). The mixture was heated to 100° C. and stirred for 12 h. The reaction mixture was cooled to 0° C. and slowly added to iced water (300 mL). The mixture was extracted with EtOAc (100 mL×3). The combined organic layer was washed with brine (50 mL×2), dried over Na₂SO₄, filtered and concentrated under reduced pressure. The residue was purified by silica gel chromatograph (PE:EtOAc=1:0 to 5:1) to give the title compound (15 g). LCMS (M+H)⁺=212.1.

### Step 3: 4-bromo-5,6,7,8-tetrahydroquinoline 1-oxide

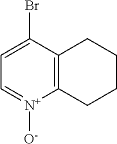 [SMILES: [O-][n+]1ccc(Br)c2c1CCCC2]

To a solution of 4-bromo-5,6,7,8-tetrahydroquinoline (15 g, 70.7 mmol) in DCM (195 mL) was added mCPBA (80%, 30.5 g, 141 mmol). The mixture was stirred at 42° C. for 2 h then cooled to room temperature. The mixture was washed with water (100 mL) and the aqueous phase was extracted with DCM (50 mL×3). The combined organic layer was washed with brine, (50 mL×2), dried over Na₂SO₄, filtered and concentrated under reduced pressure. The residue was purified by silica gel chromatograph (PE:EtOAc=1:0 to 3:1) to give the title compound (14 g, 87%). LCMS (M+H)⁺=228.2.

### Step 4: 4-bromo-5,6,7,8-tetrahydroquinolin-8-yl acetate

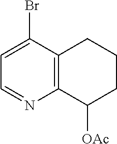 [SMILES: CC(=O)OC1CCCc2c(Br)ccnc21]

A mixture of 4-bromo-5,6,7,8-tetrahydroquinoline 1-oxide (14 g, 61.4 mmol) in Ac₂O (70 mL) was stirred at 55° C. for 2 h and cooled to room temperature. The filtrate was collected by filtration and concentrated under reduced pressure to give the title compound (14 g, 84%). LCMS (M+H)⁺=270.1.

### Step 5: 4-bromo-5,6,7,8-tetrahydroquinolin-8-ol

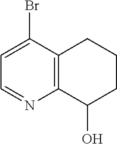 [SMILES: OC1CCCc2c(Br)ccnc21]

Four identical reactions as described below were set up in parallel. To a solution of 4-bromo-5,6,7,8-tetrahydroquinolin-8-yl acetate (3.0 g, 11.1 mmol) in MeOH (15 mL) was added K₂CO₃ (5.8 g, 42.2 mmol). The mixture was stirred at 25° C. for 2 h.

The four batches of reaction mixture were combined. The filtrate was collected by filtration and concentrated under reduced pressure. The residue was purified by silica gel chromatograph (PE:EtOAc=1:0 to 1:1) to give the title compound (5.2 g, 51%). LCMS (M+H)⁺=228.2.

### Step 6: 4-bromo-6,7-dihydroquinolin-8(5H)-one

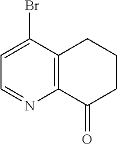 [SMILES: O=C1CCCc2c(Br)ccnc21]

To a solution of 4-bromo-5,6,7,8-tetrahydroquinolin-8-ol (5.0 g, 21.9 mmol) in DCM (260 mL) was added DMP (16.7 g, 39.5 mmol). The mixture was stirred at 25° C. for 8 h. The mixture was filtered and the filtrate was concentrated under reduced pressure. The residue was purified by silica gel chromatograph (PE:EtOAc=1:0 to 1:1) to give the title compound (4.0 g, 81%). LCMS (M+H)⁺=226.1.

### Step 7: 4-cyclopropyl-6,7-dihydroquinolin-8(5H)-one

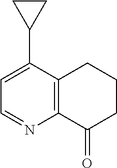 [SMILES: O=C1CCCc2c(C3CC3)ccnc21]

To a solution of 4-bromo-6,7-dihydroquinolin-8(5H)-one (1.0 g, 4.42 mmol) and cyclopropylboronic acid (950 mg, 11.1 mmol) in water (1 mL) and toluene (9 mL) was added SPhos (145 mg, 0.35 mmol), Pd(OAc)₂ (99 mg, 0.44 mmol) and K₃PO₄ (2.35 g, 11.1 mmol). The mixture was stirred at 90° C. for 12 h and cooled to room temperature. The mixture was diluted with water (10 mL) and extracted with EtOAc (15 mL×3). The combined organic layer was washed with brine (15 mL×2), dried over Na₂SO₄, filtered and concentrated under reduced pressure. The residue was purified by silica gel chromatograph (PE:EtOAc=1:0 to 1:1) to give the title compound (0.20 g, 24%). LCMS (M+H)⁺=188.3.

### Step 8: 4-cyclopropyl-N-((5-(trifluoromethyl)pyridin-2-yl)methyl)-5,6,7,8-tetrahydroquinolin-8-amine

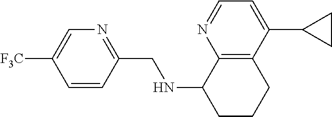 [SMILES: FC(F)(F)c1ccc(CNC2CCCc3c(C4CC4)ccnc32)nc1]

The title compound (0.20 g, 54%) was prepared in a manner similar to that in Example 3 step 3 from 4-cyclopropyl-6,7-dihydroquinolin-8(5H)-one and (5-(trifluoromethyl)pyridin-2-yl)methanamine. LCMS (M+H)⁺=348.2.

### Step 9: tert-butyl ((7-(1-((4-cyclopropyl-5,6,7,8-tetrahydroquinolin-8-yl)((5-(trifluoromethyl)pyridin-2-yl)methyl)carbamoyl)cyclopropyl)-4-oxo-3,4-dihydrophthalazin-1-yl)methyl)carbamate

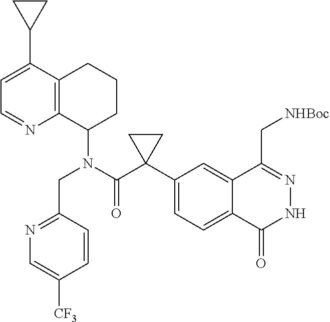 [SMILES: CC(C)(C)OC(=O)NCc1n[nH]c(=O)c2ccc(C3(C(=O)N(Cc4ccc(C(F)(F)F)cn4)C4CCCc5c(C6CC6)ccnc54)CC3)cc12]

The title compound (0.11 g, 50%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and 4-cyclopropyl-N-((5-(trifluoromethyl)pyridin-2-yl)methyl)-5,6,7,8-tetrahydroquinolin-8-amine. LCMS (M+H)⁺=689.3.

### Step 10: 1-(4-(aminomethyl)-1-oxo-1,2-dihydrophthalazin-6-yl)-N-(4-cyclopropyl-5,6,7,8-tetrahydroquinolin-8-yl)-N-((5-(trifluoromethyl)pyridin-2-yl)methyl)cyclopropane-1-carboxamide

Example 32 (57 mg, 34%) was prepared in a manner similar to that in Example 6 step 5 from tert-butyl ((7-(1-((4-cyclopropyl-5,6,7,8-tetrahydroquinolin-8-yl)((5-(trifluoromethyl)pyridin-2-yl)methyl)carbamoyl)cyclopropyl)-4-oxo-3,4-dihydrophthalazin-1-yl)methyl)carbamate. ¹H NMR (400 MHz, DMSO-d6) δ 12.62-12.37 (m, 1H), 8.95-8.56 (m, 1H), 8.38-8.22 (m, 2H), 8.20-7.77 (m, 3H), 7.76-7.37 (m, 1H), 6.83-6.77 (m, 1H), 5.60-5.50 (m, 1H), 4.96-4.65 (m, 1H), 4.09-3.90 (m, 2H), 3.66-3.55 (m, 2H), 2.88-2.78 (m, 1H), 2.68-2.57 (m, 1H), 1.92-1.78 (m, 2H), 1.78-1.18 (m, 6H), 1.05-0.86 (m, 3H), 0.81-0.71 (m, 1H), 0.67-0.54 (m, 1H). LC-MS (M+H)⁺=589.3.

### Example 33: 1-(4-(aminomethyl)-1-oxo-1,2-dihydrophthalazin-6-yl)-N-((5-(2,6-difluorophenyl)pyridin-2-yl)methyl)-N-(6,7-dihydro-5H-cyclopenta[b]pyridin-7-yl)cyclopropane-1-carboxamide

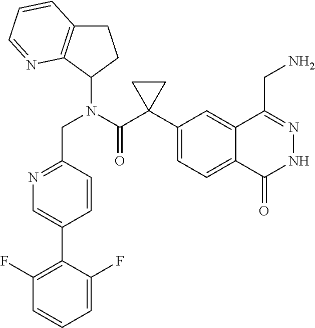 [SMILES: NCc1n[nH]c(=O)c2ccc(C3(C(=O)N(Cc4ccc(-c5c(F)cccc5F)cn4)C4CCc5cccnc54)CC3)cc12]

### Step 1: N-((5-(2,6-difluorophenyl)pyridin-2-yl)methyl)-6,7-dihydro-5H-cyclopenta[b]pyridin-7-amine

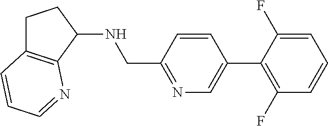 [SMILES: Fc1cccc(F)c1-c1ccc(CNC2CCc3cccnc32)nc1]

To a solution of 5-(2,6-difluorophenyl)picolinaldehyde (80 mg, 0.36 mmol) in DCM (10 mL), was added 6,7-dihydro-5H-cyclopenta[b]pyridin-7-amine (49 mg, 0.36 mmol) and NaBH(OAc)₃ (155 mg, 0.73 mmol). The solution was stirred for 16 h at room temperature, poured into saturated NaHCO₃ (50 mL) and then extracted with EtOAc (50 mL). The combined organic layer was washed with brine (50 mL), dried over Na₂SO₄, filtered and concentrated under vacuum. The residue was purified by silica gel chromatograph (DCM:MeOH=10:1) to give the title compound (31 mg, 26%). LC-MS (M+H)⁺=338.3.

### Step 2: tert-butyl ((7-(1-(((5-(2,6-difluorophenyl)pyridin-2-yl)methyl)(6,7-dihydro-5H-cyclopenta[b]pyridin-7-yl)carbamoyl)cyclopropyl)-4-oxo-3,4-dihydrophthalazin-1-yl)methyl)carbamate

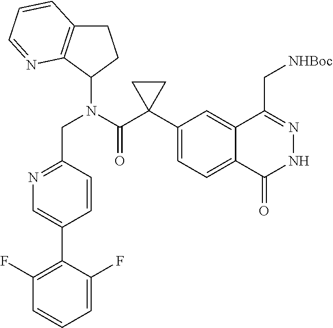 [SMILES: CC(C)(C)OC(=O)NCc1n[nH]c(=O)c2ccc(C3(C(=O)N(Cc4ccc(-c5c(F)cccc5F)cn4)C4CCc5cccnc54)CC3)cc12]

The title compound (20 mg, 36%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and N-((5-(2,6-difluorophenyl)pyridin-2-yl)methyl)-6,7-dihydro-5H-cyclopenta[b]pyridin-7-amine. LC-MS (M+H)⁺=679.1.

### Step 3: 1-(4-(aminomethyl)-1-oxo-1,2-dihydrophthalazin-6-yl)-N-((5-(2,6-difluorophenyl)pyridin-2-yl)methyl)-N-(6,7-dihydro-5H-cyclopenta[b]pyridin-7-yl)cyclopropane-1-carboxamide

Example 33 (10 mg, 59%) was prepared in a manner similar to that in Example 6 step 5 from tert-butyl ((7-(1-(((5-(2,6-difluorophenyl)pyridin-2-yl)methyl)(6,7-dihydro-5H-cyclopenta[b]pyridin-7-yl)carbamoyl)cyclopropyl)-4-oxo-3,4-dihydrophthalazin-1-yl)methyl)carbamate. ¹H NMR (500 MHz, DMSO-d6) δ 12.68-12.28 (m, 1H), 8.68-8.34 (m, 2H), 8.27-8.08 (m, 1H), 8.05-7.92 (m, 1H), 7.92-7.47 (m, 4H), 7.47-7.14 (m, 4H), 6.02-5.73 (m, 1H), 4.84-4.65 (m, 1H), 4.33-4.09 (m, 2H), 3.92-3.78 (m, 1H), 2.95-2.57 (m, 2H), 2.30-1.50 (m, 5H), 1.31-1.14 (m, 1H). LC-MS (M+H)⁺=579.4.

### Example 34: (R)-1-(4-(aminomethyl)-1-oxo-1,2-dihydrophthalazin-6-yl)-N-((5-(2,6-difluorophenoxy)pyridin-2-yl)methyl)-N-(5,6,7,8-tetrahydroquinolin-8-yl)cyclopropane-1-carboxamide

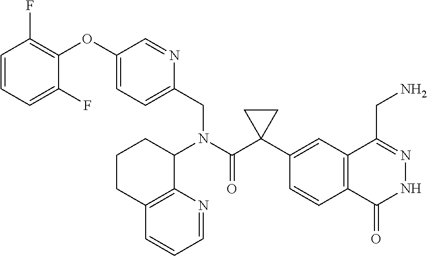 [SMILES: NCc1n[nH]c(=O)c2ccc(C3(C(=O)N(Cc4ccc(Oc5c(F)cccc5F)cn4)C4CCCc5cccnc54)CC3)cc12]

### Step 1: 5-(2,6-difluorophenoxy)picolinaldehyde

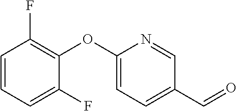 [SMILES: O=Cc1ccc(Oc2c(F)cccc2F)nc1]

The title compound (0.60 g, 64%) was prepared in a manner similar to that in Example 20 step 1 from 5-fluoropicolinaldehyde and 2,6-difluorophenol. LCMS (M+H)⁺=236.2.

### Step 2: (R)—N-((5-(2,6-difluorophenoxy)pyridin-2-yl)methyl)-5,6,7,8-tetrahydroquinolin-8-amine

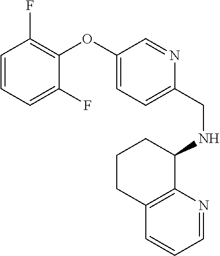 [SMILES: Fc1cccc(F)c1Oc1ccc(CN[C@@H]2CCCc3cccnc32)nc1]

The title compound (0.13 g, 42%) was prepared in a manner similar to that in Example 12 step 4 from (R)-5,6,7,8-tetrahydroquinolin-8-amine and 5-(2,6-difluorophenoxy)picolinaldehyde. LCMS (M+H)⁺=368.2.

### Step 3: tert-butyl (R)-((7-(1-(((5-(2,6-difluorophenoxy)pyridin-2-yl)methyl)(5,6,7,8-tetrahydroquinolin-8-yl)carbamoyl)cyclopropyl)-4-oxo-3,4-dihydrophthalazin-1-yl)methyl)carbamate

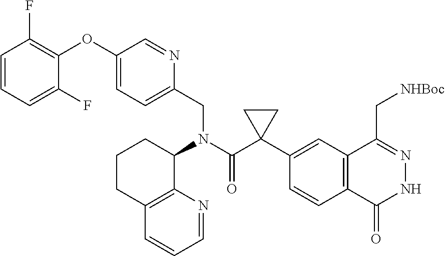 [SMILES: CC(C)(C)OC(=O)NCc1n[nH]c(=O)c2ccc(C3(C(=O)N(Cc4ccc(Oc5c(F)cccc5F)cn4)[C@@H]4CCCc5cccnc54)CC3)cc12]

The title compound (0.10 g, 74%) was prepared in a manner similar to that in Example 6 step 4 from 1-(4-(((tert-butoxycarbonyl)amino)methyl)-1-oxo-1,2-dihydrophthalazin-6-yl)cyclopropane-1-carboxylic acid and (R)—N-((5-(2,6-difluorophenoxy)pyridin-2-yl)methyl)-5,6,7,8-tetrahydroquinolin-8-amine. LCMS (M+H)⁺=709.4.

### Step 4: (R)-1-(4-(aminomethyl)-1-oxo-1,2-dihydrophthalazin-6-yl)-N-((5-(2,6-difluorophenoxy)pyridin-2-yl)methyl)-N-(5,6,7,8-tetrahydroquinolin-8-yl)cyclopropane-1-carboxamide

Example 34 (25 mg, 29%) was prepared in a manner similar to that in Example 6 step 4 from tert-butyl (R)-((7-(1-(((5-(2,6-difluorophenoxy)pyridin-2-yl)methyl)(5,6,7,8-tetrahydroquinolin-8-yl)carbamoyl)cyclopropyl)-4-oxo-3,4-dihydrophthalazin-1-yl)methyl)carbamate. ¹H NMR (400 MHz, DMSO-d6) δ 12.60-12.38 (m, 1H), 8.46-8.39 (m, 1H), 8.30-8.10 (m, 2H), 7.97-7.71 (m, 2H), 7.53-7.04 (m, 7H), 5.55-5.45 (m, 1H), 4.72-4.62 (m, 1H), 4.96-4.65 (m, 1H), 4.50-3.94 (m, 2H), 3.53-3.43 (m, 1H), 2.67-2.56 (m, 2H), 1.90-1.30 (m, 6H), 1.23-1.08 (m, 1H), 0.96-0.82 (m, 1H). LC-MS (M+H)⁺=609.2.

### Example 35: 1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclobutane-1-carboxamide

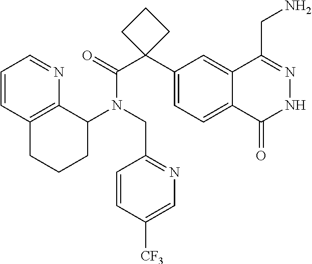 [SMILES: NCc1n[nH]c(=O)c2ccc(C3(C(=O)N(Cc4ccc(C(F)(F)F)cn4)C4CCCc5cccnc54)CCC3)cc12]

### Step 1: 5-(4,4,5,5-tetramethyl-1,3,2-dioxaborolan-2-yl)isobenzofuran-1(3H)-one

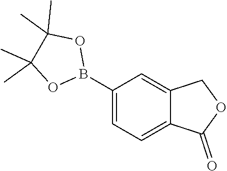 [SMILES: CC1(C)OB(c2ccc3c(c2)COC3=O)OC1(C)C]

A mixture of 5-bromoisobenzofuran-1(3H)-one (8.52 g, 40 mmol), BPD (13.2 g, 52.0 mmol), Pd(dppf)Cl₂CH₂Cl₂ (1.64 g, 2.0 mmol) and AcOK (11.8 g, 120 mmol) in dioxane (160 mL) was heated to 100° C. and stirred for overnight under nitrogen. The mixture was cooled to room temperature and solid was filtered off, then rinsed with DCM (400 mL). The filtrate was concentrated and the residue was purified by silica gel chromatograph (PE/EtOAc=15/1) to give the title compound (8.63 g, 83%). LC-MS (M+H)⁺=261.2.

### Step 2: tert-butyl 2-(1-oxo-1,3-dihydroisobenzofuran-5-yl)acetate

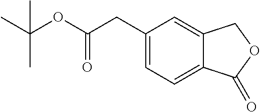 [SMILES: CC(C)(C)OC(=O)Cc1ccc2c(c1)COC2=O]

A mixture of 5-(4,4,5,5-tetramethyl-1,3,2-dioxaborolan-2-yl)isobenzofuran-1(3H)-one (8.37 g, 32.2 mmol), [Pd(allyl)Cl]₂ (590 mg, 1.61 mmol), XPhos (3.06 g, 6.44 mmol) and K₂CO₃ (11.1 g, 80.5 mmol) in dioxane (80 mL) and water (8 mL) was stirred at 90° C. for 4 h. The mixture was cooled to room temperature and diluted with water (100 mL) and the mixture was extracted with EtOAc (100 mL×3). The combined organic layer was concentrated and the residue was purified by silica gel chromatograph (PE/EtOAc=10/1) to give the title compound (4.5 g, 56%). LCMS (M+H)⁺=249.2.

### Step 3: tert-butyl 1-(1-oxo-1,3-dihydroisobenzofuran-5-yl)cyclobutane-1-carboxylate

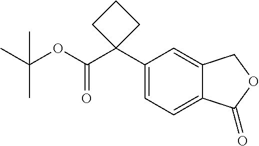 [SMILES: CC(C)(C)OC(=O)C1(c2ccc3c(c2)COC3=O)CCC1]

To a solution of tert-butyl 2-(1-oxo-1,3-dihydroisobenzofuran-5-yl)acetate (3.05 g, 12.3 mmol) in DMF (60 mL) was added Cs₂CO₃ (10 g, 30.8 mmol) and the mixture was stirred at 0° C. for 0.5 h under nitrogen. A solution of 1,3-dibromopropane (2.73 g, 13.5 mmol) in DMF (10 mL) was added dropwise, and the mixture was warmed to room temperature and stirred for overnight. Saturated NH₄Cl (200 mL) was added, and the mixture was extracted with EtOAc (80 mL×3). The combined organic layer was dried over Na₂SO₄, filtered and concentrated under reduced pressure. The residue was purified by silica gel chromatograph (PE/EtOAc=10/1) to give the title compound (1.6 g, 45%). LCMS (M+H)⁺=289.2.

### Step 4: tert-butyl 1-(3-((dimethylamino)methylene)-1-oxo-1,3-dihydroisobenzofuran-5-yl)cyclobutane-1-carboxylate

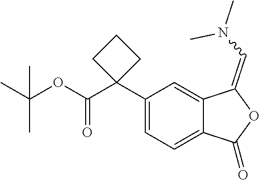 [SMILES: CN(C)C=C1OC(=O)c2ccc(C3(C(=O)OC(C)(C)C)CCC3)cc21]

A mixture of tert-butyl 1-(1-oxo-1,3-dihydroisobenzofuran-5-yl)cyclobutane-1-carboxylate (1.4 g, 4.86 mmol), DMF-DMA (10 mL) and t-BuOK (54 mg, 0.486 mmol) was degassed and purged with nitrogen 3 times and then stirred at 120° C. for 20 h. The mixture was cooled to room temperature and concentrated under reduced pressure. The residue was purified by neutral alumina chromatograph (PE/EtOAc=10/1) to give the title compound (656 mg, 39%). LCMS (M+H)⁺=344.2.

### Step 5: tert-butyl 1-(4-((dimethylamino)methyl)-1-oxo-1,2-dihydrophthalazin-6-yl)cyclobutane-1-carboxylate

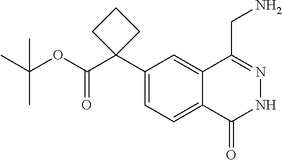 [SMILES: CC(C)(C)OC(=O)C1(c2ccc3c(=O)[nH]nc(CN)c3c2)CCC1]

To a mixture of tert-butyl 1-(4-((dimethylamino)methyl)-1-oxo-1,2-dihydrophthalazin-6-yl)cyclobutane-1-carboxylate (656 mg, 1.9 mmol) in EtOH (10 mL) was added hydrazine monohydrate (237 mg, 3.8 mmol) at 25° C. The mixture was degassed with nitrogen then stirred at 70° C. for 12 h. The mixture was cooled to room temperature and concentrated under vacuum. The residue was purified by silica gel chromatograph (PE/EtOAc=1/1) to give the title compound (500 mg, 74%). LCMS (M+H)⁺=358.3.

### Step 6: tert-butyl 1-(4-(chloromethyl)-1-oxo-1,2-dihydrophthalazin-6-yl)cyclobutane-1-carboxylate

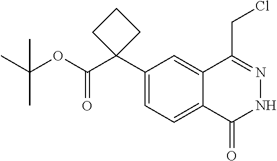 [SMILES: CC(C)(C)OC(=O)C1(c2ccc3c(=O)[nH]nc(CCl)c3c2)CCC1]

A solution of tert-butyl 1-(4-((dimethylamino)methyl)-1-oxo-1,2-dihydrophthalazin-6-yl)cyclobutane-1-carboxylate (400 mg, 1.4 mmol) in THF (10 mL) was degassed with nitrogen for 3 times and cooled to 0° C. Isobutyl chloroformate (286 mg, 2.1 mmol) in THF (2 mL) was added dropwise. The mixture was stirred at 25° C. for 6 h then concentrated under vacuum to give the title compound (240 mg, 60%). LCMS (M+H)⁺=349.2.

### Step 7: tert-butyl 1-(4-((1,3-dioxoisoindolin-2-yl)methyl)-1-oxo-1,2-dihydrophthalazin-6-yl)cyclobutane-1-carboxylate

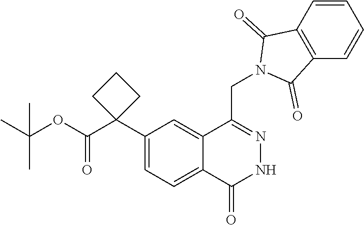 [SMILES: CC(C)(C)OC(=O)C1(c2ccc3c(=O)[nH]nc(CN4C(=O)c5ccccc5C4=O)c3c2)CCC1]

To a mixture of tert-butyl 1-(4-(chloromethyl)-1-oxo-1,2-dihydrophthalazin-6-yl)cyclobutane-1-carboxylate (240 mg, 0.84 mmol) in DMF (10 mL) was added potassium phthalate (233 mg, 1.26 mmol) and stirred at 25° C. for 1 h. The mixture was diluted with water (100 mL) and extracted with EtOAc (50 mL×3). The combined organic layer was dried over Na₂SO₄, filtered, and concentrated under reduced pressure. The residue was purified by silica gel chromatograph (PE/EtOAc=2/1) to give the title compound (220 mg, 57%). LCMS (M+H)⁺=460.3.

### Step 8: 1-(4-((1,3-dioxoisoindolin-2-yl)methyl)-1-oxo-1,2-dihydrophthalazin-6-yl)cyclobutane-1-carboxylic acid

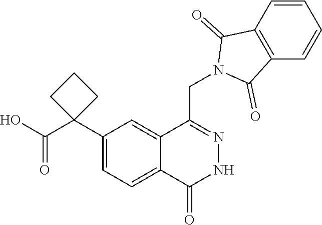 [SMILES: O=C1c2ccccc2C(=O)N1Cc1n[nH]c(=O)c2ccc(C3(C(=O)O)CCC3)cc12]

A solution of tert-butyl 1-(4-((1,3-dioxoisoindolin-2-yl)methyl)-1-oxo-1,2-dihydrophthalazin-6-yl)cyclobutane-1-carboxylate (120 mg, 0.26 mmol) in DCM (4 mL) and TFA (2 mL) was stirred at room temperature for 8 h. The mixture was concentrated under reduced pressure to give the title compound (100 mg, 95%). LCMS (M+H)⁺=404.2.

### Step 9: 1-(4-((1,3-dioxoisoindolin-2-yl)methyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclobutane-1-carboxamide

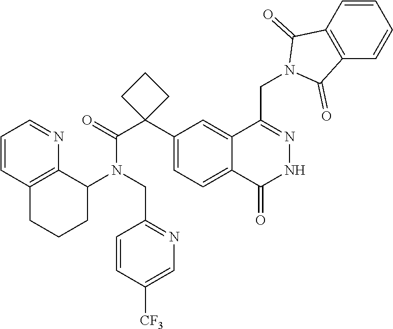 [SMILES: O=C1c2ccccc2C(=O)N1Cc1n[nH]c(=O)c2ccc(C3(C(=O)N(Cc4ccc(C(F)(F)F)cn4)C4CCCc5cccnc54)CCC3)cc12]

A solution of 1-(4-((1,3-dioxoisoindolin-2-yl)methyl)-1-oxo-1,2-dihydrophthalazin-6-yl)cyclobutane-1-carboxylic acid (100 mg, 0.25 mmol) in SOCl₂ (3 mL) was stirred at room temperature for 2 h. The mixture was concentrated under reduced pressure. The residue was dissolved in DCM (5 mL) and the solution was added dropwise to a mixture of Et₃N (242 mg, 2.4 mmol) and N-((5-(trifluoromethyl)pyridin-2-yl)methyl)-5,6,7,8-tetrahydroquinolin-8-amine (74 mg, 0.24 mmol) in DCM (10 mL) at 0° C. After 15 min, the mixture was concentrated under reduced pressure. The residue was purified by prep-TLC to give the title compound (120 mg, 69%). LCMS (M+H)⁺=693.4.

### Step 10: 1-(4-(aminomethyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclobutane-1-carboxamide

A mixture of 1-(4-((1,3-dioxoisoindolin-2-yl)methyl)-1-oxo-1,2-dihydrophthalazin-6-yl)-N-(5,6,7,8-tetrahydroquinolin-8-yl)-N-((5-(trifluoromethyl)pyridin-2-yl)methyl)cyclobutane-1-carboxamide (120 mg, 0.17 mmol) and hydrazine monohydrate (80%, 32 mg, 0.51 mmol) in EtOH (4 mL) was stirred at 80° C. for 3 h. The mixture was concentrated under reduced pressure. The residue was purified by Pre-HPLC to give Example 35 (20 mg, 20%). 1H NMR (500 MHz, DMSO) δ=12.45 (s, 1H), 8.79-8.56 (m, 1H), 8.38-8.36 (m, 2H), 8.15-7.65 (m, 3H), 7.55-7.47 (m, 1H), 7.31-7.18 (m, 2H), 4.82-4.51 (m, 2H), 4.10-3.90 (m, 2H), 3.78-3.58 (m, 1H), 3.22-3.00 (m, 3H), 2.64-2.56 (m, 2H), 2.21-1.70 (m, 4H), 1.52-0.73 (m, 3H). LCMS (M+H)⁺=563.4.

### Compounds Cell Killing in HCT116 Isogenic Pair

HCT116 cell line (ATCC, CCL-247) was isolated from the colon of an adult male with colon cancer. It has a mutation in codon 13 of the ras proto-oncogene and can be used as a positive control for PCR assays of mutation in this codon. HCT116-MTAP-KO was knocked-out-MTAP-gene based HCT116, and a single clone was passed for this assay. HCT116-mock-RNA-KO was knocked-out-mock-gene based HCT116 with MTAP wildtype genotype. The base medium for HCT116 isogenic pair is RPMI 1640, HEPES (Gibco, 22400105). To make the complete growth medium, add the following components to the base medium: fetal bovine serum to a final concentration of 10% (Gibco, 10099-141C). The cell line was grown in a humidified 5% CO₂ atmosphere at 37° C. and regularly tested for the presence of mycoplasma with MycoAlert™ PLUS Mycoplasma Detection Kit (Lonza, LT07-710).

### EXPERIMENTAL

Seed HCT116-mock-RNA-KO (4E2/well) or HCT116-MTAP-KO (4E2/well) cells into a 96-well plate (Greiner: 655090), 100 L/well.
Incubate at 37° C., 5% CO₂, overnight.
Add 50 μL fresh growth-medium containing serial dilution of compounds for a final compound concentration of 0-10 μM to each well.
Incubate at 37° C., 5% CO₂, 6 days.
The cell viability detected by Cell Titer-Glo (Promega, G7573). Add 70 μL of CellTiter-Glo® reagent in each well.
Incubate at room temperature for 10 minutes to stabilize luminescent signal.
Analyze with Microplate Reader (TECAN, SPARK) and fit IC₅₀ using Prism® 9.

The compounds disclosed herein showed cell killing activity values as in Table 1

TABLE 1
Cell killing IC₅₀ (nM) for the compounds disclosed herein

| | Cell killing | Cell killing | | Cell killing | Cell killing |
|---|---|---|---|---|---|
| | IC₅₀ | IC₅₀ (nM) in | | IC₅₀ | IC₅₀ (nM) in |
| | (nM) in | HCT116- | | (nM) in | HCT116- |
| | HCT116- | mock-RNA- | | HCT116- | mock-RNA- |
| Example | MTAP-KO | KO | Example | MTAP-KO | KO |
| 1 | 98 | >10000 | 2A | 98 | 3685 |
| 2B | >10000 | >10000 | 3 | 57 | 3866 |
| 4A | 85 | 2951 | 4B | 1540 | 5913 |
| 5 | 707 | >10000 | 6 | 2556 | >10000 |
| 7 | 285 | >10000 | 8 | 255 | 5254 |
| 9 | 222 | 4670 | 10 | 18 | 7231 |
| 11A | 57 | 2792 | 11B | 2088 | >10000 |
| 12 | 482 | 6006 | 13 | 62 | 3951 |
| 14 | 378 | >10000 | 15 | 942 | 5085 |
| 16 | 699 | >10000 | 17 | 253 | >10000 |
| 18 | 35 | 2224 | 19A | 17 | 2231 |
| 19B | 848 | >10000 | 20 | 215 | >1000 |
| 21 | 102 | 4009 | 22 | 130 | 7372 |
| 23 | 3992 | >10000 | 24 | 5257 | >10000 |
| 25 | 3690 | >10000 | 26 | 77 | 2784 |
| 27 | 415 | 7293 | 28 | 102 | 3211 |
| 29 | 1436 | >10000 | 30 | 254 | >10000 |
| 31 | 18 | 3147 | 32 | 140 | 7328 |
| 33 | 150 | 9796 | 34 | 66 | 3648 |
| 35 | 924 | >10000 | | | |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 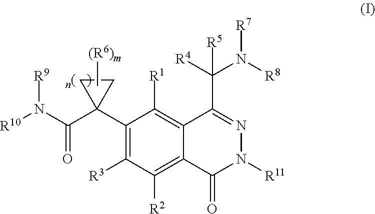
or a N-oxide thereof, or a pharmaceutically acceptable salt thereof, or a stereoisomer thereof, or a tautomer thereof, or a deuterated analog thereof, wherein:
n is 1, 2, 3 or 4;
m is 1, 2, 3, or 4;
R¹ and R² are each independently selected from hydrogen, halogen, —C₁₋₈alkyl, —C₃-C₈cycloalkyl, —CN, —OR^{1a}, —NR^{1a}R^{1b}, —COR^{1a}, —CO₂R^{1a}, —CONR^{1a}R^{1b} or —NR^{1a}COR^{1b}, wherein each of —C₁₋₈alkyl and —C₃-C₈cycloalkyl is optionally substituted with at least one substituent selected from halogen, —C₁₋₈alkoxy, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —N^{1c}COR^{1d}, —NR^{1c}CO₂R^{1d}, or —NR^{1c}SO₂R^{1d};
R^{1a} and R^{1b} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl; each of said —C₁₋₈alkyl, —C₂₋₈alkenyl, —C₂₋₈alkynyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R³, R⁴, R⁵ and R⁶ are each independently selected from hydrogen, halogen, —C₁₋₈alkyl, —C₃-C₈cycloalkyl, —CN, —OR^{3a}, —NR^{3a}R^{3b}, —COR^{3a}, —CO₂R^{3a}, —CONR^{3a}R^{3b} or —NR^{3a}COR^{3b}, wherein each of —C₁₋₈alkyl and —C₃-C₈cycloalkyl is optionally substituted with at least one substituent selected from halogen, —C₁₋₈alkoxy, —C₁₋₈alkyl, —C₃-C₈cycloalkyl, 3- to 8-membered heterocyclyl, C₆-C₁₂aryl, 5- to 12-membered heteroaryl, oxo, —CN, —OR^{3c}, —SO₂R^{3c}, —SO₂NR^{3c}R^{3d}, —COR^{3c}, —CO₂R^{3c}, —CONR^{3c}R^{3d}, —NR^{3c}R^{3d}, —NR^{3c}COR^{3d}, —NR^{3c}CO₂R^{3d}, or —NR^{3c}SO₂R^{3d};
R^{3a}, R^{3b}, R^{3c} and R^{3d} are each independently hydrogen, —C₁₋₈alkyl, C₃-C₈cycloalkyl, 3- to 8-membered heterocyclyl, —C₆-C₁₂aryl, or 5- to 12-membered heteroaryl; each of said —C₁₋₈alkyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁷ and R⁸ are each independently selected from hydrogen, —C₁₋₈alkyl, —C₃-C₈cycloalkyl or —C₆-C₁₂aryl, wherein each of —C₁₋₈alkyl, —C₃-C₈cycloalkyl and —C₆-C₁₂aryl is optionally substituted with at least one substituent selected from hydrogen, halogen, —C₁₋₈alkyl, —C₂₋₈alkenyl, —C₂₋₈alkynyl, —NR^{7a}R^{7b}, —OR^{7a}, oxo, —C₃-C₈cycloalkyl, 3- to 8-membered heterocyclyl, —C₆-C₁₂aryl, 5- to 12-membered heteroaryl, or —CN;
R^{7a} and R^{7b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, wherein each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{7c};
R^{7c} is independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl or —CN, wherein each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl is optionally substituted with at least one hydrogen, halogen, hydroxy, —C₁₋₈alkyl, —C₁₋₈alkoxy, —CN, —NH₂ or oxo;
R⁹ and R¹⁰ are each independently selected from hydrogen, —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{9a}; or
R⁹ and R¹⁰ together with the nitrogen atom to which they are attached, form a 3- to 8-membered unsaturated or saturated ring, said ring comprising 0-3 additional heteroatoms independently selected from nitrogen, oxygen or sulfur and said ring is optionally substituted with at least one substituent R^{9a};
R^{9a} is independently hydrogen, halogen, deuterium, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9b}, —SO₂R^{9b}, —SO₂NR^{9b}R^{9c}, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{9d}; or
two R^{9a} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent R^{9d};
R^{9b} and R^{9c} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{9e};
R^{9d} and R^{9e} are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9f}, —SO₂R^{9f}, —SO₂NR^{9f}R^{9g}, —COR^{9f}, —CO₂R^{9f}, —CONR^{9f}R^{9g}, —NR^{9f}R^{9g}, —NR^{9f}COR^{9g}, —NR^{9f}CO₂R^{9g}, —NR^{9f}SO₂R^{9g}, oxo, —SF₅ or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{9h};
R^{9f} and R^{9g} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R⁹ⁱ;
R^{9h} and R⁹ⁱ are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{9j}, —SO₂R^{9j}, —SO₂NR^{9j}R^{9k}, —COR^{9j}, —CO₂R^{9j}, —CONR^{9j}R^{9k}, —NR^{9j}R^{9k}, —NR^{9j}COR^{9k}, —NR^{9j}CO₂R^{9k}, —NR^{9j}SO₂R^{9k}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo;
R^{9j} and R^{9k} are each independently selected from hydrogen, —C₁₋₈alkyl, —C₃-C₈cycloalkyl, 3- to 12-membered heterocyclyl, —C₆-C₁₂aryl and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo.

2. The compound of claim 1, wherein the compound is formula (IIa) or (IIb): 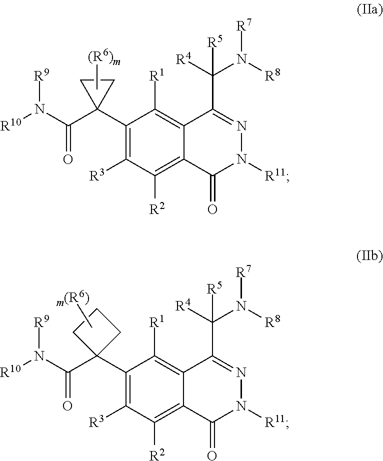
preferably, the compound is selected from formula (IIc) or (IId):

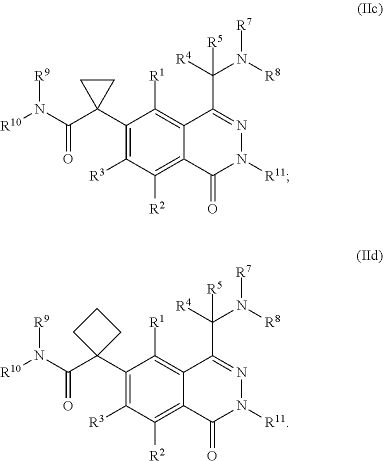

3. The compound of claim 1, wherein the compound is formula (IIIa): 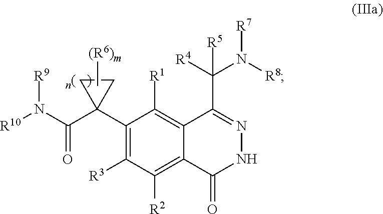
preferably, the compound is selected from formula (IIIb): 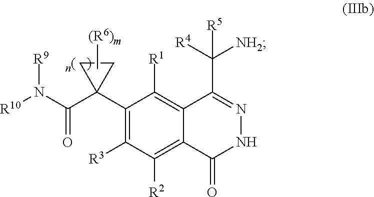
more preferably, the compound is selected from formula (IIIc): 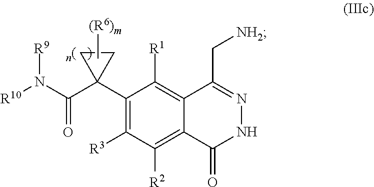
more preferably, the compound is selected from formula (IIId):

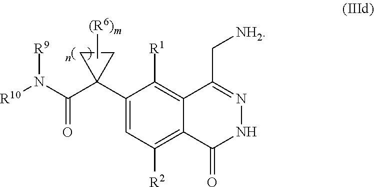

4. The compound of claim 1, wherein the compound is formula (IVa) or (IVb): 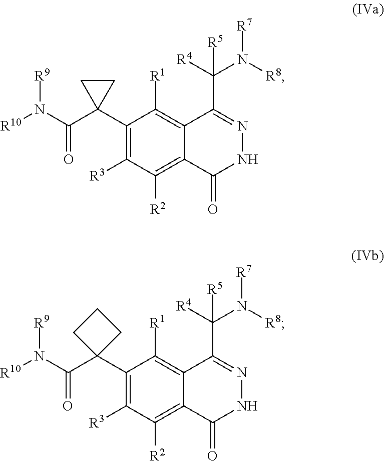
preferably, the compound is selected from formula (IVc) or (IVd): 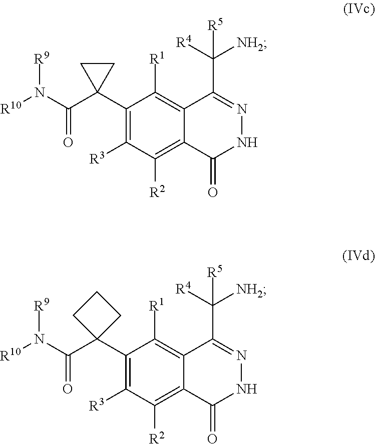
more preferably, the compound is selected from formula (IVe) or (IVf): 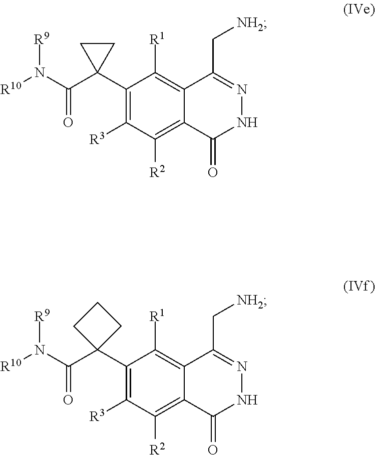
even more preferably, the compound is selected from formula (IVg) or (IVh): 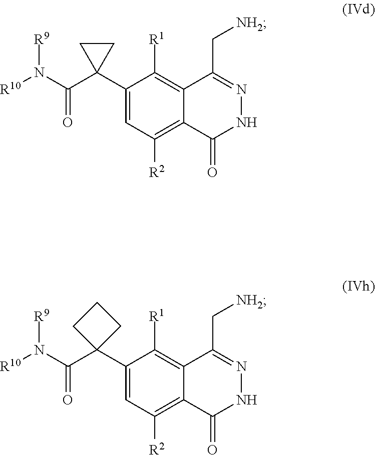
even more preferably, the compound is selected from formula (IVi) or (IVj):

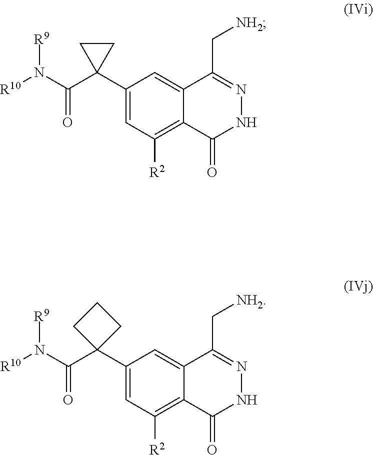

5. The compound of claim 1, wherein the compound is formula (Va): 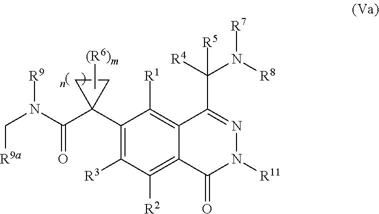
preferably, the compound is selected from formula (Vb) or (Vc): 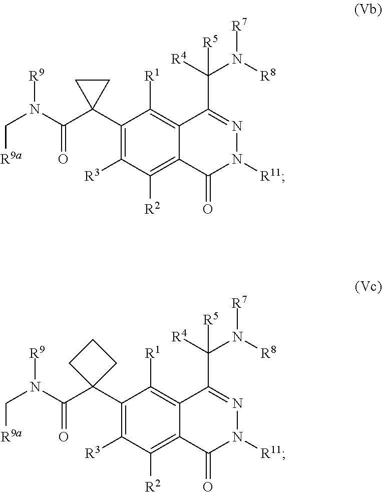
more preferably, the compound is selected from formula (Vd) or (Ve): 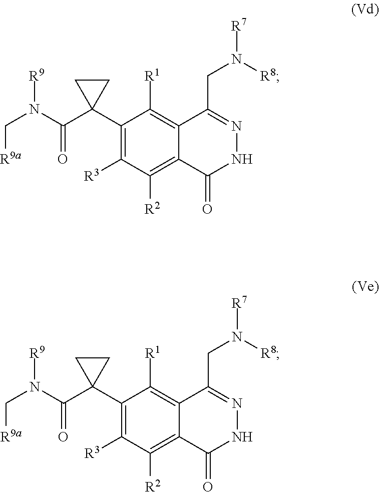
even more preferably, the compound is selected from formula (Vf) or (Vg): 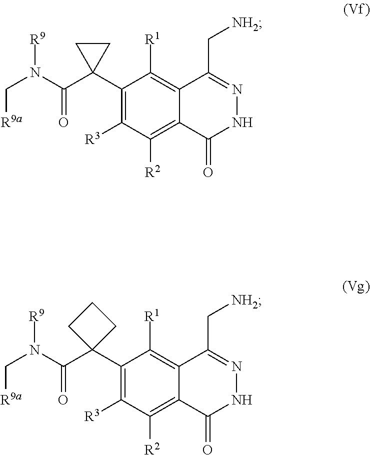
even more preferably, the compound is selected from formula (Vh) or (Vi): 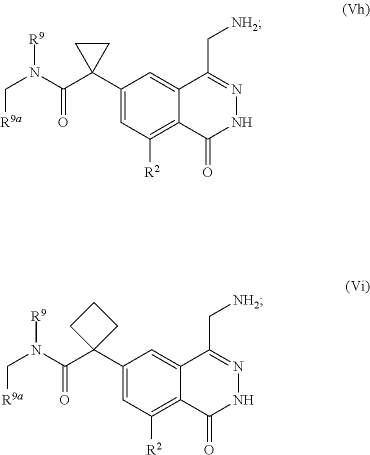
wherein, R^{9a} is as defined as claim 1.

6. The compound of anyone of preceding claims, wherein R¹ and R² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{1a}, —NR^{1a}R^{1b}, —COR^{1a}, —CO₂R^{1a}, —CONR^{1a}R^{1b} or —NR^{1a}COR^{1b}, wherein each of methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent selected from —F, —Cl, —Br, —I, methoxy, ethoxy, propoxy, butoxy, pentoxy, hexoxy, heptoxy, octoxy, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
R^{1c} and R^{1d} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

7. The compound of anyone of preceding claims, wherein R¹ and R² are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{1a}, —NR^{1a}R^{1b}, —COR^{1a}, —CO₂R^{1a}, —CONR^{1a}R^{1b} or —NR^{1a}COR^{1b};
R^{1a} and R^{1b} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R¹ and R² are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN;
more preferably, R¹ and R² are each independently selected from hydrogen, —F, —Cl, methyl, ethyl, propyl (iso-propyl or n-propyl), butyl (n-butyl, sec-butyl, iso-butyl or tert-butyl), cyclopropyl, cyclobutyl,
even more preferably, R¹ and R² are each independently selected from hydrogen or methyl.

8. The compound of anyone of preceding claims, wherein R³, R⁴, R⁵ and R⁶ are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OR^{3a}, —NR^{3a}R^{3b}, —COR^{3a}, —CO₂R^{3a}, —CONR^{3a}R^{3b} or —NR^{3a}COR^{3b}, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl or cyclooctyl is optionally substituted with at least one substituent selected from —F, —Cl, —Br, —I, methoxy, ethoxy, propoxy, butoxy, pentoxy, hexoxy, heptoxy, octoxy, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{3c}, —SO₂R^{3c}, —SO₂NR^{3c}R^{3d}, —COR^{3c}, —CO₂R^{3c}, —CONR^{3c}R^{3d}, —NR^{3c}R^{3d}, —NR^{3c}COR^{3d}, —NR^{3c}CO₂R^{3d}, or —NR^{3c}SO₂R^{3d};
R^{3a}, R^{3b}, R^{3c} and R^{3d} are each independently hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R³, R⁴, R and R⁶ are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OR^{3a}, —NR^{3a}R^{3b}, —COR^{3a}, —CO₂R^{3a}, —CONR^{3a}R^{3b} or —NR^{3a}COR^{3b};
R^{3a} and R^{3b} are each independently hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
more preferably, R³, R⁴, R⁵ and R⁶ are each independently selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —CN, —OH, methoxy, ethoxy, propoxy, butoxy, pentoxy, hexoxy, heptoxy, octoxy;
even more preferably, R³, R⁴, R⁵ and R⁶ are each independently selected from hydrogen, —F, —Cl, methyl, ethyl, propyl (iso-propyl or n-propyl), butyl (n-butyl, sec-butyl, iso-butyl or tert-butyl);
even more preferably, R³, R⁴, R⁵ and R⁶ are each independently selected from hydrogen.

9. The compound of anyone of preceding claims, wherein R⁷ and R⁸ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl is optionally substituted with at least one substituent selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, —NR^{7a}R^{7b}, —OR^{7a}, oxo, cyclopropyl, cyclobutyl, cyclopentyl, cyclohexyl, cycloheptyl, cyclooctyl 3- to 8-membered heterocyclyl, phenyl, 5- to 12-membered heteroaryl, or —CN;
R^{7a} and R^{7b} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7c};
R^{7c} is independently —F, —Cl, —Br, —I, hydroxy, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN, wherein each of said hydroxy, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one hydrogen, halogen, hydroxy, —C₁₋₈alkyl, —C₁₋₈alkoxy, —CN, —NH₂ or oxo;
preferably, R⁷ and R⁸ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl, wherein each of methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl or phenyl is optionally substituted with at least one substituent selected from hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, —NR^{7a}R^{7b}, —OR^{7a}, oxo, cyclopropyl, cyclobutyl, cyclopentyl, cyclohexyl, cycloheptyl, cyclooctyl 3- to 8-membered heterocyclyl, phenyl, 5- to 12-membered heteroaryl, or —CN;
R^{7a} and R^{7b} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R⁷ and R⁸ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl;
even more preferably, R⁷ and R⁸ are each independently selected from hydrogen.

10. The compound of anyone of the preceding claims, wherein R⁹ and R¹⁰ are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl and 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur and said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, naphthalenyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9b}, —SO₂R^{9b}, —SO₂NR^{9b}R^{9c}, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl or 5- to 12-membered heteroaryl is optionally substituted with at least one substituent R^{9d}; or
two R^{9a} together with the atom(s) to which they are attached, form a 3-, 4-, 5-, 6-, 7- or 8-membered unsaturated or saturated ring, said ring comprising 0, 1, 2 or 3 heteroatoms independently selected from nitrogen, oxygen or sulfur and said ring is optionally substituted with at least one substituent R^{9d};
R^{9b} and R^{9c} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl or 5- to 12-membered heteroaryl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9f}, —SO₂R^{9f}, —SO₂NR^{9f}R^{9g}, —COR^{9f}, —CO₂R^{9f}, —CONR^{9f}R^{9g}, —NR^{9f}R^{9g}, —NR^{9f}COR^{9g}, —NR^{9f}CO₂R^{9g}, —NR^{9f}SO₂R^{9g}, oxo, —SF₅ or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, and 5- to 12-membered heteroaryl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl, naphthalenyl or 5- to 12-membered heteroaryl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 12-membered heterocyclyl, phenyl, naphthalenyl or 5- to 12-membered heteroaryl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl, 5- to 12-membered heteroaryl, —OR^{9j}, —SO₂R^{9j}, —SO₂NR^{9j}R^{9k}, —COR^{9j}, —CO₂R^{9j}, —CONR^{9j}R^{9k}, —NR^{9j}R^{9k}, —NR^{9j}COR^{9k}, —NR^{9j}CO₂R^{9k}, —NR^{9j}SO₂R^{9k}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, —C₃-C₁₂cycloalkenyl, 3- to 12-membered heterocyclyl, phenyl and 5- to 12-membered heteroaryl is optionally substituted with at least one substituent —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OH, —NH₂ or oxo;
R^{9j} and R^{9k} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl,
wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 12-membered heterocyclyl, phenyl and 5- to 12-membered heteroaryl is optionally substituted with at least one substituent —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, —OH, —NH₂ or oxo.

11. The compound of anyone of the preceding claims, wherein R⁹ and R¹⁰ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, tetrahydrofuropyridinyl —OR^{9b}, —SO₂R^{9b}, —SO₂NR^{9b}R^{9c}, —COR^{9b}, —CO₂R^{9b}, —CONR^{9b}R^{9c}, —NR^{9b}R^{9c}, —NR^{9b}COR^{9c}, —NR^{9b}CO₂R^{9c}, —NR^{9b}SO₂R^{9c}, oxo, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} and R^{9c} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —COR^{9f}, —CO₂R^{9f}, —CONR^{9f}R^{9g}, —NR^{9f}R^{9g}, —NR^{9f}COR^{9g}, —NR^{9f}CO₂R^{9g}, oxo, —SF₅, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9j}, —COR^{9j}, —CO₂R^{9j}, —CONR^{9j}R^{9k}, —NR^{9j}R^{9k}, —NR^{9j}COR^{9k}, —NR^{9j}CO₂R^{9k}, oxo, or —CN, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —CN, —OH, —NH₂ or oxo;
R^{9j} and R^{9k} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —CN, —OH, —NH₂ or oxo;
preferably, R⁹ and R¹⁰ are each independently selected from hydrogen, methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl (preferably, 2,3-dihydrofuro[2,3-b]pyridinyl), tetrahydrofuropyridinyl, —OR^{9b} or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, naphthalenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} is each independently hydrogen, methyl, ethyl, propyl (iso-propyl or n-propyl), cyclopropyl or cyclobutyl;
R^{9d} is each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —NR^{9f}R⁹, —NR^{9f}COR^{9g}, oxo, —SO₂R^{9f}, —SF₅, or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} and R^{9g} are each independently selected from hydrogen, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, triazolyl (preferably, 1H-1,2,4-triazolyl, 4H-1,2,4-triazolyl), pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OH, or —CN;
more preferably, R⁹ and R¹⁰ are each independently selected from hydrogen, methyl, ethyl, propyl (iso-propyl or n-propyl), cyclopropyl, cyclobutyl, cyclopentyl, cyclohexyl, phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), phenyl, chromanyl, isochromanyl, dihydropyranopyridinyl (preferably, 5,8-dihydro-6H-pyrano[3,4-b]pyridinyl, 3,4-dihydro-2H-pyrano[3,2-b]pyridine or 7,8-dihydro-5H-pyrano[4,3-b]pyridinyl), 6,7-dihydro-5H-cyclopenta[b]pyridinyl, pyrimidinyl, tetrahydro-2H-pyranyl, pyridazinyl, pyrazinyl, dihydrofuropyridinyl (preferably, 2,3-dihydrofuro[2,3-b]pyridinyl), tetrahydronaphthalenyl (preferably, 1,2,3,4-tetrahydronaphthalenyl), benzo[d]thiazolyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 6-membered unsaturated or saturated ring, said ring is optionally substituted with at least one substituent R^{9a};
R^{9a} is independently hydrogen, —F, —Cl, —Br, —I, methyl, ethyl, propyl (iso-propyl or n-propyl), cyclopropyl, cyclobutyl, cyclopentyl, cyclohexyl, phenyl, naphthalenyl, pyrimidinyl, pyridazinyl, pyrazinyl, dihydrofuropyridinyl (preferably, 2,3-dihydrofuro[2,3-b]pyridinyl), benzo[d]thiazolyl, pyridinyl, quinolinyl, isoquinolinyl, thiazolyl, —OR^{9b} or —CN wherein each of said methyl, ethyl, propyl (iso-propyl or n-propyl), phenyl, naphthalenyl, pyrimidinyl, pyridazinyl, pyrazinyl, dihydrofuropyridinyl (preferably, 2,3-dihydrofuro[2,3-b]pyridinyl), benzo[d]thiazolyl, pyridinyl, quinolinyl, isoquinolinyl or thiazolyl is optionally substituted with at least one substituent R^{9d};
R^{9b} is each independently hydrogen, methyl, ethyl, propyl (iso-propyl or n-propyl), cyclopropyl or cyclobutyl;
R^{9d} is each independently hydrogen, —F, —Cl, —Br, —I, methyl, ethyl, propyl (iso-propyl or n-propyl), butyl (n-butyl, sec-butyl, iso-butyl or tert-butyl), phenyl, pyrimidinyl, tetrahydro-2H-pyranyl, pyridazinyl, pyrazinyl, triazolyl (preferably, 1H-1,2,4-triazolyl, 4H-1,2,4-triazolyl), pyridinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —SF₅ or —CN, wherein each of said methyl, ethyl, propyl (iso-propyl or n-propyl), butyl (n-butyl, sec-butyl, iso-butyl or tert-butyl), phenyl, pyrimidinyl, tetrahydro-2H-pyranyl, pyridazinyl, pyrazinyl, triazolyl (preferably, 1H-1,2,4-triazolyl, 4H-1,2,4-triazolyl), pyridinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} is each independently selected from hydrogen, methyl, ethyl, propyl (iso-propyl or n-propyl), cyclopropyl, cyclobutyl, cyclopentyl, cyclohexyl or phenyl, wherein each of said methyl, ethyl, propyl (iso-propyl or n-propyl), cyclopropyl, cyclobutyl, cyclopentyl, cyclohexyl or phenyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, —OH, or —CN.

12. The compound of anyone of preceding claims, wherein R⁹ and R¹⁰ are each independently selected from —C₁₋₈alkyl, —C₃-C₈cycloalkyl, —C₃-C₁₂cycloalkenyl and 3- to 12-membered heterocyclyl, wherein each of said —C₁₋₈alkyl, —C₃-C₈cycloalkyl, —C₃-C₁₂cycloalkenyl and 3- to 12-membered heterocyclyl is optionally substituted with at least one substituent R^{9a}; or
R⁹ and R¹⁰ together with the nitrogen atom to which they are attached, form a 3- to 8-membered unsaturated or saturated ring, said ring comprising 0-3 additional heteroatoms independently selected from nitrogen, oxygen or sulfur and said ring is optionally substituted with at least one substituent R^{9a};
R^{9a} is independently hydrogen, halogen, deuterium, —C₁₋₈alkyl, —C₃-C₈cycloalkyl, —C₆-C₁₂aryl, 5- to 12-membered heteroaryl, —OR^{9b} or —CN, wherein each of said —C₁₋₈alkyl, —C₃-C₈cycloalkyl, —C₆-C₁₂aryl and 5- to 12-membered heteroaryl is optionally substituted with at least one substituent R^{9d}; or
two R^{9a} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent R^{9d};
R^{9b} is each independently selected from hydrogen, —C₁₋₈alkyl, wherein said —C₁₋₈alkyl is optionally substituted with at least one substituent R^{9e};
R^{9d} and R^{9e} are each independently hydrogen, halogen, —C₁₋₈alkyl, —C₃-C₈cycloalkyl, —C₆-C₁₂aryl, 5- to 12-membered heteroaryl, —OR^{9f}, —SO₂R^{9f}, —SF₅ or —CN, wherein each of said —C₁₋₈alkyl, —C₆-C₁₂aryl and 5- to 12-membered heteroaryl is optionally substituted with at least one substituent R^{9h};
R^{9f} is each independently selected from hydrogen, —C₁₋₈alkyl and —C₃-C₈cycloalkyl, —C₆-C₁₂aryl, wherein each of said —C₁₋₈alkyl, —C₆-C₁₂aryl, and —C₃-C₈cycloalkyl is optionally substituted with at least one substituent R⁹ⁱ;
R^{9h} and R⁹ⁱ are each independently hydrogen, halogen, —C₁₋₈alkyl or —CN;
preferably, R⁹ and R¹⁰ are each independently selected from methyl, ethyl, propyl (iso-propyl or n-propyl), butyl (n-butyl, sec-butyl, iso-butyl or tert-butyl), cyclopropyl, cyclobutyl, cyclopentyl, cyclohexyl, chromanyl, isochromanyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl, wherein each of said methyl, ethyl, propyl (iso-propyl or n-propyl), butyl (n-butyl, sec-butyl, iso-butyl or tert-butyl), cyclopropyl, cyclobutyl, cyclopentyl, cyclohexyl, chromanyl, isochromanyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9a}; or
R⁹ and R¹⁰ together with the nitrogen atom to which they are attached, form a 3-, 4-, 5-, 6-, 7- or 8-membered unsaturated or saturated ring, said ring comprising 0, 1, 2 or 3 additional heteroatoms independently selected from nitrogen, oxygen or sulfur; said ring is optionally substituted with at least one substituent R^{9a};
R^{9a} is independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, pyrimidinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl (preferably, 2,3-dihydrofuro[2,3-b]pyridinyl), tetrahydrofuropyridinyl —OR^{9b} or —CN, wherein each of said methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, pyrimidinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9d}; or
two R^{9a} together with the atom(s) to which they are attached, form a 3-, 4-, 5- or 6-membered unsaturated or saturated ring, said ring comprising 0, 1, 2 or 3 heteroatoms independently selected from nitrogen, oxygen or sulfur; said ring is optionally substituted with at least one substituent R^{9d};
R^{9b} is each independently selected from hydrogen, methyl, ethyl, propyl (iso-propyl or n-propyl), butyl (n-butyl, sec-butyl, iso-butyl or tert-butyl), pentyl, hexyl, heptyl and octyl, wherein each of said methyl, ethyl, propyl (iso-propyl or n-propyl), butyl (n-butyl, sec-butyl, iso-butyl or tert-butyl), pentyl, hexyl, heptyl and octyl is optionally substituted with at least one substituent R^{9e};
R^{9d} and R^{9e} are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, cyclopropyl, cyclobutyl, cyclopentyl, cyclohexyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl, tetrahydrofuropyridinyl, —OR^{9f}, —SO₂R^{9f}, —SF₅ or —CN, wherein each of said methyl, ethyl, propyl (iso-propyl or n-propyl), butyl (n-butyl, sec-butyl, iso-butyl or tert-butyl), pentyl, hexyl, phenyl, pyridinyl, quinolinyl, isoquinolinyl, pyrazolyl, imidazolyl, thiazolyl, tetrahydropyranyl, tetrahydroquinolinyl, tetrahydronaphthyl, dihydrofuropyridinyl or tetrahydrofuropyridinyl is optionally substituted with at least one substituent R^{9h};
R^{9f} is each independently selected from hydrogen, methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl or naphthalenyl, wherein each of said methyl, ethyl, propyl (iso-propyl or n-propyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, phenyl or naphthalenyl is optionally substituted with at least one substituent R⁹ⁱ;
R^{9h} and R⁹ⁱ are each independently hydrogen, —F, —Cl, —Br, —I, methyl, ethyl, propyl (iso-propyl or n-propyl), butyl (n-butyl, sec-butyl, iso-butyl or tert-butyl), pentyl, hexyl, heptyl, octyl or —CN.

13. The compound of anyone of preceding claims, wherein R⁹ and R¹⁰ are each independently selected from -Me, -Et, —Pr (-nPr or -isoPr), -Bu 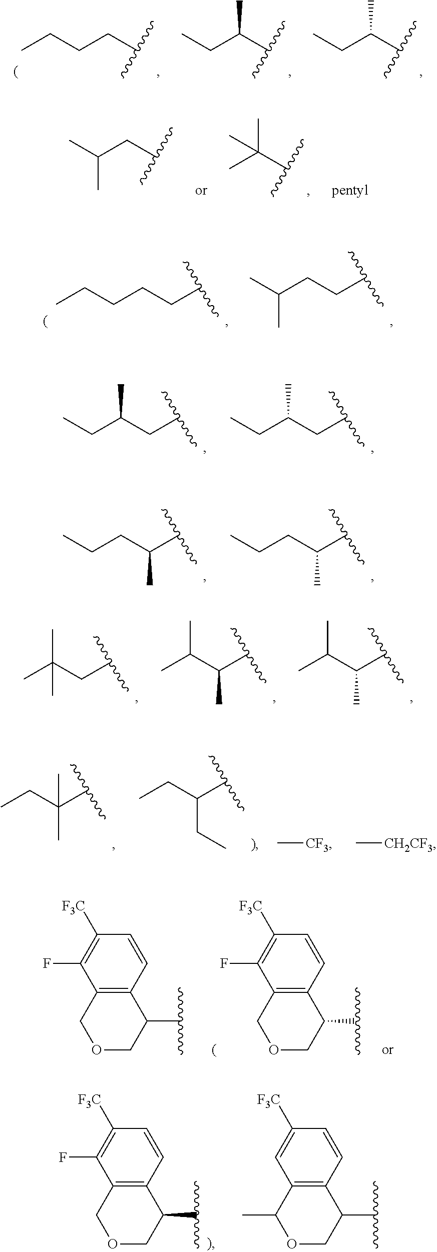 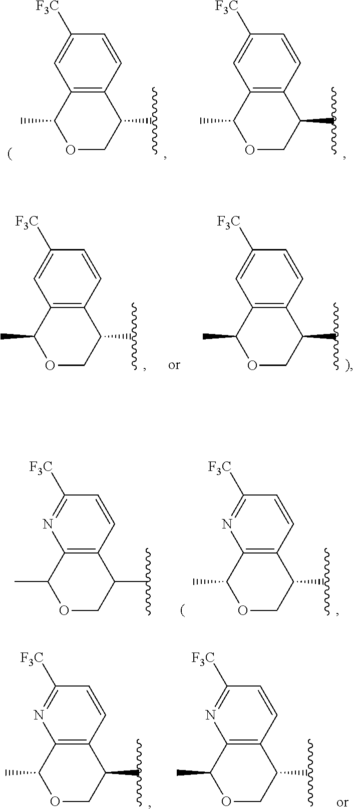 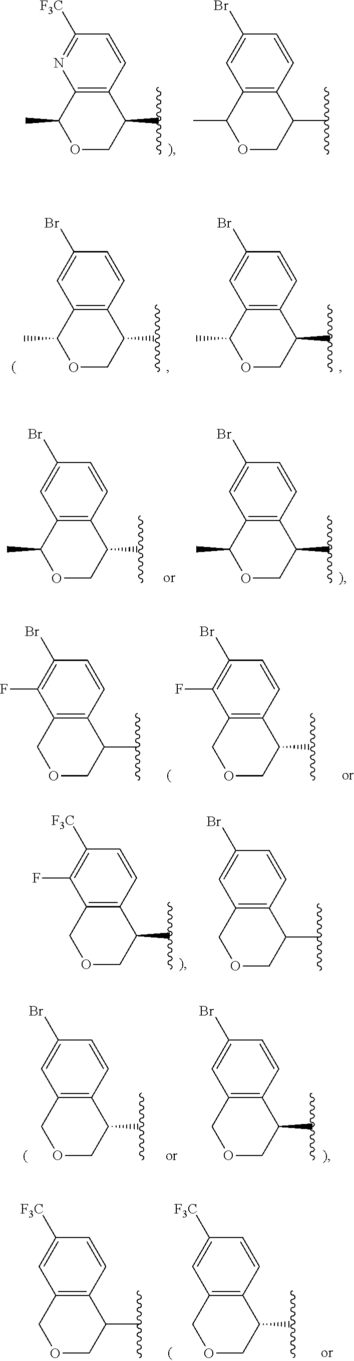 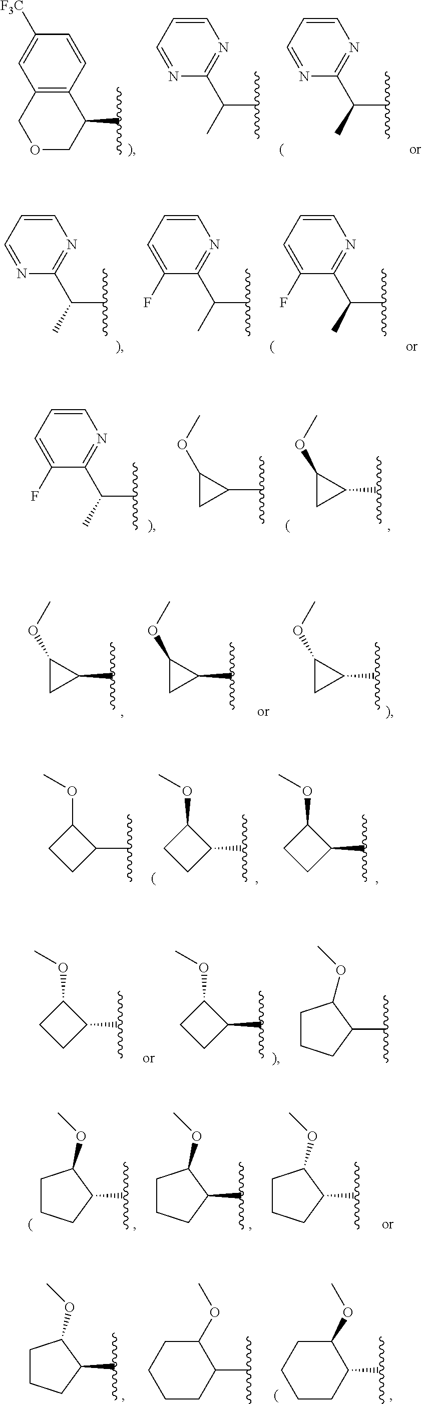 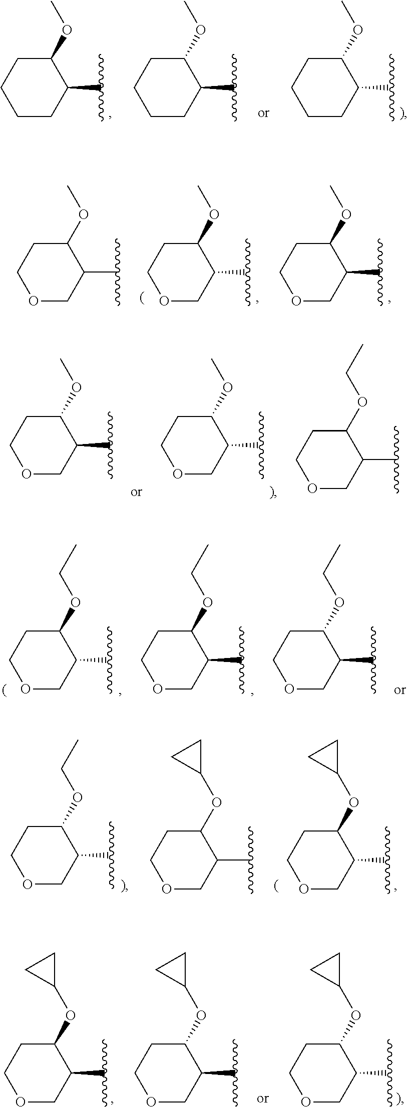 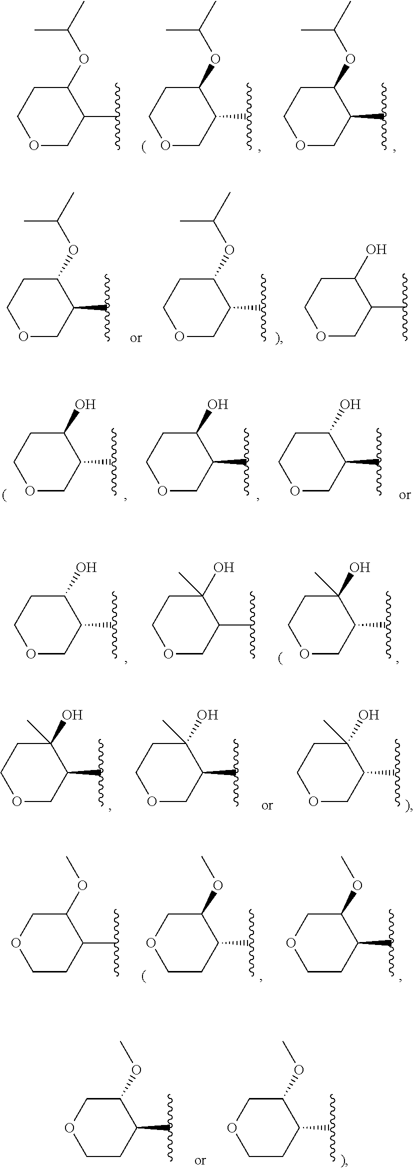 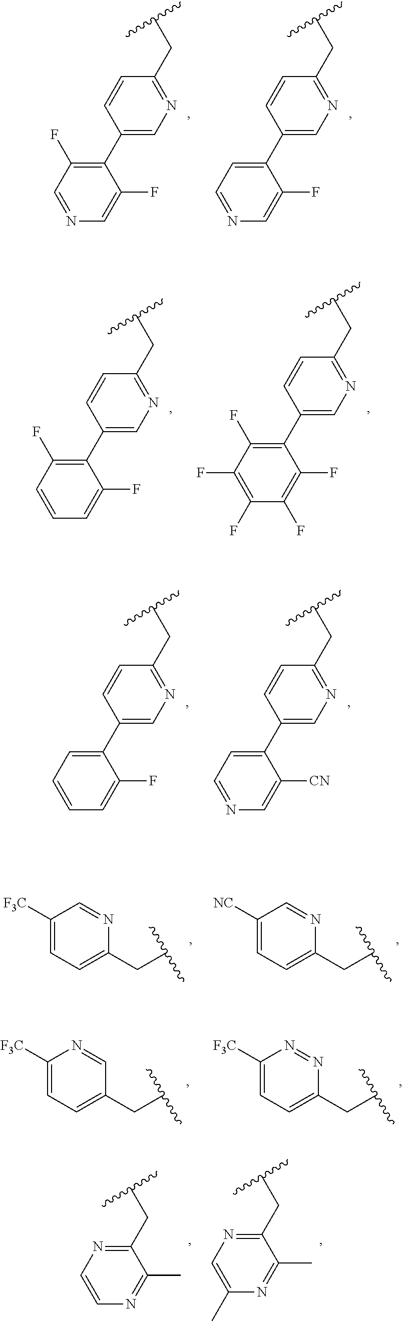 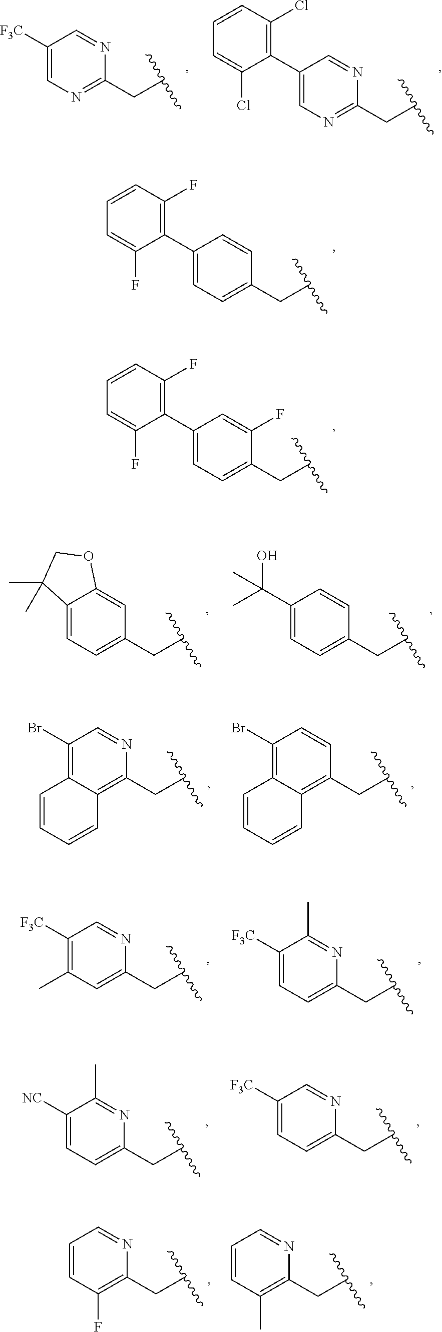 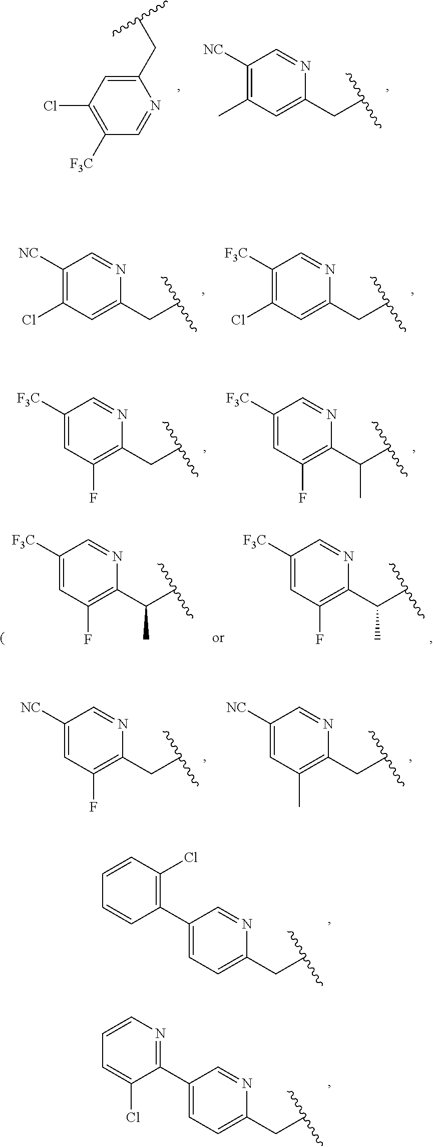 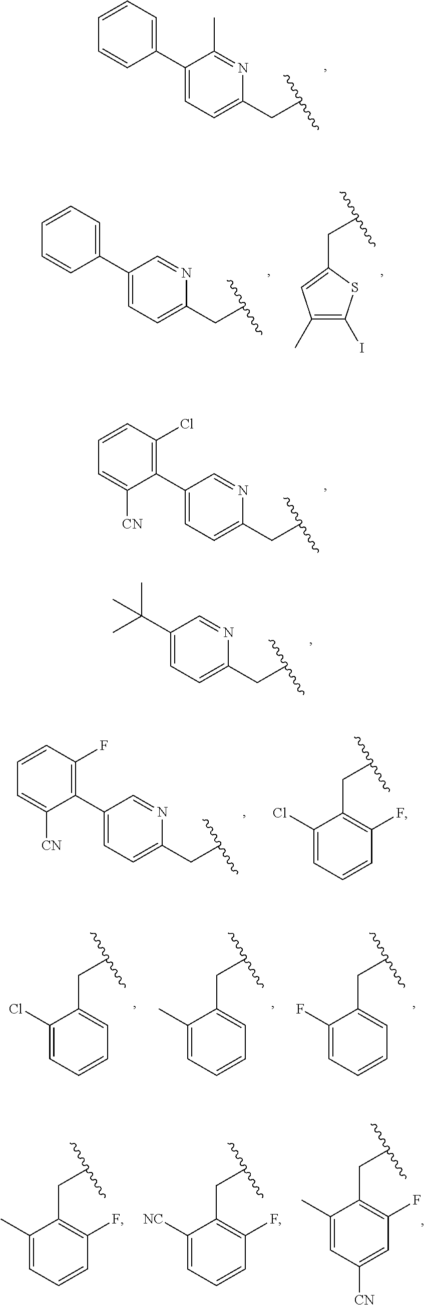 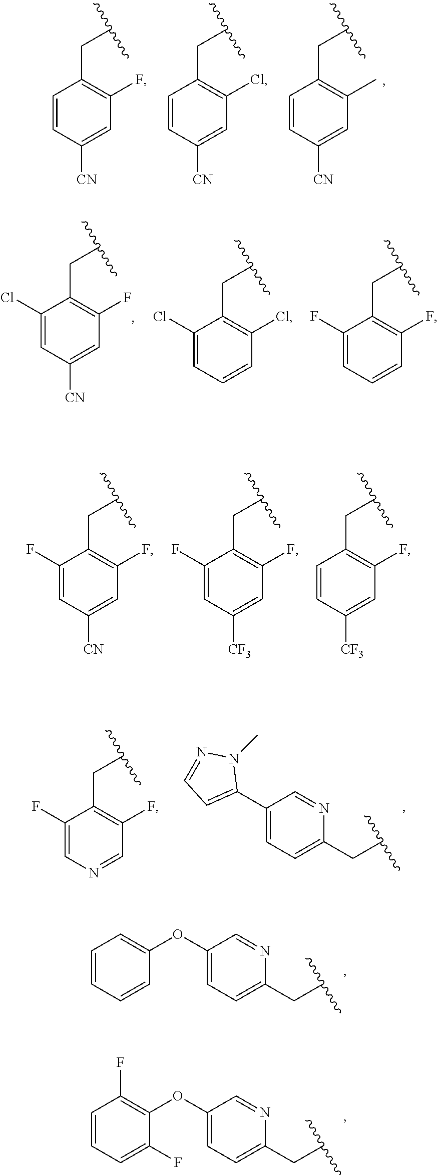 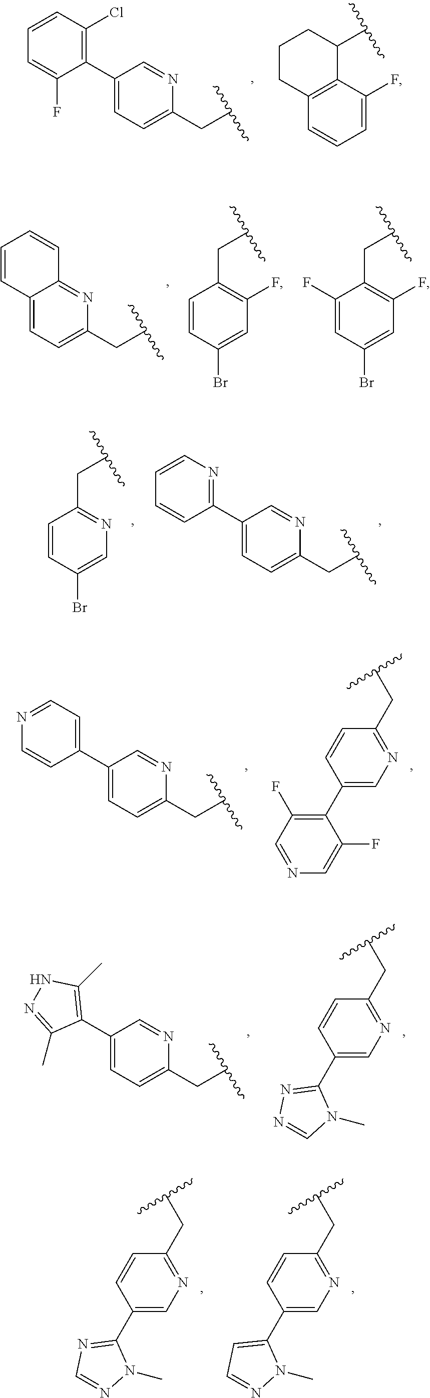 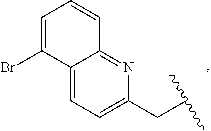 [SMILES: *Cc1ccc2c(Br)cccc2n1] 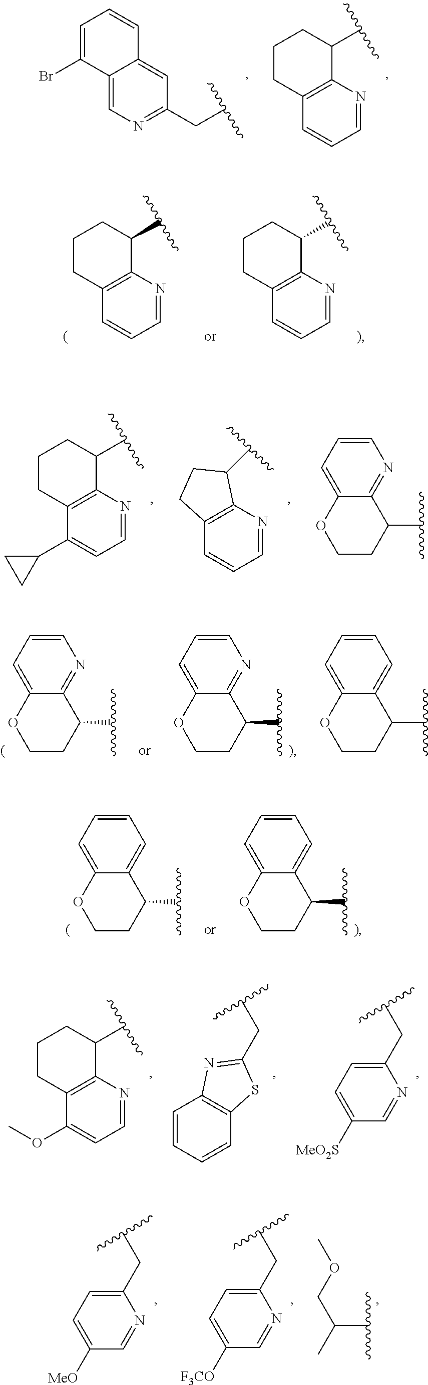 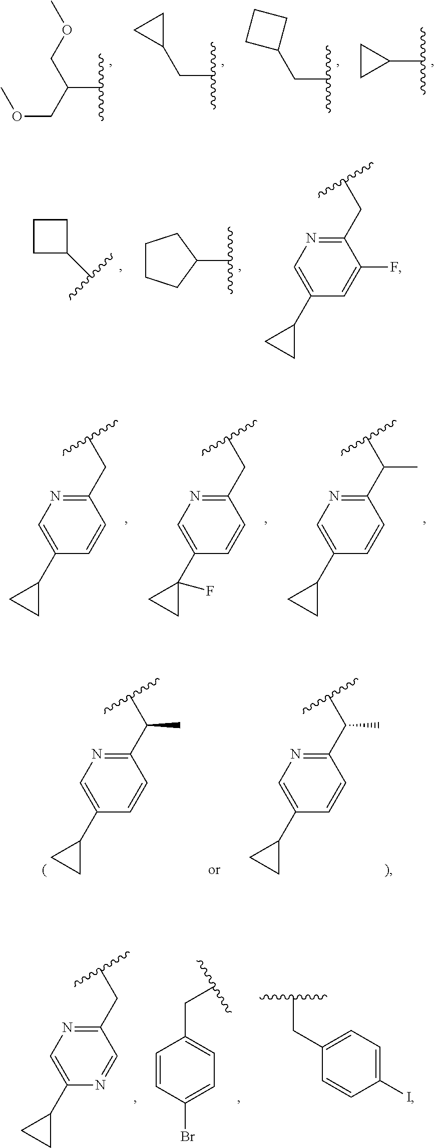 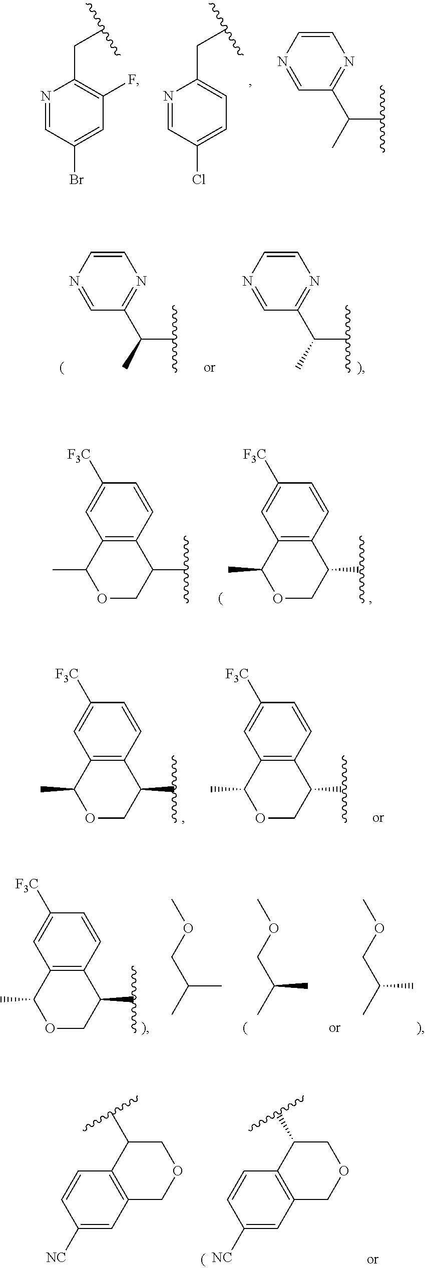 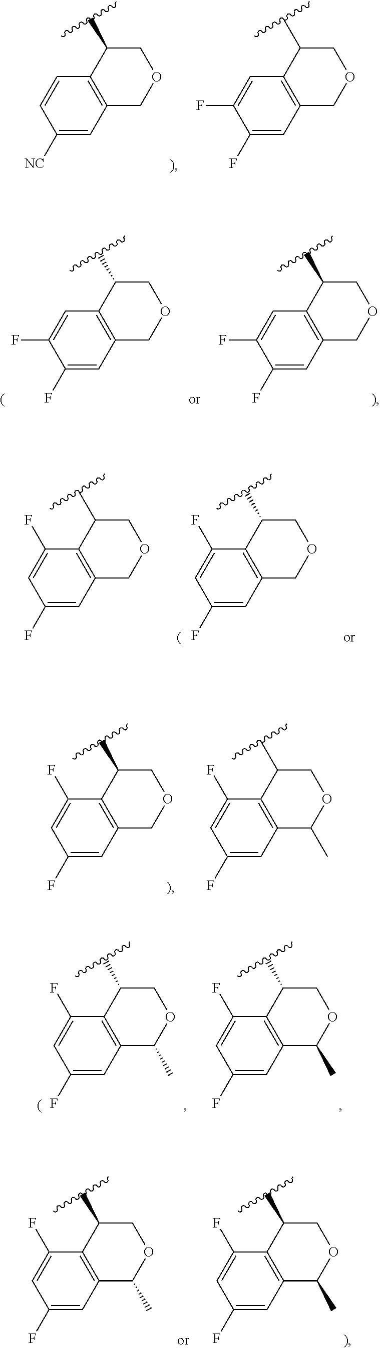 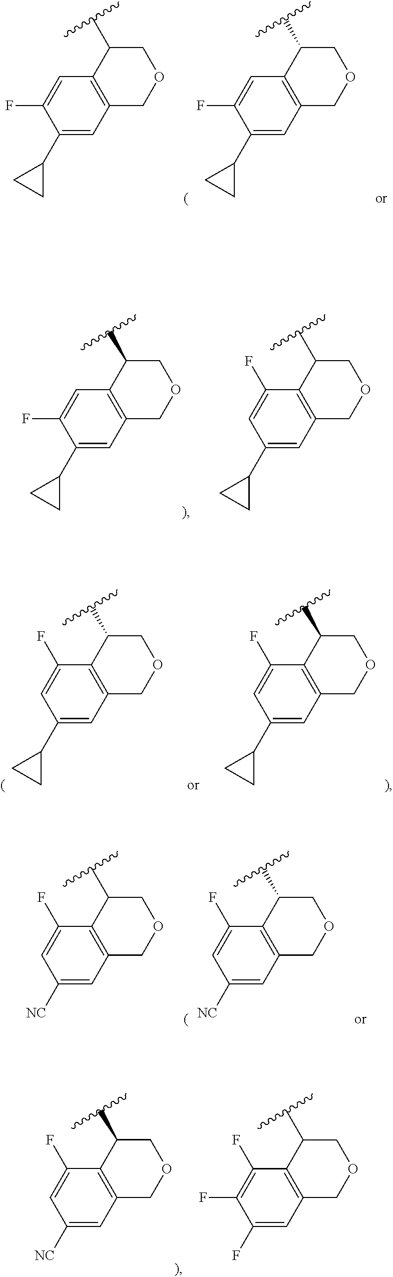 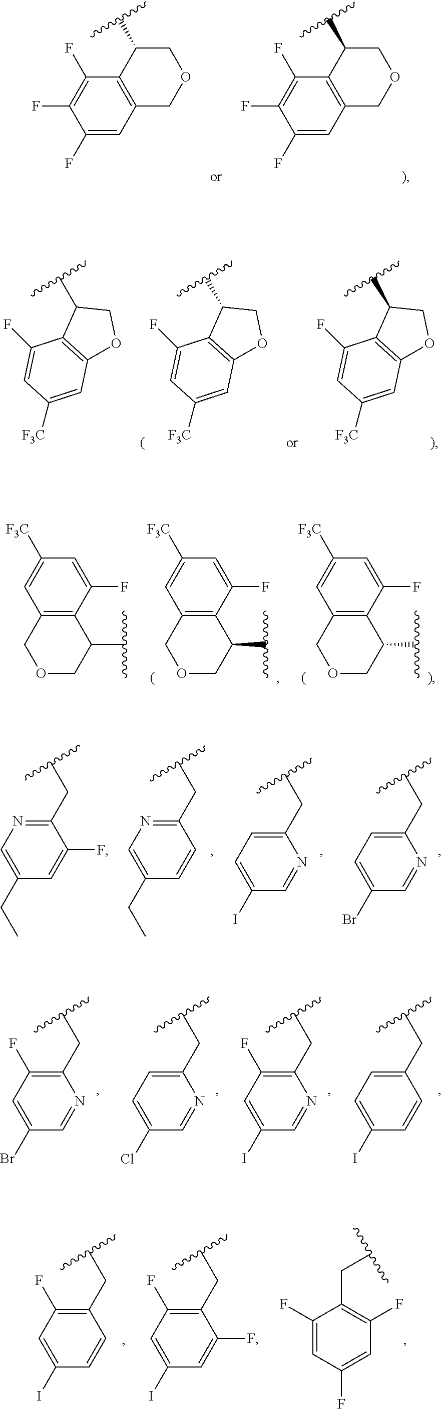 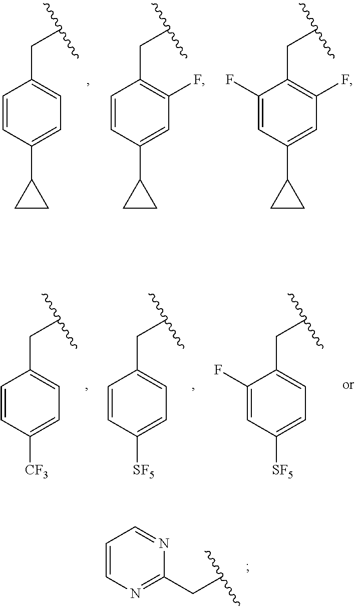
R⁹ and R¹⁰ together with the nitrogen atom to which they are attached, form

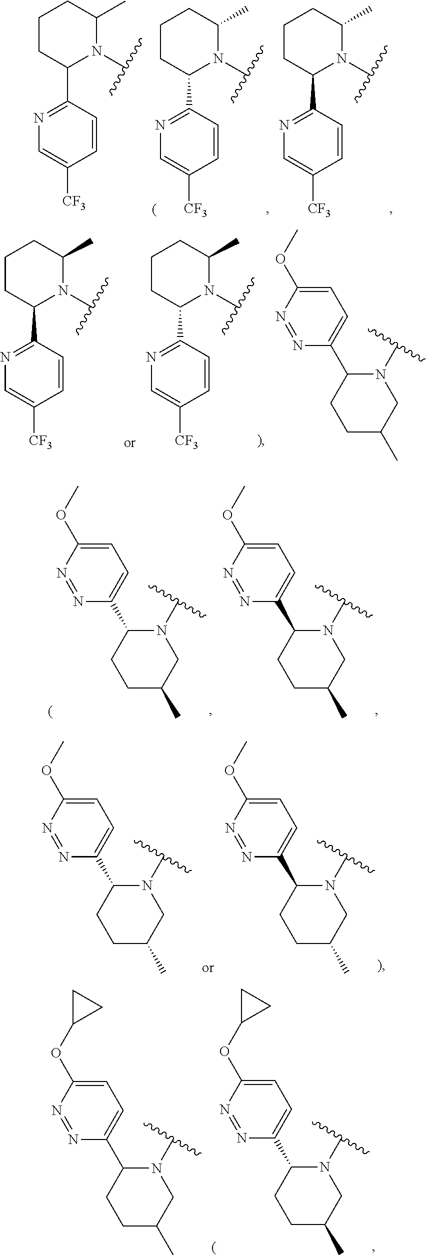

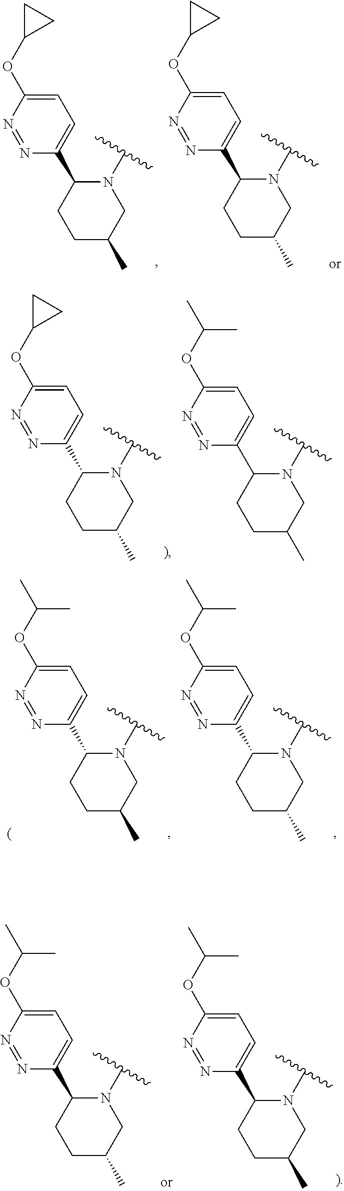

14. The compound of any one of the preceding claims, wherein the compound is selected from

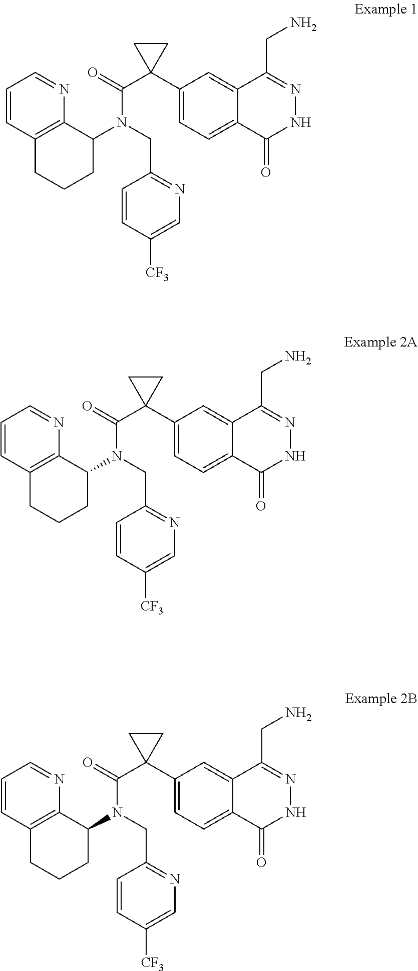 [SMILES: NCc1n[nH]c(=O)c2ccc(C3(C(=O)N(Cc4ccc(C(F)(F)F)cn4)C4CCCc5cccnc54)CC3)cc12 ; NCc1n[nH]c(=O)c2ccc(C3(C(=O)N(Cc4ccc(C(F)(F)F)cn4)[C@@H]4CCCc5cccnc54)CC3)cc12 ; NCc1n[nH]c(=O)c2ccc(C3(C(=O)N(Cc4ccc(C(F)(F)F)cn4)[C@H]4CCCc5cccnc54)CC3)cc12]

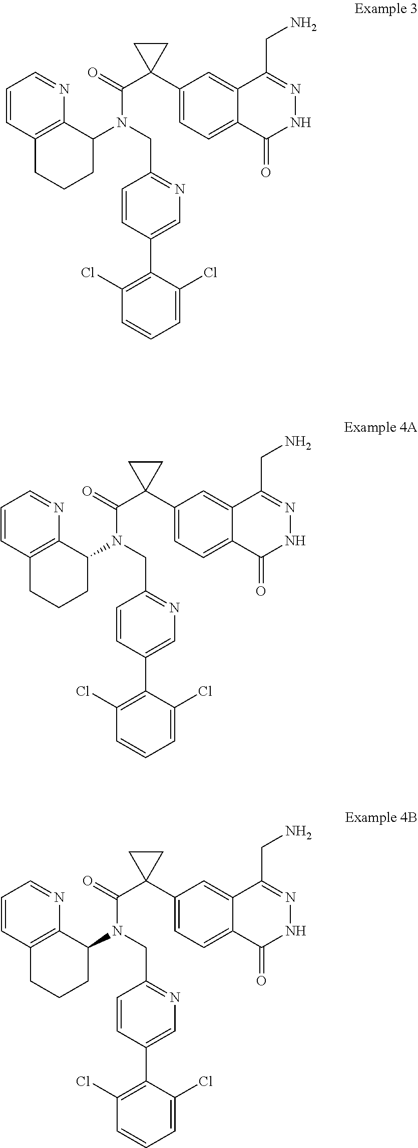 [SMILES: NCc1n[nH]c(=O)c2ccc(C3(C(=O)N(Cc4ccc(-c5c(Cl)cccc5Cl)cn4)C4CCCc5cccnc54)CC3)cc12 ; NCc1n[nH]c(=O)c2ccc(C3(C(=O)N(Cc4ccc(-c5c(Cl)cccc5Cl)cn4)[C@@H]4CCCc5cccnc54)CC3)cc12 ; NCc1n[nH]c(=O)c2ccc(C3(C(=O)N(Cc4ccc(-c5c(Cl)cccc5Cl)cn4)[C@H]4CCCc5cccnc54)CC3)cc12]

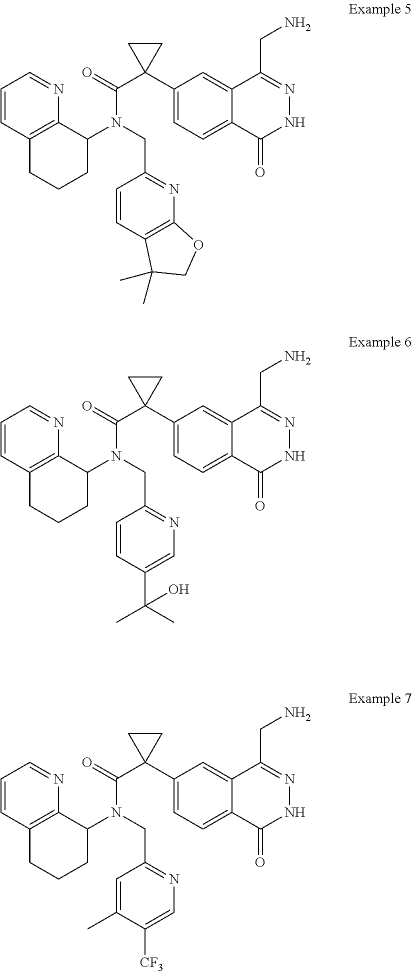 [SMILES: CC1(C)COc2nc(CN(C(=O)C3(c4ccc5c(=O)[nH]nc(CN)c5c4)CC3)C3CCCc4cccnc43)ccc21 ; CC(C)(O)c1ccc(CN(C(=O)C2(c3ccc4c(=O)[nH]nc(CN)c4c3)CC2)C2CCCc3cccnc32)nc1 ; Cc1cc(CN(C(=O)C2(c3ccc4c(=O)[nH]nc(CN)c4c3)CC2)C2CCCc3cccnc32)ncc1C(F)(F)F]

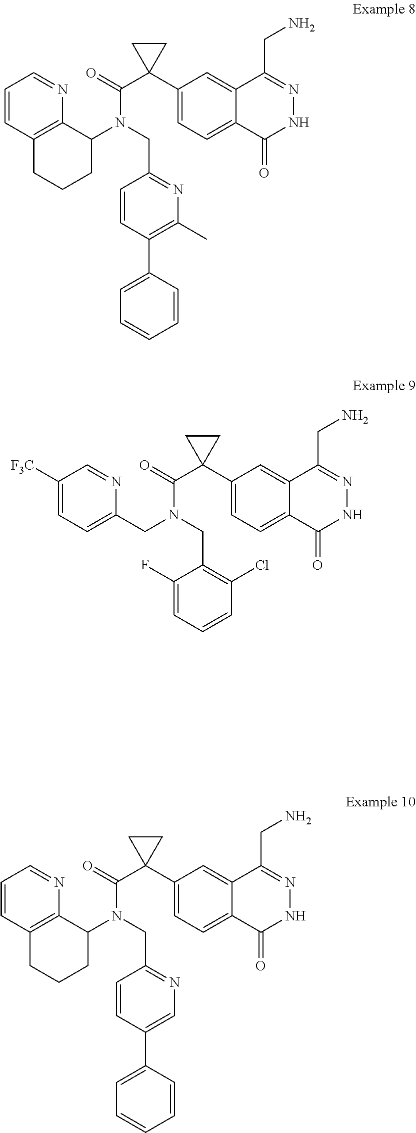 [SMILES: Cc1nc(CN(C(=O)C2(c3ccc4c(=O)[nH]nc(CN)c4c3)CC2)C2CCCc3cccnc32)ccc1-c1ccccc1 ; NCc1n[nH]c(=O)c2ccc(C3(C(=O)N(Cc4ccc(C(F)(F)F)cn4)Cc4c(F)cccc4Cl)CC3)cc12 ; NCc1n[nH]c(=O)c2ccc(C3(C(=O)N(Cc4ccc(-c5ccccc5)cn4)C4CCCc5cccnc54)CC3)cc12]

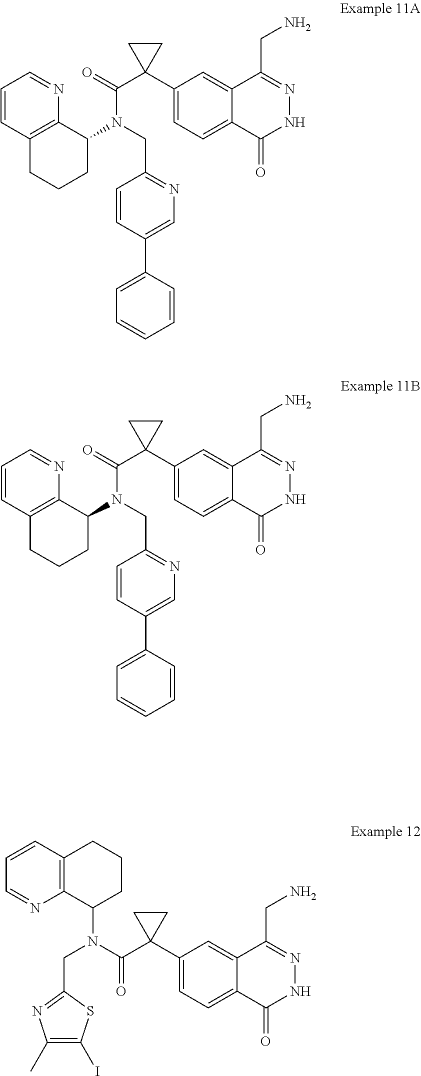 [SMILES: NCc1n[nH]c(=O)c2ccc(C3(C(=O)N(Cc4ccc(-c5ccccc5)cn4)[C@@H]4CCCc5cccnc54)CC3)cc12 ; NCc1n[nH]c(=O)c2ccc(C3(C(=O)N(Cc4ccc(-c5ccccc5)cn4)[C@H]4CCCc5cccnc54)CC3)cc12 ; Cc1nc(CN(C(=O)C2(c3ccc4c(=O)[nH]nc(CN)c4c3)CC2)C2CCCc3cccnc32)sc1I]

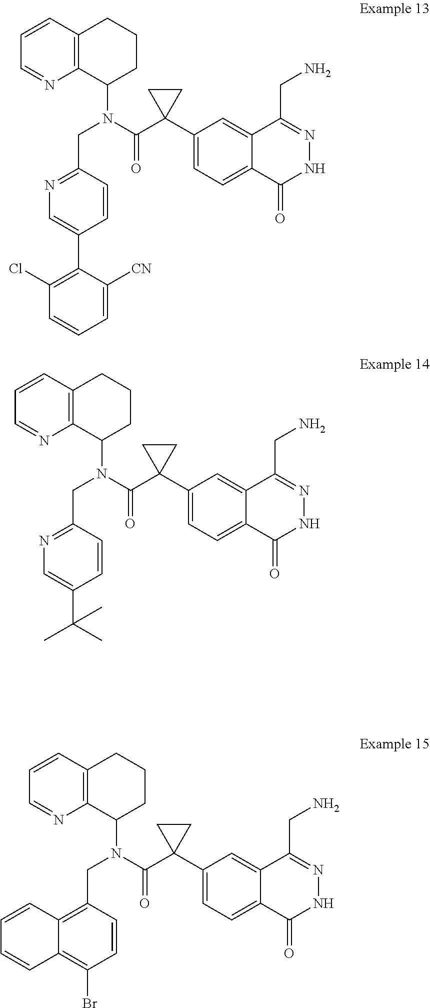

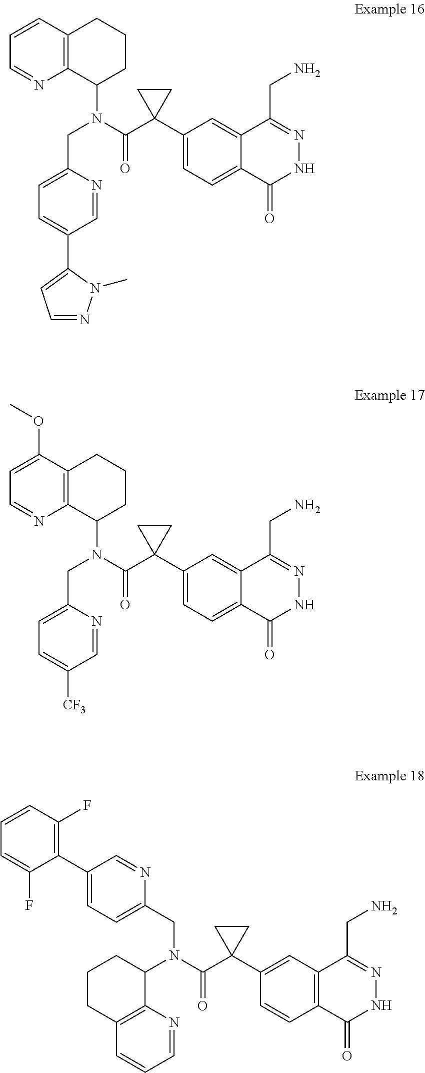 [SMILES: Cn1nccc1-c1ccc(CN(C(=O)C2(c3ccc4c(=O)[nH]nc(CN)c4c3)CC2)C2CCCc3cccnc32)nc1 ; COc1ccnc2c1CCCC2N(Cc1ccc(C(F)(F)F)cn1)C(=O)C1(c2ccc3c(=O)[nH]nc(CN)c3c2)CC1 ; NCc1n[nH]c(=O)c2ccc(C3(C(=O)N(Cc4ccc(-c5c(F)cccc5F)cn4)C4CCCc5cccnc54)CC3)cc12]

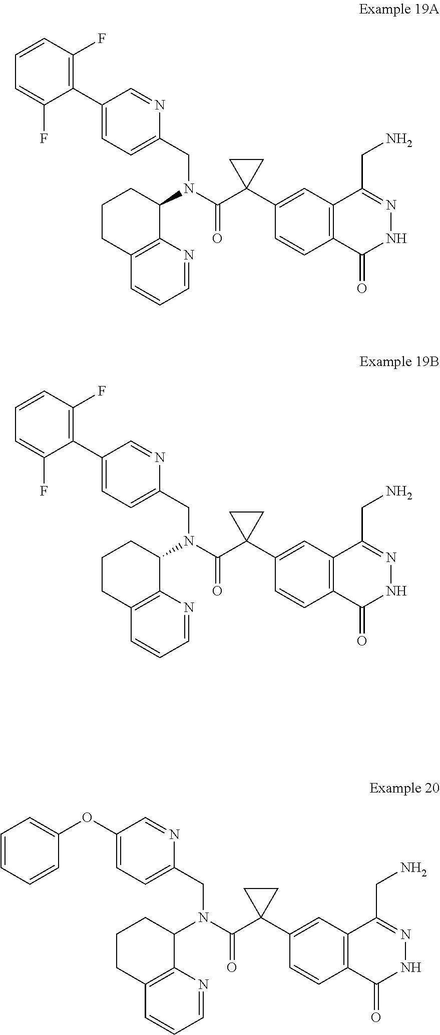 [SMILES: NCc1n[nH]c(=O)c2ccc(C3(C(=O)N(Cc4ccc(-c5c(F)cccc5F)cn4)[C@@H]4CCCc5cccnc54)CC3)cc12 ; NCc1n[nH]c(=O)c2ccc(C3(C(=O)N(Cc4ccc(-c5c(F)cccc5F)cn4)[C@H]4CCCc5cccnc54)CC3)cc12 ; NCc1n[nH]c(=O)c2ccc(C3(C(=O)N(Cc4ccc(Oc5ccccc5)cn4)C4CCCc5cccnc54)CC3)cc12]

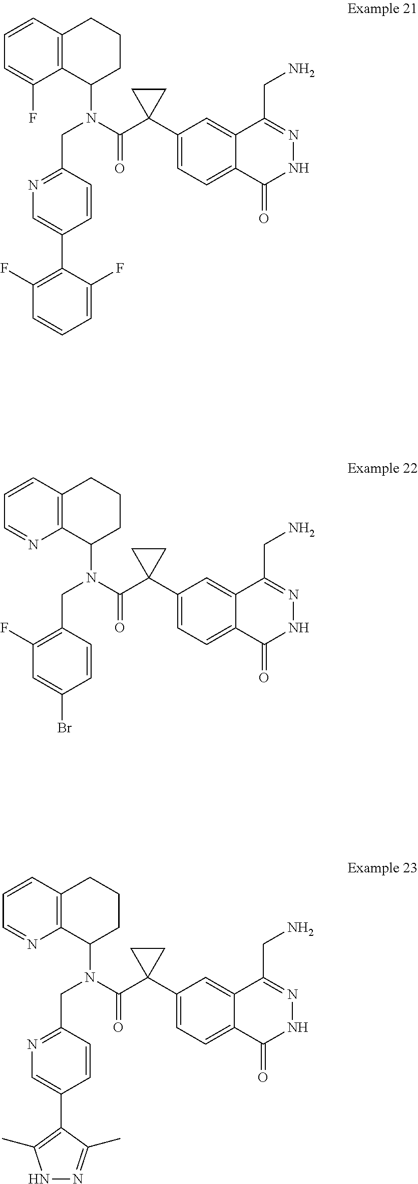 [SMILES: NCc1n[nH]c(=O)c2ccc(C3(C(=O)N(Cc4ccc(-c5c(F)cccc5F)cn4)C4CCCc5cccc(F)c54)CC3)cc12 ; NCc1n[nH]c(=O)c2ccc(C3(C(=O)N(Cc4ccc(Br)cc4F)C4CCCc5cccnc54)CC3)cc12 ; Cc1n[nH]c(C)c1-c1ccc(CN(C(=O)C2(c3ccc4c(=O)[nH]nc(CN)c4c3)CC2)C2CCCc3cccnc32)nc1]

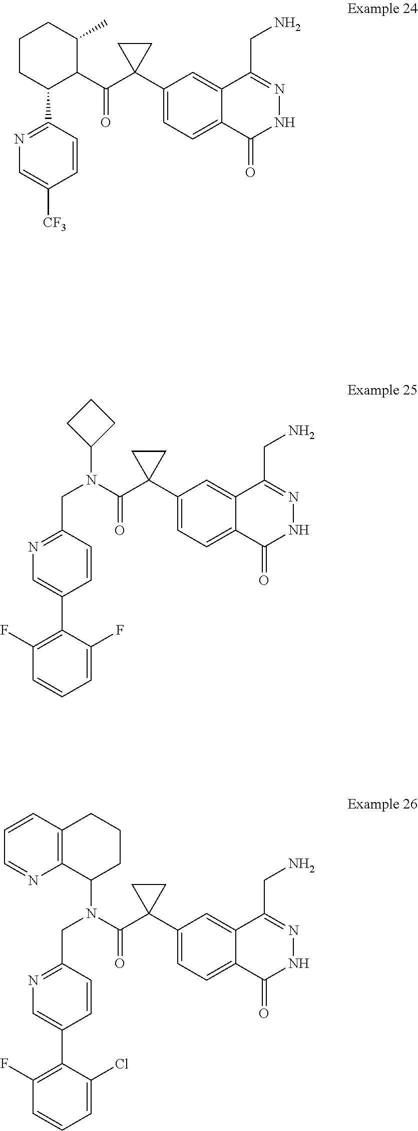 [SMILES: C[C@H]1CCC[C@@H](c2ccc(C(F)(F)F)cn2)C1C(=O)C1(c2ccc3c(=O)[nH]nc(CN)c3c2)CC1 ; NCc1n[nH]c(=O)c2ccc(C3(C(=O)N(Cc4ccc(-c5c(F)cccc5F)cn4)C4CCC4)CC3)cc12 ; NCc1n[nH]c(=O)c2ccc(C3(C(=O)N(Cc4ccc(-c5c(F)cccc5Cl)cn4)C4CCCc5cccnc54)CC3)cc12]

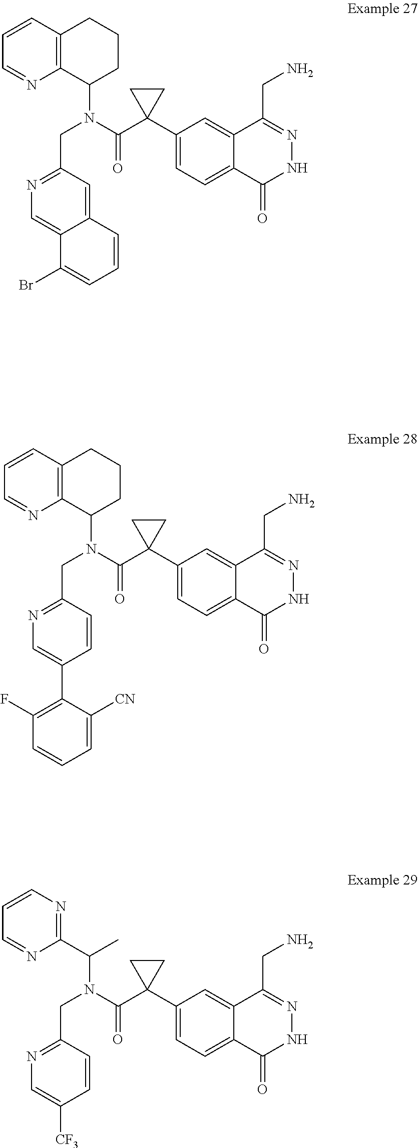 [SMILES: NCc1n[nH]c(=O)c2ccc(C3(C(=O)N(Cc4cc5cccc(Br)c5cn4)C4CCCc5cccnc54)CC3)cc12 ; N#Cc1cccc(F)c1-c1ccc(CN(C(=O)C2(c3ccc4c(=O)[nH]nc(CN)c4c3)CC2)C2CCCc3cccnc32)nc1 ; CC(c1ncccn1)N(Cc1ccc(C(F)(F)F)cn1)C(=O)C1(c2ccc3c(=O)[nH]nc(CN)c3c2)CC1]

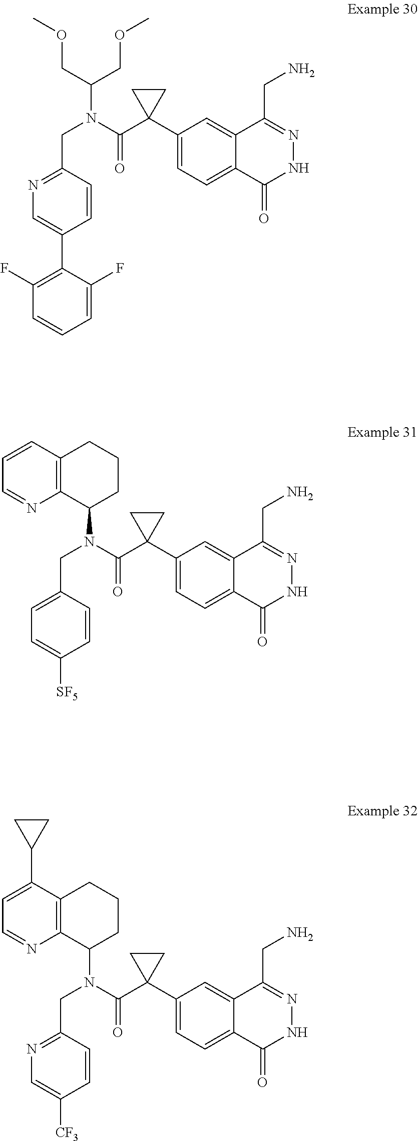

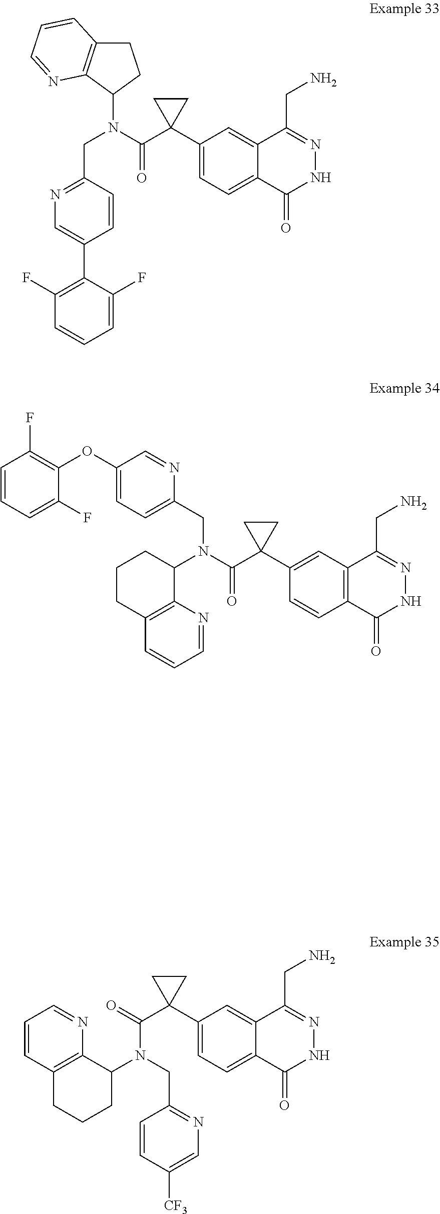 [SMILES: NCc1n[nH]c(=O)c2ccc(C3(C(=O)N(Cc4ccc(-c5c(F)cccc5F)cn4)C4CCc5cccnc54)CC3)cc12 ; NCc1n[nH]c(=O)c2ccc(C3(C(=O)N(Cc4ccc(Oc5c(F)cccc5F)cn4)C4CCCc5cccnc54)CC3)cc12 ; NCc1n[nH]c(=O)c2ccc(C3(C(=O)N(Cc4ccc(C(F)(F)F)cn4)C4CCCc5cccnc54)CC3)cc12]

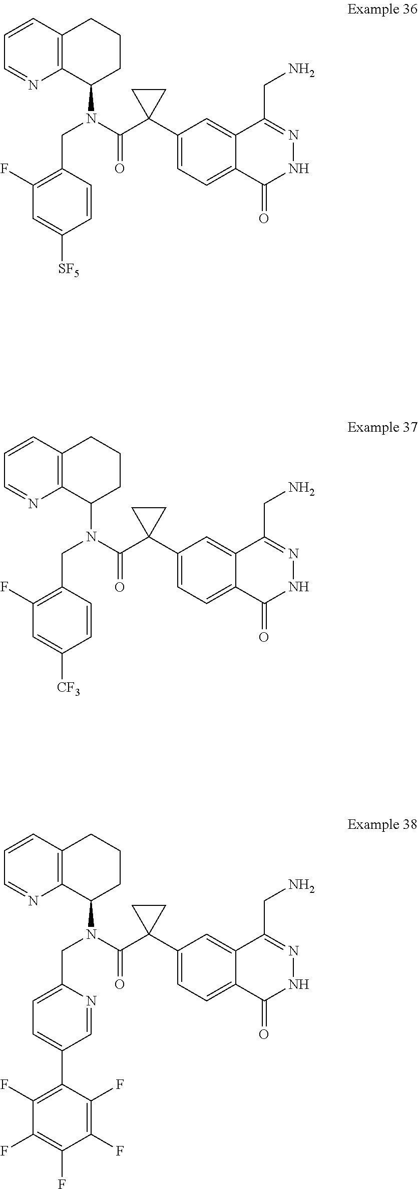

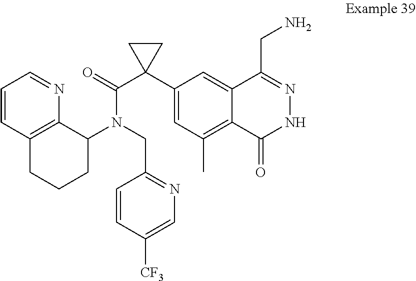 [SMILES: Cc1cc(C2(C(=O)N(Cc3ccc(C(F)(F)F)cn3)C3CCCc4cccnc43)CC2)cc2c(CN)n[nH]c(=O)c12]

15. A pharmaceutical composition comprising a compound of any one of claims 1-14 or a pharmaceutically acceptable salt, stereoisomer, tautomer or prodrug thereof, together with a pharmaceutically acceptable excipient.

16. A method of decreasing PRMT5 activity by inhibition, which comprises administering to an individual the compound according to any one of claims 1-14, or a pharmaceutically acceptable salt thereof, including the compound of formula (I) or the specific compounds exemplified herein.

17. The method of claim 16, wherein the disease is selected from cancer.

18. Use of a compound of any one of claims 1-14 or a pharmaceutically acceptable salt, stereoisomer, tautomer or prodrug thereof in the preparation of a medicament for treating a disease that is modulated by PRMT5.

19. The use of claim 18, wherein the disease is cancer.

20. The use of claim 18, wherein the disease is MTAP-null solid tumor, including but not limited to lung cancer, bladder cancer, melanoma, pancreatic cancer, esophageal cancer, gastric adenocarcinoma, breast cancer, glioblastoma, etc.
